(19) United States
(12) Patent Application Publication
(10) Pub. No.: US 2025/0255902 A1
(43) Pub. Date: Aug. 14, 2025

(54) GENETICALLY ENGINEERED B CELLS AND METHODS OF USE THEREOF

(71) Applicant: Dana-Farber Cancer Institute, Inc., Boston, MA (US)

(72) Inventors: Wayne A. Marasco, Wellesley, MA (US); Matthew Chang, Brookline, MA (US)

(21) Appl. No.: 18/845,358

(22) PCT Filed: Mar. 9, 2023
(86) PCT No.: PCT/US2023/014919

§ 371 Date: Sep. 9, 2024

Related U.S. Application Data
(60) Provisional application No. 63/318,317, filed on Mar. 9, 2022.

(51) Int. Cl. A61K 35/17; A61K 40/13; A61K 40/31; A61K 40/42; A61K 40/46; C07K 14/54; C07K 16/10; C07K 16/12; C07K 16/28; C07K 16/42; C12N 5/0781; C12N 15/86

(52) U.S. Cl. CPC A61K 35/17; A61K 40/13; A61K 40/31; A61K 40/421; A61K 40/4219; A61K 40/46; C07K 14/5434; C07K 16/1002; C07K 16/1018; C07K 16/1045; C07K 16/1289; C07K 16/2818; C07K 16/2866; C07K 16/2878; C07K 16/4258; C12N 5/0635; C12N 15/86; A61K 2239/13; A61K 2239/21; A61K 2239/22; A61K 2239/29; C07K 2317/31; C12N 2510/00; C12N 2740/15043

(57) ABSTRACT

This invention is directed to genetically engineered B cells, wherein the B cell expresses and bears on its surface a chimeric B cell receptor, and wherein the genetically engineered B cell further expresses and secretes an antibody or cytokine.

### RELATED APPLICATIONS

This application is a National Stage Entry of PCT Application No. PCT/US2023/014919, filed on Mar. 9, 2023, which claims priority from U.S. Provisional Patent Application No. 63/318,317, filed on Mar. 9, 2022, the entire contents of which is incorporated herein by reference.

### GOVERNMENT INTEREST

This invention was made with government support under W81XWH-21-1-0166 awarded by the Defense Health Agency, Medical Research and Development Branch. The government has certain rights in the invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### INCORPORATION-BY-REFERENCE OF SEQUENCE LISTING

This application contains a Sequence Listing which has been submitted electronically in XML format. The Sequence Listing XML is incorporated herein by reference. Said XML file, created on May 1, 2025, is named 5031461-000132-US2_SL.xml and is 45,813 bytes in size.

### FIELD OF THE INVENTION

This invention is directed to genetically engineered B cells, wherein the B cell expresses and bears on its surface a chimeric B cell receptor (cBCR), and wherein the genetically engineered B cell further expresses and secretes an antibody or cytokine.

### BACKGROUND OF THE INVENTION

Over the past decade, immune checkpoint blockade inhibitors (CBI) and CAR-T cells have revolutionized the way we treat cancer. While both of these therapies engage the patient's immune system, neither is able to proactively initiate an anti-tumor immune response.

### SUMMARY OF THE INVENTION

Aspects of the invention are drawn towards genetically engineered B cells.

In embodiments, the genetically engineered B cells express and bear on its surface a chimeric B cell receptor (cBCR), and wherein the genetically engineered B cell further expresses and secretes an antibody or cytokine.

In embodiments, the chimeric B cell receptor comprises an extracellular domain, a transmembrane domain, and an intracellular signaling domain.

In embodiments, the extracellular domain is an antibody or antibody fragment. In embodiments, the antibody is a nanobody, bi-specific, scFv or Fab. In embodiments, the antibody is specific for a tumor associated antigen. For example, the tumor associated antigen is selected from the group consisting of CAIX, BCMA, CD138, PD-L1, PD-L2, VEGF, CD70, CD99, CEA, Her-2, GD2, CD171, αFR, PMSA, IL13α, MSLN, TAG-72, and TROP2. For example, the bi-specific antibody is specific for PD-1 and CTLA4, PD-1 and TIGIT, TIGIT and CCR4, GITR and TIGIT, or PD-1 and CCR4. In embodiments, the antibody is an anti-IGHV 1-69 antibody. In embodiments, the antibody is specific for an infectious disease associated antigen, such as HA1, HA2, NA, or spike protein. For example, the infectious disease is a viral disease, such as influenza, coronavirus, HIV, or tuberculosis.

In embodiments, the expression of the antibody or cytokine that is secreted is controlled by an inducible response element. For example, the inducible response element is an NFAT or NFκB response element. In embodiments, the antibody is a checkpoint blockade modulator. In embodiments, the antibody is a checkpoint blockade inhibitor. In embodiments, the antibody is specific for CA-9, PD-1, PD-L1, PD-L2, CTLA4, TIGIT, VISTA, CD70, TIM-3, LAG-3, CD40L, CCR4, GITR, or CXCR4. In embodiments, the antibody is specific for HA1, HA2, NA, or spike protein. In embodiments, the cytokine is selected from the group consisting of IL-2, IL-7, IL-12, IL-15, IL-18, CD40-L, or BAFF. In embodiments, the antibody comprises a monoclonal antibody. In embodiments, the antibody comprises a humanized antibody. In embodiments, the antibody comprises a nanobody, scFv, Fab, an antibody-cytokine fusion, or a bi-specific antibody. For example, the bi-specific antibody is specific for PD-1 and CTLA4, PD-1 and TIGIT, TIGIT and CCR4, GITR and TIGIT, or PD-1 and CCR4. For example, the antibody-cytokine fusion protein comprises anti-PD1-scIL12.

Aspects of the invention are also drawn towards a nucleic acid encoding a first polypeptide and a second polypeptide, wherein the first polypeptide comprises a chimeric B cell receptor, wherein the chimeric B cell receptor comprises an extracellular domain, a transmembrane domain, and an intracellular signaling domain, and wherein the second polypeptide comprises an antibody or cytokine.

In embodiments, the extracellular domain is an antibody or antibody fragment. In embodiments, the antibody is a nanobody, bi-specific, scFv or Fab. In embodiments, the antibody is specific for a tumor associated antigen. For example, the tumor associated antigen is selected from the group consisting of CAIX, BCMA, CD138, PD-L1, PD-L2, VEGF, CD70, CD99, CEA, Her-2, GD2, CD171, αFR, PMSA, IL13α, MSLN, TAG-72, and TROP2. For example, the bi-specific antibody is specific for PD-1 and CTLA4, PD-1 and TIGIT, TIGIT and CCR4, GITR and TIGIT, or PD-1 and CCR4. In embodiments, the antibody is an anti-IGHV 1-69 antibody. In embodiments, the antibody is specific for an infectious disease associated antigen, such as HA1, HA2, NA, or spike protein. For example, the infectious disease is a viral disease, such as influenza, coronavirus, HIV, or tuberculosis.

In embodiments, the expression of the antibody or cytokine that is secreted is controlled by an inducible response element. For example, the inducible response element is an NFAT or NFκB response element. In embodiments, the antibody is a checkpoint blockade modulator. In embodiments, the antibody is specific for CA-9, PD-1, PD-L1, PD-L2, CTLA4, TIGIT, VISTA, CD70, TIM-3, LAG-3, CD40L, CCR4, GITR, or CXCR4. In embodiments, the antibody is specific for HA1, HA2, NA, or spike protein. In embodiments, the cytokine is selected from the group consisting of IL-2, IL-7, IL-12, IL-15, IL-18, CD40-L, or BAFF. In embodiments, the antibody comprises a monoclonal antibody. In embodiments, the antibody comprises a humanized antibody. In embodiments, the antibody comprises a nanobody, scFv, Fab, or a bi-specific antibody. For example, the bi-specific antibody is specific for PD-1 and CTLA4, PD-1 and TIGIT, TIGIT and CCR4, GITR and TIGIT, or PD-1 and CCR4.

Aspects of the invention are further drawn towards a vector comprising the nucleic acid as described herein. In embodiments, the vector is a lentiviral vector or an adeno-associated virus (AAV) vector. Lentivirus and AAV are viruses that can be useful as vectors for gene therapy, such as with the CRISPR/Cas system.

Still further, aspects of the invention are drawn towards a cell comprising the vector as described herein.

Also, aspects of the invention are drawn towards a composition comprising a first expression vector and a second expression vector, wherein the genome of the first expression vector comprises a nucleotide sequence encoding a chimeric B cell receptor, and wherein the genome of the second expression vector comprises a nucleotide sequence encoding an antibody or cytokine. In embodiments, the first expression vector and the second expression vector are lentiviral vectors or adeno-associated viral vectors.

In embodiments, the nucleotide sequence encoding an inducible response element is operatively linked to the nucleotide sequence encoding an antibody or cytokine. For example, the inducible response element is the NFAT or NFκB response element.

In embodiments, the chimeric B cell receptor comprises an extracellular domain, a transmembrane domain, and an intracellular signaling domain. In embodiments, the extracellular domain is an antibody or antibody fragment. In embodiments, the antibody is a nanobody, scFv, or Fab. In embodiments, the antibody is specific for a tumor associated antigen. For example, the tumor associated antigen is selected from the group consisting of CAIX, BCMA, CD138, PD-L1, PD-L2, VEGF, CD70, CD99, CEA, Her-2, GD2, CD171, αFR, PMSA, IL13α, MSLN, TAG-72, and TROP2. In embodiments, the antibody is an anti-IGHV 1-69 antibody. In embodiments, the antibody is specific for an infectious disease associated antigen, such as HA1, HA2, NA, or spike protein. For example, the infectious disease is a viral disease, such as influenza, coronavirus, HIV, or tuberculosis.

In embodiments, the expression of the antibody or cytokine that is secreted is controlled by an inducible response element. For example, the inducible response element is an NFAT or NFκB response element. In embodiments, the antibody is a checkpoint blockade modulator. In embodiments, the antibody is a checkpoint blockade inhibitor. In embodiments, the antibody is specific for CA-9, PD-1, PD-L1, PD-L2, CTLA4, TIGIT, VISTA, CD70, TIM-3, LAG-3, CD40L, CCR4, GITR, or CXCR4. In embodiments, the antibody is specific for HA1, HA2, NA, or spike protein. In embodiments, the cytokine is selected from the group consisting of IL-2, IL-7, IL-12, IL-15, IL-18, CD40-L, or BAFF. In embodiments, the antibody comprises a monoclonal antibody. In embodiments, the antibody comprises a humanized antibody. In embodiments, the antibody comprises a nanobody, scFv, Fab, an antibody-cytokine fusion, or a bi-specific antibody. For example, the bi-specific antibody is specific for PD-1 and CTLA4, PD-1 and TIGIT, TIGIT and CCR4, GITR and TIGIT, or PD-1 and CCR4. For example, the antibody-cytokine fusion comprises anti-PD1-scIL 12.

Aspects of the invention are also drawn to a method of making a population of genetically engineered B cells. In embodiments, the method comprises: isolating a population of B cells from a subject, and transducing the population of B cells with the vector(s) as described herein, thereby producing a population of genetically engineered B cells. Embodiments can further comprise the step of activating the population of B cells prior to transduction. Embodiments can further comprise the step of culturing the population of genetically engineered B cells. Embodiments can further comprise the step of administering the population of genetically engineered B cells to a subject in need thereof.

Further, aspects of the invention are drawn to methods of treating a subject afflicted with cancer by administering to a subject the genetically engineered B cell as described herein, the nucleic acid as described herein, or the composition as described herein.

Still further, aspects of the invention are drawn to methods of preventing cancer in a subject by administering to a subject the genetically engineered B cell as described herein, the nucleic acid as described herein, or the composition as described herein. For example, the cancer is BCLL, NSCLC, ccRCC, mesothelioma.

Aspects of the invention are drawn to methods of treating a subject afflicted with an infectious disease by administering to a subject the genetically engineered B cell as described herein, the nucleic acid as described herein, or the composition as described herein.

Still further, aspects of the invention are drawn to methods of preventing an infectious disease by administering to a subject the genetically engineered B cell as described herein, the nucleic acid as described herein, or the composition as described herein. For example, the infectious disease is a viral disease, such as influenza, coronavirus, HIV, or tuberculosis.

Other objects and advantages of this invention will become readily apparent from the ensuing description.

### BRIEF DESCRIPTION OF THE FIGURES

FIG. 1 shows a schematic of CASS B cell therapy for non-small cell lung cancer. In this example, anti-MSLN is the targeting moiety, and anti-TIGIT/anti-PD1 bi-specific antibody is the secreted moiety.

FIG. 2 shows characterization of exemplary antibodies for CASS B cell development. Panel A) Anti-PD1 mAb is comparable with pembrolizumab in MLR assay. Panel B) BLI assay demonstrates that antibodies block the TIGIT/CD155 binding interaction (purple) and share an epitope with the control Abs (blue). Panel C) MSLN+ cell binding curve (left) and BLI based kinetic measurements (right) demonstrate that one clone (Gly1-2-H4) binds a conformational epitope only present on the GPI linked form of MSLN.

FIG. 3 shows design of a bi-specific antibody (bsAb). Panel A) Schematic of bsAb with the different scFvs colored red or blue. Diagram of the experimental layout (panel B) and resultant binding cure (panel C) of a dual binding assay demonstrating functionality of both sides of the bsAb.

FIG. 4 shows CASS B cell construct testing and B cell transduction. Panel A) Cells expressing the engineered IgG-BCR construct can bind soluble HA. Panel B) Lentivirus can be used to achieve high transduction efficiency using multiple DNA constructs and donors. Panel C) A reporter system has been engineered in Jurkat cells using an NFAT/NFkB inducible response element that shows increasing levels of GFP expression with various stimulatory conditions. FIG. 4 discloses “6×His” as SEQ ID NO: 22.

FIG. 5 shows summary of data on hG6.3 targeting moiety, biological studies and CASS B cell design and strategy: (Panel A) The hG6.3 core binding idiotope to D80, an IGHV1-69 mAb, which comprises of CDR-H2 residues M53 to N58 (green), bound to hG6.3 (blue) in the crystal structure. (Panel B) Preferential recognition of unmutated IGHV1-69 B-CLL patient sample by hG6.3-CAR T cells (G36 is anti-CAIX control): (Panel C) Schematic of CASS vector and different CBI payloads and bi-specific Ab derivatives, (Panel D) Schematic of CASS B cell therapy. B cells are recovered by leukapheresis, transduced to express chimeric BCR targeting cancer cells and encoded to secrete the optimized CBI payload that in determine by ex vivo PDOTS and in vivo humanized mouse studies.

FIG. 6 shows membrane expression (panel A) and HA binding (panel B) of 293T cells.

FIG. 7 provides exemplary combined constructs of embodiments of the invention. FIG. 7 discloses SEQ ID NOS 23-25, 14, 26, 23-24, 27, 14, 26, 23-24, 28, 14 and 26, respectively, in order of appearance.

FIG. 8 provides exemplary split vector constructs of embodiments of the invention. FIG. 8 discloses SEQ ID NOS 29, 14, 26, 30, 14, 26, 31, 14, 26, 23-24 and 32, respectively, in order of appearance.

FIG. 9 shows schematics of current immunotherapies. Panel A is adapted from Shifaa M. Abdin et al. J Immunother Cancer 2021:9:e002741. Panel B is adapted from Larson, R. C., Maus, M. V. Nat Rev Cancer 21, 2021. Panel C is adapted from Zhang, C., Hu, Y., Xiao, W. et al. Cell Mol Immunol 2021.

FIG. 10 shows an illustration of the function of B cells in the immune system adapted from Li Rui et al, Frontiers in Immunology, 2016.

FIG. 11 shows an illustration of a B cell signaling pathway adapted from Balaji, S., Ahmed, M., Lorence, E. et al. J Hematol Oncol, 2018.

FIG. 12 shows an illustration of Chimeric Antibody Signaling and Secreting (CASS) B cells.

FIG. 13 shows an illustration of a first run of B cell transduction adapted from Howell Moffett et al, Science Immunology, 2019. Media: StemMACS HSC Expansion Media XF+5% FBS+IL4+CD40L-hex+ODN2006.

FIG. 14 shows an illustration of a design of Chimeric Antibody Signaling and Secreting (CASS) B cells.

FIG. 15 shows an illustration of constructs. FIG. 15 discloses SEQ ID NOS 33-38 and 37-40, respectively, in order of appearance.

FIG. 16 shows representative data of a first test of B cell transduction. Constructs used in this experiment contained an internal BGH domain, which results in premature termination of the construct and failure to incorporate via the LTRs. As such, high gene expression is seen immediately after transduction, however within 2 weeks expression has decreased to near 0. This indicates a lack of genetic integration, rather expression via transient “transfection” with the lentivirus.

FIG. 17 shows schematics of redesigning the CASS B construct. Following the results in FIG. 16, the BGH poly A was removed to allow for stable genomic integration following transduction.

FIG. 18 shows representative data of transduction check after removing BGH. Isolate and activate B cells: Nov. 11, 2021; Transduce: Nov. 12, 2021; and Stain: Nov. 19, 2021. The virus was pseudotyped with VSVG and the BGH was removed. B cells were transduced 18-24 hours post activation. However, they are negative 7 days post transduction.

FIG. 19 shows representative graphs of data showing the duration and manner B cell stimulation plays a role in transduction. Adapted from Bovia, Fabrice et al, Gene Therapy 2013.

FIG. 20 shows representative data of optimal envelope protein for B cell transduction. VSVG: low density lipoprotein receptor (LDL-R); GALV: Sodium-dependent phosphate transporter 1 and 2 (GLVR-1/2); and BaEV: neutral amino acid (aa) transporter 1 and 2 (ASCT-1/2). Panel A is adapted from Caeser, R., Di Re, M., Krupka, J. A. et al. Nat Commun 2019. Panel B is adapted from Levy C et al, J Thromb Haemost 2016.

FIG. 21 shows a schematic and representative data of a transfer plasmid optimized for B cell transduction.

FIG. 22 shows a schematic of two transduction protocols. The Bovia protocol utilizes EL4-B5 feeder cells while the Moffett protocol utilizes soluble CD40L. Moffett: 1. H. F. Moffett, C. K. Harms, K. S. Fitzpatrick, M. R. Tooley, J. Boonyaratanakornkit, J. J. Taylor, B cells engineered to express pathogen-specific antibodies protect against infection. Sci. Immunol. 4 (2019), doi:10.1126/sciimmunol.aax0644, 31101673. Bovia: 1. F. Bovia, P. Salmon, T. Matthes, K. Kvell, T. H. Nguyen, C. Werner-Favre, M. Barnet, M. Nagy, F. Leuba, J. F. Arrighi, V. Piguet, D. Trono, R. H. Zubler, Efficient transduction of primary human B lymphocytes and nondividing myeloma B cells with HIV-1-derived lentiviral vectors. Blood. 101, 1727-1733 (2003), 12406892.

FIG. 23 shows representative data of a transduction check 9 days post. CD19 was used to separate B cells from EL4-B5 and virus was pseudotyped using 3 different envelope proteins (VSVG, BaEV, GALV). For cells activated via the Bovia protocol, it can be seen that high transduction titers are seen in the EL4-B5 feeder cells (CD19-) but not in human B cells (CD19+). Following the protocol adopted from Moffett, a high level of GFP expression seen in CD19+ B cells indiciating efficient transduction using BaEV pseudotyped virus but not GALV or VSVG. RetroNectin reagent is a recombinant human fibronectin fragment sold by TakaraBio. Without wishing to be bound by theory, it enhances viral transduction through binding of viral particles to a heparin-binding domain and binding to cellular integrins VLA-4 and/or VLA-5 via the C-domain (https://www.takarabio.com/learning-centers/gene-function/t-cell-transduction-and-culture/retronectin-faqs). However in this assay we did not see a benefit of using RetroNectin for transduction.

FIG. 24 shows representative data of a day 4 transduction check. Cells were transduced with BaEV pseudotyped lentivirus. The first column uses a LeGO GFP vector which constitutively expresses GFP. The pHAGE-F10-NFkB is a CASS B cell vector expressing GFP under control of an NFkB/NFAT response element. pHAGE-F10 is only the extracellular binding domain of the CASS B cell. Importantly the CASS B cell vector shows both binding of QBend10 (epitope marker via RQR8) and expression of GFP.

FIG. 25 shows representative data of HA Binding to F10-CASS B cell. 250 k cells were washed with PBS and resuspended in 5 uM ZombieViolet dye (in PBS) for 20 min at RT. Cells were quenched with media containing FBS, and then resuspended in 100 ul with FcX and 4 μg/ml biotinylated H1 stem. After incubation at 4 C for 1 hour, cells were washed and stained with QBend10-FITC and APC-streptavidin. Importantly, nearly all QBend10+ cells also bind HA, indicating that transduced cells express both functional chimeric BCRs and the epitope tag which are separated by T2A.

FIG. 26 shows representative data of inducible expression of GFP. Transduced B cells were activated for 4 days and then inducible GFP expression was measured 8 days post transduction. As seen here, GFP expression of the NFAT-NFkB construct does not change with the addition BCR stimulation (anti-IgM or HA+Strep) compared to media only. The 3×3 NFAT construct does not show any change in GFP expression under any condition.

FIG. 27 shows schematics of representative payload constructs built. ZsG is used for platform optimization, anti-PD(L)1 would be anti-cancer therapy, and aSARS would be either an irrelevant control in cancer therapy or anti-SARS therapy for infectious disease therapies.

FIG. 28 shows representative data of CASS B cells secretion of scFv-Fcs. B cells seeded at 1E6 cells/ml in stim media on May 24, 2022 and harvested on May 28, 2022. High levels of IgG expression were observed in all wells, however antigen specific scFv-Fcs were detected for anti-PD1 P4B3m3. Based on the ELISA results with purified proteins, this is likely due to the low affinity of mAb 42 (aPDL1) and the wrong spike target selected for 11A. 11A targets GD03 SARS but a different strain was used for the ELISA.

### DETAILED DESCRIPTION OF THE INVENTION

Described herein are compositions and methods relating to Chimeric Antibody Signaling and Secreting (CASS) B cells as a targeted and inducible platform to secrete immunomodulatory polypeptides at a target site. For example, CASS B cells can express an engineered, tumor targeting B cell receptor on its surface and, upon engagement, can secrete high levels of a dual-targeted bi-specific checkpoint blockade modulator antibody locally at the tumor site. As B cells also serve as professional antigen presenting cells, the CASS B cells can also process and present antigens on MHC class II molecules, further enhancing immune cell recognition of the tumor and assisting in neoantigen spreading. A key component of immunologic memory, CASS B cells can simultaneously recruit a wide range of immune cells and reverse tumor infiltrating lymphocyte exhaustion, providing a robust and lifelong surveillance program protecting against tumor metastasis and recurrence. Embodiments of the CASS B cell platform can be used to prevent and treat cancer and infectious diseases.

Detailed descriptions of one or more embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein, the term “about” can refer to approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

### Chimeric B-Cell Receptor

Aspects of the invention are drawn to genetically engineered B cells that are modified to express and bear on its surface a chimeric B cell receptor. In embodiments, the genetically modified B cell can comprise a single chimeric B cell receptor targeting one antigen, or a single chimeric B cell receptor targeting two or more antigens (e.g., a bi-specific chimeric B cell receptor, or a multispecific chimeric B cell receptor). In some embodiments, the cells comprise a split chimeric B cell receptor, such as two different scFvs expressed on the B cell surface with different costimulation domains. Further, some embodiments comprise a fine-tuned chimeric B cell receptor.

A split chimeric B cell receptors can comprise two or more chimeric B cell receptors on the surface of a cell, such as a B cell. The chimeric B cell receptors can be specific for two or more antigens. In this example, the first chimeric B cell receptors is specific for a first antigen, and the second chimeric B cell receptors is specific for a second antigen. As described herein, the chimeric B cell receptors can be in any orientation desired. For example, the first chimeric B cell receptors can be specific for the second antigen and the second chimeric B cell receptors can be specific for the first antigen. The first and the second chimeric B cell receptors can be expressed from a single nucleic acid construct. In such an example, a nucleic acid encoding a cleavable linker can be located between the nucleic acids encoding the first and the second chimeric B cell receptors. In other embodiments, the two chimeric B cell receptors can be expressed in the same cell but from two separate nucleic acid constructs.

In embodiments, the chimeric B cell receptor comprises an extracellular domain, a transmembrane domain, and an intracellular domain.

A modified B cell receptor called chimeric B cell receptor, such as a B cell receptor containing an antibody or antibody fragment previously selected by high affinity against a specific disease associated antigen, is a powerful new approach against diseases. As B cells serve as professional antigen presenting cells, they can process and present antigens on MHC class II molecules, enhancing immune cell recognition of the tumor and assisting in neoantigen spreading. A key component of immunologic memory, CASS B cells will simultaneously recruit a wide range of immune cells and reverse tumor infiltrating lymphocyte exhaustion, providing a robust and lifelong surveillance program protecting against tumor metastasis and recurrence. In particular cases, the B cell can include a receptor that is chimeric, non-natural and engineered at least in part by the hand of man. In particular cases, the engineered chimeric B cell receptor has one, two, three, four, or more components, and in some embodiments the one or more components facilitate targeting or binding of the B cell to one or more antigen-comprising cells.

The chimeric B cell receptor according to the disclosure comprises at least one transmembrane polypeptide comprising at least one extracellular ligand-binding domain and; one transmembrane polypeptide comprising at least one intracellular signaling domain; such that the polypeptides assemble together to form a chimeric B cell receptor.

The term “extracellular ligand-binding domain” or “extracellular domain” as used herein can refer to an oligo- or polypeptide that can bind a ligand. The domain can interact with a cell surface molecule. For example, the extracellular ligand-binding domain can be chosen to recognize a ligand that acts as a cell surface marker on target cells associated with a particular disease state. For example, the disease state can be cancer, and the target ligand can be a cancer associated antigen. In another example, the disease state can be an infectious disease, and the target ligand can be an infectious disease associated antigen. In embodiments, the extracellular ligand-binding domain can comprise an antigen binding domain or antigen recognition domain derived from an antibody against an antigen of the target. The antigen binding domain or antigen recognition domain can be an antibody fragment. An “antibody fragment” can be a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies: linear antibodies: single-chain antibody molecules (e.g. scFv); and multispecific antibodies formed from antibody fragments. For example, embodiments can comprise a chimeric B cell receptor with two scFvs as the antigen recognition domains. Embodiments can also comprise a chimeric B cell receptor with one scFv as the antigen recognition domain. Further, embodiments can comprise a chimeric B cell receptor with a bi-specific antibody as the antigen recognition domain. For example, the bi-specific antibody can be specific for PD-1 and CTLA4, PD-1 and TIGIT, TIGIT and CCR4, GITR and TIGIT, or PD-1 and CCR4. For example, the bi-specific antibody can be specific for HA and NA, or influenza HA and coronavirus spike(S) or SARS2. Bi-specific or cross-reactive antibodies are known in the art. See, for example, Pilewski, Kelsey A., et al. “Functional HIV-1/HCV cross-reactive antibodies isolated from a chronically co-infected donor.” Cell Reports 42.2 (2023).

The antigen recognition domain can be directed towards any antigen target of interest. In embodiments, the antigen target of interest is on the surface of a cell, such as the surface of a cancer cell (i.e., tumor associated antigen). The antigen target of interest can also be associated with an infection disease (i.e., infectious disease associated antigen). Non-limiting examples of antigen targets comprise TIGIT, PD-1, CAIX, BCMA, CD138, PD-L1, PD-L2, VEGF, CD70, CD99, CEA, Her-2, GD2, CD171, αFR, PMSA, IL13α, MSLN, TAG-72, TROP2, B7H3, B7H4, CD27, CD28, CD40, CD40L, CD47, CD122, CCR4, CTLA-4, GITR, GITRL, ICOS, ICOSL, LAG-3, LIGHT, OX-40, OX40L, TIM3, 4-1BB, VISTA, HEVM, BTLA, and KIR. In embodiments, the antigen target comprises CAIX. In embodiments, the antibody targets mesothelin.

Exemplary antibody compositions (e.g., VH and/or VL sequences or fragments thereof) that are useful for the design of chimeric B cell receptor as described herein include, but are not limited to:
anti-CAIX antibodies described in PCT/US2006/046350 and PCT/US2015/067178
anti-CXCR4 antibodies described in PCT/US20006/005691
anti-CCR4 antibodies described in PCT/US2008/088435, PCT/US2013/039744, and PCT/US2015/054202
anti-PD-L1 antibodies described in PCT/US2008/088435 and PCT/US2020/062815
anti-PD-1 antibodies described in PCT/US2020/037791 and PCT/US2020/037781
anti-GITR antibodies described in PCT/US2017/043504
anti-claudin-4 antibodies described in PCT/US2019/022272
anti-MUC1 antibodies described in PCT/US2020/037783
anti-TIGIT antibodies described in U.S. provisional patent application 63/242,992
anti-IGHV 1-69 antibodies described in PCT/US2011/038970
anti-influenza antibodies described in PCT/US2008/085876 and PCT/US2016/026800
anti-SARS-COV-2
(each of the applications which are incorporated herein by reference in their entireties).

The term “antibody” herein is used in the broadest sense and can refer to immunoglobulin molecules and immunologically active portions of immunoglobulin (Ig) molecules, i.e., molecules that contain an antigen binding site that specifically binds (immunoreacts with) an antigen. “Specifically binds” or “immunoreacts with” can refer to the antibody reacting with one or more antigenic determinants of the desired antigen and does not react with other polypeptides. Antibodies of the disclosure can include, but are not limited to, polyclonal, monoclonal, humanized, fully human, mosaic, bi-specific, multispecific, chimeric, dAb (domain antibody), single chain antibodies, Fab, Fab′ and F(ab′)2 fragments, scFvs, diabodies, minibodies, scFv-Fc fusions, and Fab expression libraries. Unless specified to the contrary, any reference to “antibody” or “antibodies” made herein encompasses, for example, any (or all) of these molecules so long as they exhibit the desired antigen-binding activity.

Embodiments as described herein can comprise multispecific antibodies. The term “multispecific antibody” can refer to an antibody that can bind specifically to different types of epitopes. More specifically, multispecific antibodies are antibodies having specificity to at least two different types of epitopes, and, in addition to antibodies recognizing different antigens, antibodies recognizing different epitopes on the same antigen are also included. (For example, when the antigens are heterologous receptors, multispecific antibodies bind to different domains constituting the heterologous receptors: alternatively, when the antigens are monomers, multispecific antibodies bind to multiple sites on the monomer antigens.) For example, a multispecific antibody can be a pentameric IgM antibody in which each dimer represents an antibody against a different epitope or target protein.

In embodiments, the antibody comprises a modular tetrameric/tetravalent bi-specific antibody as described in WO 2018/071913, which is incorporated by reference herein in its entirety. For example, the tetravalent bi-specific antibody is a dimer of a bi-specific scFv fragment including a first binding site for a first antigen, and a second binding site for a second antigen. For example, the bi-specific antibody can be specific for PD-1 and CTLA4, PD-1 and TIGIT, TIGIT and CCR4, or PD-1 and CCR4. The two binding sites can be joined together via a linker domain. In embodiments, the scFv fragment is a tandem scFv, the linker domain includes an immunoglobulin hinge region (e.g., an IgG₁, an IgG₂, an IgG₃, and an IgG₄ hinge region) amino acid sequence. In embodiments, the immunoglobulin hinge region amino acid sequence can be flanked by a flexible linker amino acid sequence, e.g., having the amino acid sequence (GGGS)ₓ₁₋₆ (SEQ ID NO: 1), (GGGGS)ₓ₁₋₆ (SEQ ID NO: 2), and GSAGSAAGSGEF (SEQ ID NO: 3). In embodiments, the linker domain includes at least a portion of an immunoglobulin Fc domain, e.g., an IgG₁, an IgG₂, an IgG₃, and an IgG₄ Fc domain. The at least a portion of the immunoglobulin Fc domain can be a CH2 domain. The Fc domain can be linked to the C-terminus of an immunoglobulin hinge region (e.g., an IgG₁, an IgG₂, an IgG₃, and an IgG₄ hinge region) amino acid sequence. The linker domain can include a flexible linker amino acid sequence (e.g., (GGGS)ₓ₁₋₆ (SEQ ID NO: 1), (GGGGS)ₓ₁₋₆ (SEQ ID NO: 2), and GSAGSAAGSGEF (SEQ ID NO: 3)) at one terminus or at both termini.

In embodiments, the antibody comprises a mosaic antibody. A mosaic antibody is one in which the external amino acid residues of an antibody of one species are rationally replaced or “mosaicked” by the external amino acid residues of an antibody of a second species such that the antibody of the first species is not immunogenic in the second species, thereby reducing the immunogenicity of the antibody. Since the antigenicity of a protein depends primarily on its surface properties, the immunogenicity of an antibody can be reduced by substituting exposed residues that differ from those typically found in antibodies of another mammalian species. Reasonable substitution of this external residue should have little or no effect on the internal domain or on inter-domain contacts. Thus, since the changes are limited to variable region framework residues, ligand binding properties should not be affected. This process is called “mosaicism” since only the outer surface or skin of the antibody is altered and the supporting residues remain undisturbed.

A single chain Fv (“scFv”) polypeptide molecule is a covalently linked VH::VL heterodimer, which can be expressed from a gene fusion including VH- and VL-encoding genes linked by a peptide-encoding linker. (See Huston et al. (1988) Proc Nat Acad Sci USA 85(16):5879-5883). A number of methods have been described to discern chemical structures for converting the naturally aggregated, but chemically separated, light and heavy polypeptide chains from an antibody V region into an scFv molecule, which will fold into a three-dimensional structure substantially similar to the structure of an antigen-binding site. See, e.g., U.S. Pat. Nos. 5,091,513:5,132,405; and 4,946,778.

Bi-specific antibodies can be prepared as full-length antibodies or antibody fragments (e.g. F(ab′)2 bi-specific antibodies). Methods for making bi-specific antibodies are known in the art. See for example U.S. Pat. No. 8,329,178, which is incorporated herein by reference in its entirety.

Antibody molecules obtained from humans relate to any of the classes IgG, IgM, IgA, IgE and IgD, which differ from one another by the nature of the heavy chain present in the molecule. Certain classes have subclasses as well, such as IgG1, IgG2, and others. Furthermore, in humans, the light chain may be a kappa chain or a lambda chain.

The term “antigen-binding site,” or “binding portion” can refer to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” can refer to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

In embodiments, said extracellular ligand-binding domain is a single chain antibody fragment (scFv) comprising the light (VL) and the heavy (VH) variable fragment of a target antigen specific monoclonal antibody joined by a flexible linker. The skilled artisan will recognize that embodiments can comprise different linkers which are typically known in the art. See, for example, Chen. et al. “Fusion protein linkers: property, design and functionality.” Advanced drug delivery reviews 65.10 (2013): 1357-1369, which is incorporated by reference herein in its entirety. For example, using different linkers will allow one to fine tune the dual-targeting chimeric B cell receptor construct. The linker length can vary depending on the antibodies of the dual-targeting chimeric B cell receptor construct, their angle of approach to the target epitope, topography of the target on the tumor cell membrane. For example, the flexible linkers can include GGGS1 (SEQ ID NO: 4), GGGGS3 (SEQ ID NO: 5), GGGGS5 (SEQ ID NO: 6), or IgG1 hinge. In some embodiments, the number of Gs in the linker can be 2, 3, 4, 5, 6, or 7 in combination with either S1, S2, S3, S4, S5, or S6. For example, the orientations of the scFvs to the linker can vary. In one nucleic acid construct, a first scFv can be in the first cassette (i.e., before the linker), and a second scFv can be in the second cassette (i.e., after the linker). Alternatively, the first scFv can be in the second cassette, and the second scFv can be in the first cassette. Linkers of various lengths and flexibilities can be utilized as described herein. Different orientations of the two scFvs can influence binding.

The antigen recognition domain useful in constructing the chimeric B cell receptors, for example, scFvs directed toward a first antigen and/or a second antigen, can be synthesized, engineered, and/or produced using nucleic acids (e.g., DNA). The DNA encoding the antigen recognition domain can be cloned in frame to DNA encoding necessary chimeric B cell receptor elements such as, but not limited to, CD8 hinge regions, transmembrane domains, BCR associated proteins (such as CD79a and CD79b) and co-stimulatory domains of molecules of immunological interest such as, but not limited to, CD19 and CD20.

Binding domains other than scFv can also be used for predefined targeting of B cells, such as camelid single-domain antibody fragments or receptor ligands, antibody binding domains, antibody hypervariable loops or CDRs as non-limiting examples.

In an embodiment, the transmembrane domain further comprises a stalk region between said extracellular ligand-binding domain and said transmembrane domain. The term “stalk region” used herein can mean any oligo- or polypeptide that functions to link the transmembrane domain to the extracellular ligand-binding domain. In particular, stalk region are used to provide more flexibility and accessibility for the extracellular ligand-binding domain. A stalk region may comprise up to 300 amino acids, for example, 10 to 100 amino acids or, for example, 25 to 50 amino acids. Stalk region can be derived from all or part of naturally occurring molecules, such as from all or part of the extracellular region of CD8, CD4 or CD28, or from all or part of an antibody constant region (such as CH1, CH2, CH3, or both CH2 and CH3 for an IgG antibody, or CH1, CH2, CH3, CH4, or any combination thereof for an IgM antibody). For example, the stalk region can comprise an IgG (CH2-CH3), or a portion thereof. For example, the stalk region can comprise an IgG1 (CH2-CH3), an IgG2 (CH2-CH3), an IgG3 (CH2-CH3), an IgG₄ (CH2-CH3), or a portion thereof. In embodiments, the stalk region does not comprise a constant domain. In embodiments, the stalk region can be a synthetic sequence that corresponds to a naturally occurring stalk sequence, or can be an entirely synthetic stalk sequence. In an embodiment said stalk region is a part of human CD8 alpha chain.

The signal transducing domain or intracellular signaling domain of the chimeric B cell receptor of the invention can be responsible for intracellular signaling following the binding of extracellular ligand binding domain to the target resulting in the activation of the immune cell and immune response. In other words, the signal transducing domain can be responsible for the activation of at least one of the normal function of the B cell in which the chimeric B cell receptor is expressed. Thus, the term “signal transducing domain” can refer to the portion of a protein which directs the cell to perform a specialized function, such as early activation of Lyn and Syk and late activation of NFAT and NFκB as examples. In embodiments, the signal tranducing domain or intracellular signaling domain comprises all or part of CD79a and/or CD79b, which contains ITAMs to amplify signaling of the chimeric B cell receptor (BCR).

In embodiments, the signal transduction domain can comprise two distinct classes of cytoplasmic signaling sequence, those that initiate antigen-dependent primary activation, and those that act in an antigen-independent manner to provide a secondary or co-stimulatory signal. Primary cytoplasmic signaling sequence can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Examples of ITAM that can be used in the invention can include as non limiting examples those derived from TCR zeta, FcR gamma, FcR beta, FcR epsilon, CD3 gamma, CD3 delta, CD3 epsilon, CDS. CD22, CD79a, CD79b and CD66d.

The chimeric B cell receptor can comprise native transmembrane and/or intracellular domains. In native B cells, engagement of the B cell receptor leads to rapid tyrosine phosphorylation of the intracellular domains and calcium ion polarization, resulting in downstream activation of NFAT and NF-kB. Using the NFAT/NF-kB response elements to drive expression of our secreted proteins, we have designed an inducible expression system that will be activated by binding of the surface engineered BCR and the subsequent downstream signaling pathways.

In embodiments, the intracellular signaling domain of the BCR comprises a co-stimulatory signal molecule. In embodiments the intracellular signaling domain contains 2, 3, 4 or more co-stimulatory molecules in tandem. A co-stimulatory molecule can be a cell surface molecule other than an antigen receptor or their ligands that is required for an efficient immune response.

“Co-stimulatory ligand” can refer to a molecule that specifically binds a cognate co-stimulatory molecule on a cell, thereby providing a signal which, in addition to the primary signal provided by, for instance, binding of a ligand to the BCR, mediates a B cell response, including, but not limited to, proliferation, activation, differentiation and the like. A co-stimulatory ligand can include but is not limited to CD7, B7-1 (CD80), B7-2 (CD86), PD-L1, PD-L2, 4-1BBL, OX40L, inducible costimulatory ligand (ICOS-L), intercellular adhesion molecule (ICAM, CD30L, CD40, CD70, CD83, HLA-G, MICA, MI CB, HVEM, lymphotoxin beta receptor, 3/TR6, ILT3, ILT4, an agonist or antibody that binds Toll ligand receptor and a ligand that specifically binds with B7-H3. A co-stimulatory ligand can also encompass, inter alia, an antibody that specifically binds with a co-stimulatory molecule present on a B cell, such as but not limited to, CD27, CD28, 4-IBB, OX40, CD30, CD40, PD-1, ICOS, lymphocyte function-associated antigen-1 (LFA-1), CD2, CD7, LTGHT, NKG2C, B7-H3, a ligand that specifically binds with CD83.

A “co-stimulatory molecule” can refer to the cognate binding partner on a B-cell that specifically binds with a co-stimulatory ligand, thereby mediating a co-stimulatory response by the cell, such as, but not limited to proliferation, activation, differentiation, and the like. Co-stimulatory molecules can include, but are not limited to an MHC class 1 molecule, BTLA and Toll ligand receptor. Examples of costimulatory molecules include CD19, CD21, CD27, CD28, CD8, CD81, 4-1BB (CD137), OX40, CD30, CD40, PD-1, ICOS, lymphocyte function-associated antigen-I (LFA-1), CD2, CD7, LIGHT, NKG2C, B7-H3, TRL7/9, and a ligand that specifically binds with CD83 and the like. See, for example, Mongini, Patricia K A, and John K. Inman. “Cytokine dependency of human B cell cycle progression elicited by ligands which coengage BCR and the CD21/CD19/CD81 costimulatory complex.” Cellular immunology 207.2 (2001): 127-140.

In another embodiment, said signal transducing domain is a TNFR-associated Factor 2 (TRAF2) binding motifs, intracytoplasmic tail of costimulatory TNFR member family. Cytoplasmic tail of costimulatory TNFR family member contains TRAF2 binding motifs consisting of the major conserved motif (P/S/A)X(Q/E)E) or the minor motif (PXQXXD), wherein X is any amino acid. TRAP proteins are recruited to the intracellular tails of many TNFRs in response to receptor trimerization.

The distinguishing features of appropriate transmembrane polypeptides comprise the ability to be expressed at the surface of an immune cell, in particular B cells, and to interact together for directing cellular response of immune cell against a predefined target cell. The different transmembrane polypeptides of the chimeric B cell receptor comprising an extracellular ligand-binding domain and/or a signal transducing domain interact together to take part in signal transduction following the binding with a target ligand and induce an immune response. The transmembrane domain can be derived either from a natural or from a synthetic source. The transmembrane domain can be derived from any membrane-bound or transmembrane protein.

The term “a part of” used herein can refer to any subset of the molecule, that is a shorter peptide. Alternatively, amino acid sequence functional variants of the polypeptide can be prepared by mutations in the DNA which encodes the polypeptide. Such variants or functional variants include, for example, deletions from, or insertions or substitutions of, residues within the amino acid sequence. Any combination of deletion, insertion, and substitution may also be made to arrive at the final construct, provided that the final construct possesses the desired activity, especially to exhibit a specific anti-target cellular immune activity. The functionality of the chimeric B cell receptor of the invention within a host cell is detectable in an assay suitable for demonstrating the signaling potential of said chimeric B cell receptor upon binding of a particular target. Such assays are available to the skilled person in the art. For example, this assay allows the detection of a signaling pathway, triggered upon binding of the target, such as an assay involving measurement of tyrosine phosphorylation of the intracellular domain, or downstream activation of NFAT and NFκB activation.

In embodiments, polyclonal CASS B cells can be made by transducing cells with two lentiviruses (or two adeno-associated viruses) coding for different BCRs and sort for double transduced cells. Alternatively, polyclonal CASS B cells can be made by generating BCR library pools and using these plasmids to generate lentivirus (or adeno-associated virus) encoding a pool of BCRs. In this case one would get a population of CASS B cells that express different BCRs, and some of them could have undergone multiple transduction events and therefore display multiple BCRs on the surface of a single cell.

### Cells

Embodiments of the disclosure include B cells that express a chimeric B cell receptor. The cell can be of any kind, including an immune cell that can express the chimeric B cell receptor for a therapy (i.e., cancer therapy or infectious disease therapy) or a cell, such as a bacterial cell, that harbors an expression vector that encodes the chimeric B cell receptor. As used herein, the terms “cell,” “cell line,” and “cell culture” can be used interchangeably. All of these terms also include their progeny, which is any and all subsequent generations. For example, all progenies need not be identical due to deliberate or inadvertent mutations. In the context of expressing a heterologous nucleic acid sequence, “host cell” can refer to a eukaryotic cell that can replicate a vector and/or expressing a heterologous gene encoded by a vector. A host cell can, and has been, used as a recipient for vectors. A host cell can be “transfected”, “transformed” or “transduced” which can refer to a process by which exogenous nucleic acid is transferred or introduced into the host cell. A transformed cell includes the primary subject's cell and its progeny. As used herein, the terms “engineered” and “recombinant” cells or host cells can refer to a cell into which an exogenous nucleic acid sequence, such as, for example, a vector, has been introduced. Therefore, recombinant cells are distinguishable from naturally occurring cells which do not contain a recombinantly introduced nucleic acid. In embodiments of the invention, a host cell is a B cell.

Some vectors can employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

The cells can be autologous cells, syngeneic cells, allogenic cells and even in some cases, xenogeneic cells.

In many situations one wishes to be able to kill the modified B cells, such as when one wishes to terminate the treatment, if cells become neoplastic, in research where the absence of the cells after their presence is of interest, or other event. For this purpose one can provide for the expression of certain gene products in which one can kill the modified cells under controlled conditions, such as inducible suicide genes.

### Armed CASS B-Cells

The invention further includes genetically engineered CASS B cells that are modified to secrete one or more polypeptides. Such CASS B cells can be referred to as factories, CASS B cell factories, armed CASS B cells or immune restoring (IR) CASS B cells.

The term “polypeptide” can encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, can refer to “polypeptide” herein, and the term “polypeptide” can be used instead of, or interchangeably with any of these terms. “Polypeptide” can also refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced by recombinant technology, but is not necessarily translated from a designated nucleic acid sequence. It can be generated in any manner, including by chemical synthesis. As to amino acid sequences, one of skill in the art will readily recognize that individual substitutions, deletions or additions to a nucleic acid, peptide, polypeptide, or protein sequence which alters, adds, deletes, or substitutes a single amino acid or a small percentage of amino acids in the encoded sequence is collectively referred to herein as a “conservatively modified variant”. In some embodiments the alteration results in the substitution of an amino acid with a chemically similar amino acid. Conservative substitution tables providing functionally similar amino acids are well known in the art.

In embodiments, the polypeptide can be an antibody or fragment thereof, or a cytokine.

As used herein, an “antibody” or “antigen-binding polypeptide” can refer to a polypeptide or a polypeptide complex that specifically recognizes and binds to an antigen. By “specifically binds” or “immunoreacts with” is meant that the antibody reacts with one or more antigenic determinants of the desired antigen and does not react with other polypeptides. An antibody can be a whole antibody and any antigen binding fragment or a single chain thereof. For example, “antibody” can include any protein or peptide containing molecule that comprises at least a portion of an immunoglobulin molecule having biological activity of binding to the antigen.

As described herein, the terms “antibody fragment” or “antigen-binding fragment” is a portion of an antibody such as F(ab′)2, F(ab)2, Fab′, Fab, Fv, scFv and the like. Regardless of structure, an antibody fragment binds with the same antigen that is recognized by the intact antibody. The term “antibody fragment” can include aptamers (such as spiegelmers), minibodies, and diabodies. The term “antibody fragment” can also include any synthetic or genetically engineered protein that acts like an antibody by binding to a specific antigen to form a complex. Antibodies, antigen-binding polypeptides, variants, or derivatives described herein include, but are not limited to, polyclonal, monoclonal, multispecific (e.g. bi-specific), human, humanized or chimeric antibodies, mosaic antibodies, single chain antibodies, epitope-binding fragments, e.g., Fab, Fab′ and F(ab′) 2, Fd, Fvs, single-chain Fvs (scFv), single-chain antibodies, dAb (domain antibody), minibodies, disulfide-linked Fvs (sdFv), fragments comprising either a VL or VH domain, fragments produced by a Fab expression library, nanobodies obtained from camelids, and anti-idiotypic (anti-Id) antibodies.

In embodiments, the antibody secreted by the CASS B cell is a checkpoint blockade antibody. The term “checkpoint blockade antibody” can refer to an antibody that inhibits an immune checkpoint. When stimulated, key regulators of the immune system suppress the immune system's response to an immune stimulus, such as a cancer cell. Checkpoint blockade antibodies can block inhibitory checkpoints, restoring immune system function, such as against the cancer cell. Checkpoint blockade antibodies include, but are not limited to anti-PD-1, anti-PDL-2 and anti-CTLA-4. Other antibodies that modulate the immune system such as anti-TGFb and tumor vasculature such as anti-VEFG are also viable candidates.

In embodiments, the antibody secreted by the CASS B cell can be specific for HA1, HA2, NA, or a spike protein. Exemplary antibody compositions (e.g., VH and/or VL sequences or fragments thereof) that are useful for the armed B cells as described herein include, but are not limited to the anti-influenza antibodies described in PCT/US2008/085876 and PCT/US2016/026800.

In embodiments, the antibody secreted by the CASS B cell is specific for TIGIT, CAIX, GITR, PD-L1, PD-L2, PD-1, CCR4, CTLA-4, VISTA, CD70, PD-1, TIM-3, LAG-3, CD40L, or CXCR4. For example, CASS B cell factories can secrete PD-L1 mAbs locally at the tumor site to restore the effective anti-cancer immunity and/or reverse T cell exhaustion.

Exemplary antibody compositions (e.g., VH and/or VL sequences or fragments thereof) that are useful for the design of armed B-cells as described herein include, but are not limited to:
anti-CAIX antibodies described in PCT/US2006/046350 and PCT/US2015/067178
anti-CXCR4 antibodies described in PCT/US20006/005691
anti-CCR4 antibodies described in PCT/US2008/088435, PCT/US2013/039744, and PCT/US2015/054202
anti-PD-L1 antibodies described in PCT/US2008/088435 and PCT/US2020/062815
anti-PD-1 antibodies described in PCT/US2020/037791 and PCT/US2020/037781
anti-GITR antibodies described in PCT/US2017/043504
anti-claudin-4 antibodies described in PCT/US2019/022272
anti-MUC1 antibodies described in PCT/US2020/037783
anti-TIGIT antibodies described in U.S. provisional patent application 63/242,992
anti-IGHV 1-69 antibodies described in PCT/US2011/038970
anti-influenza antibodies described in PCT/US2008/085876 and PCT/US2016/026800
(each of the applications which are incorporated herein by reference in their entireties).

For exemplary antibodies to treat cancer, see also Yasunaga, Masahiro. Seminars in cancer biology. Vol. 64. Academic Press. 2020.

In embodiments, the antibody is a bi-specific antibody. For example, in embodiments, the antibody comprises a modular tetrameric/tetravalent bi-specific antibody as described in WO 2018/071913, which is incorporated by reference herein in its entirety. For example, the tetravalent bi-specific antibody is a dimer of a bi-specific scFv fragment including a first binding site for a first antigen, and a second binding site for a second antigen. For example, the bi-specific antibody can be specific for PD-1 and CTLA4, PD-1 and TIGIT, TIGIT and CCR4, or PD-1 and CCR4. The two binding sites can be joined together via a linker domain. In embodiments, the scFv fragment is a tandem scFv, the linker domain includes an immunoglobulin hinge region (e.g., an IgG₁, an IgG₂, an IgG₃, and an IgG₄ hinge region) amino acid sequence. In embodiments, the immunoglobulin hinge region amino acid sequence can be flanked by a flexible linker amino acid sequence, e.g., having the amino acid sequence (GGGS)ₓ₁₋₆ (SEQ ID NO: 1), (GGGGS)ₓ₁₋₆ (SEQ ID NO: 2), and GSAGSAAGSGEF (SEQ ID NO: 3). In embodiments, the linker domain includes at least a portion of an immunoglobulin Fc domain, e.g., an IgG₁, an IgG₂, an IgG₃, and an IgG₄ Fc domain. The at least a portion of the immunoglobulin Fc domain can be a CH2 domain. The Fc domain can be linked to the C-terminus of an immunoglobulin hinge region (e.g., an IgG₁, an IgG₂, an IgG₃, and an IgG₄ hinge region) amino acid sequence. The linker domain can include a flexible linker amino acid sequence (e.g., (GGGS)ₓ₁₋₆ (SEQ ID NO: 1), (GGGGS)ₓ₁₋₆ (SEQ ID NO: 2), and GSAGSAAGSGEF (SEQ ID NO: 3)) at one terminus or at both termini.

In embodiments, the cytokine secreted by the CASS B cell can be IL-12, IL-15, IL-18, IL-2, IL-7, CD40-L, or BAFF, or can be cytokine receptor/Fc fusion proteins.

Embodiments of the armed CASS B cells can comprise gene expression vectors which co-express multiple ORFs. The multiple ORFs can be separated by a linker, such as an internal ribosome entry site (IRES) or the 2A family of peptides. The 2A peptides are short (˜18-25 aa) peptides derived from viruses. The 2A peptides can be referred to as “self-cleaving”peptides, which will produce multiple proteins from the same transcript. 2A peptides function by making the ribosome skip the synthesis of the glycine and proline peptide bond at the C-terminal end of the 2A element, causing separation between the end of the 2A sequence and downstream peptide. As a result, the upstream protein will have a few extra 2A residues added to its C terminus while the downstream protein will have an extra proline added to its N terminus. There are four 2A peptides, P2A, T2A, E2A and F2A, that are derived from four different viruses.

In embodiments, the secretable polypeptide can be expressed from a second expression construct, which can be in the same DNA vector as that which encodes the chimeric B cell receptor (e.g. the antigen-recognition domain). The second express cassette, which can be used to encode the secretable polypeptide (i.e., antibody or cytokine), and can be cloned either before or after the linker (e.g., IRES or 2A family of peptides).

In embodiments, the second expression cassette encoding the secretable polypeptide can comprise a response element. A “response element” can refer to a portion of a gene which must be present in order for that gene to respond to some hormone or other stimulus. In embodiments, the response element is an inducible response element. For example, the response elements can be NFAT and/or NF-kB response elements.

In an embodiment, for example, the second expression cassette was inserted after the syn-BCR plasmid with multiple NFAT and/or NF-kB response elements and a minimal IL2/IL8 promoter upstream of a secreted protein (Ab or other protein). In native B cells, engagement of the BCR leads to rapid tyrosine phosphorylation of the IC domains and calcium ion polarization, resulting in downstream activation of NFAT and NF-kB. Using the NFAT/NF-kB response elements to drive expression of our secreted proteins, we have designed an inducible expression system that will be activated by antigens expressed on cancer cells. This creates a targeted and inducible delivery system that will only secrete its therapeutic payload when activated by tumor cells, resulting in a localized area of consistent and high immunomodulatory Ab/protein concentration centered around the tumor. This can reverse the suppressive nature of the tumor microenvironment, leading to improved outcomes and tumor elimination while decreasing on-target/off tumor side effects often seen by systemic delivery of therapeutic antibodies and cytokines.

In embodiments, the expression cassette can further comprise a post-transcriptional response element which, when transcribed, creates a tertiary structure enhancing expression. For example, the post-transcriptional response element can be WPRE.

Introduction of Constructs into B Cells

Expression vectors that encode the chimeric B cell receptors can be introduced as one or more DNA molecules or constructs, where there may be at least one marker that will allow for selection of host cells that contain the construct(s).

The constructs can be prepared in conventional ways, where the genes and regulatory regions can be isolated, as appropriate, ligated, cloned in an appropriate cloning host, analyzed by restriction or sequencing, or other convenient means. For example, using PCR, individual fragments including all or portions of a functional unit can be isolated, where one or more mutations may be introduced using “primer repair”, ligation, in vitro mutagenesis, etc., as appropriate. The construct(s) once completed and demonstrated to have the appropriate sequences may then be introduced into the B-cell by any convenient means. The constructs may be integrated and packaged into non-replicating, defective viral genomes like Adenovirus, Adeno-associated virus (AAV), or Herpes simplex virus (HSV) or others, including retroviral vectors or lentiviral vectors, for infection or transduction into cells. The constructs can include viral sequences for transfection. Alternatively, the construct can be introduced by fusion, electroporation, biolistics, transfection, lipofection, or the like. The host cells can be grown and expanded in culture before introduction of the construct(s), followed by the appropriate treatment for introduction of the construct(s) and integration of the construct(s). The cells are then expanded and screened by virtue of a marker present in the construct. Various markers that can be used successfully include hprt, neomycin resistance, thymidine kinase, hygromycin resistance, etc.

In some instances, one can have a target site for homologous recombination, where a construct be integrated at a particular locus. For example, one can knock-out an endogenous gene and replace it (at the same locus or elsewhere) with the gene encoded for by the construct using materials and methods as are known in the art for homologous recombination. For homologous recombination, one may use either .OMEGA. or O-vectors. See, for example, Thomas and Capecchi, Cell (1987) 51, 503-512; Mansour, et al., Nature (1988) 336, 348-352; and Joyner, et al., Nature (1989) 338, 153-156. Further, plasmid-based methods that induce double strand breaks have used homologous recombination for genome engineering. Zinc finger nucleases (ZFNs), TAL effector nucleases (TALENs), and CRISPR can all direct a nuclease to cause a specific double strand break.

The constructs can be introduced as a single DNA molecule encoding at least the CAR and optionally another gene, or different DNA molecules having one or more genes. Other genes include genes that encode therapeutic molecules or suicide genes, for example. The constructs may be introduced simultaneously or consecutively, each with the same or different markers.

Vectors containing useful elements such as bacterial or yeast origins of replication, selectable and/or amplifiable markers, promoter/enhancer elements for expression in prokaryotes or eukaryotes, etc. that may be used to prepare stocks of construct DNAs and for carrying out transfections are well known in the art, and many are commercially available.

### Methods of Treatment

Aspects of the disclosure are directed towards methods of preventing or treating a subject afflicted with a disease or disorder by administering a CASS B cell as described herein. In embodiments, the method comprises administering to a subject afflicted with or at risk of a disease or disorder a therapeutically effective amount of a CASS B cell as described herein. Therapeutically effective amounts can depend on the severity and course of the disease or disorder, previous therapy, the subject's health status, weight, and response to the drugs, and the judgment of the treating physician.

“Treatment” and “treating” can refer to the management and care of a subject for the purpose of combating a condition, disease or disorder, such as a cancer, or an infectious disease, in any manner in which one or more of the symptoms of a disease or disorder are ameliorated or otherwise beneficially altered. The term can include the full spectrum of treatments for a given condition from which the patient is suffering, such as administration of the active compound for the purpose of: alleviating or relieving symptoms or complications: delaying the progression of the condition, disease or disorder; curing or eliminating the condition, disease or disorder; and/or preventing the condition, disease or disorder, wherein “preventing” or “prevention” can refer to the management and care of a patient for the purpose of hindering the development of the condition, disease or disorder, and includes the administration of the active compounds to prevent or reduce the risk of the onset of symptoms or complications.

An “individual” or “subject” can be a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

The CASS B cells according to the disclosure can be used for preventing or treating a disease or disorder (e.g., cancer or an infection) in a subject in need thereof. In another embodiment, said isolated cell according to the invention can be used in the manufacture of a medicament for treatment of a cancer or infections (such as viral infections) in a patient in need thereof.

Embodiments can rely on methods for treating patients in need thereof, said method comprising at least one of the following steps: (a) providing a CASS B cell according to the invention and (b) administrating the cells to said patient.

Said treatment can be ameliorating, curative or prophylactic. It can be either part of an autologous immunotherapy or part of an allogenic immunotherapy treatment. By autologous, it is meant that cells, cell line or population of cells used for treating patients are originating from said patient or from a Human Leucocyte Antigen (HLA) compatible donor. By allogeneic is meant that the cells or population of cells used for treating patients are not originating from said patient but from a donor.

The invention is suited for allogenic immunotherapy, insofar as it allows the transformation of B-cells, obtained from donors, into non-alloreactive cells. This can be done under standard protocols and reproduced as many times as needed. The resulted modified B cells can be pooled and administrated to one or several patients, being made available as an “off the shelf” therapeutic product.

Cells that can be used with the disclosed methods are described in herein. Said treatment can be used to treat patients diagnosed with a disease or disorder.

Embodiments as described herein can modulate the immune system so as to treat the subject afflicted with a disease or disorder. “Modulating” can refer to up-regulation, induction, stimulation, potentiation, and/or relief of inhibition, as well as inhibition, attenuation and/or down-regulation or suppression. In embodiments, the activity of the subject's immune system is modulated.

Embodiments as described herein can be administered to a subject in combination with one or more therapies against a disease or disorder.

### Methods of Treating Cancer

Embodiments comprise methods of treating a subject afflicted with cancer. The term “cancer” can refer to the spectrum of pathological symptoms associated with the initiation or progression, as well as metastasis, of malignant tumors. The term “tumor” can refer to a new growth of tissue in which the multiplication of cells is uncontrolled and progressive. In embodiments, the tumor can be a malignant tumor, one in which the primary tumor has the properties of invasion or metastasis or which shows a greater degree of anaplasia than do benign tumors. Thus, “treatment of cancer” or “treating cancer” can refer to an activity that prevents, alleviates or ameliorates any of the primary phenomena (initiation, progression, metastasis) or secondary symptoms associated with the disease.

An emerging mechanism associated with the progression of tumors is the immune checkpoint pathway, which include cellular interactions that prevent excessive activation of T cells under normal conditions, allowing T cell function in a self-limited manner. As an evasion mechanism, many tumors are able to stimulate the expression of immune checkpoint molecules, resulting in an anergic phenotype of T cells that cannot restrain tumor progression. For example, emerging clinical data highlight the importance of one inhibitory ligand and receptor pair as an immune checkpoint: the programmed death-ligand 1 (PD-L1; B7-H1 and CD274) and programmed death receptor-1 (PD-1: CD279), in preventing killing of cancer cells by cytotoxic T-lymphocytes. PD1 receptor is expressed by many cell types like T cells, B cells, Natural Killer cells (NK) and host tissues. Tumors and Antigen-presenting cells (APC) expressing PD-L1 can block T cell receptor (TCR) signaling of cytotoxic T-lymphocytes through binding to receptor PD-1, decreasing the production of cytokines and T cell proliferation. PD-L1 overexpression can be found in many tumor types and may also mediate an immunosuppressive function through its interaction with other proteins, including CD80 (B7.1), blocking its ability to activate T cells through binding to CD28.

Genetic engineering of human B cells to express tumor-directed chimeric B cell receptors can produce antitumor effector cells that bypass tumor immune escape mechanisms that are due to abnormalities in protein-antigen processing and presentation. Moreover, these transgenic receptors can be directed to tumor-associated antigens that are not protein-derived.

For example, aspects of the disclosure are directed towards methods of killing a cancer cell, such as a renal cancer cell.

Aspects of the disclosure are further directed towards methods of stopping or reducing progression or promoting regression of a cancer in a subject.

Still further, aspects of the disclosure are directed towards a method of reducing cellular proliferation of a cancer cell in a subject.

“Cancer” and “cancerous” can refer to or describe, for example, the physiological condition in mammals that is characterized by unregulated cell growth. Examples of cancer include but are not limited to, carcinoma, lymphoma, chronic lymphocytic leukemia, non-small cell lung cancer, clear cell renal carcinoma, mesothelioma, blastoma, sarcoma, and leukemia. More particular examples of such cancers include squamous cell cancer, small cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer. For example, the cancer is renal cell carcinoma, such as ccRCC.

The subject can be afflicted with cancer such as liquid cancers (i.e., blood cancers) and/or solid cancers (i.e., tumors). The cancer can be benign or malignant, and can be one that is influenced by the immune system.

Cancers that may be treated include tumors that are not vascularized, or not yet substantially vascularized, as well as vascularized tumors. The cancers may comprise nonsolid tumors (such as hematological tumors, for example, leukemias and lymphomas) or may comprise solid tumors. Types of cancers to be treated with the CARs of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

In cancer, the normal intercellular interactions in tissues are disrupted, and the tumor microenvironment evolves to accommodate the growing tumor. The tumor microenvironment (TME) can refer to the cellular environment in which a tumor exists, including components such as surrounding blood vessels, immune cells, fibroblasts, bone marrow-derived inflammatory cells, lymphocytes, signaling molecules and the extracellular matrix (ECM). Tumor microenvironment is complex and is heavily influenced by immune system.

This invention provides CASS B cell therapy for the treatment or prevention of cancer. The secretion of a mono, bi-, or tri-specific minibody, antibody or minibody/antibody fusion protein or a cytokine by the CASS B cell at the tumor site could provide additional benefit by altering (i.e., modulating) the immune-repressive tumor microenvironment. For example, the microenvironment surround the cancer cell and/or tumor can be modulated such that the microenvironment-dependent immune suppression is reduced so as to modulate (or allow) the immune system to kill tumor cells.

In embodiments of the invention, methods of the invention for clinical aspects are combined with other agents effective in the treatment of hyperproliferative disease, such as anti-cancer agents. An “anti-cancer” agent can negatively affect cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. For example, these other compositions would be provided in a combined amount effective to kill or inhibit proliferation of the cell. This process may involve contacting the cancer cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with other therapies. In one embodiment, cell therapy could be used similarly in conjunction with chemotherapeutic, radiotherapeutic, or immunotherapeutic intervention, as well as pro-apoptotic or cell cycle regulating agents.

Alternatively, the present inventive therapy can precede or follow the other agent treatment by intervals ranging from minutes to weeks. In embodiments where the other agent and present invention are applied separately to the individual, one would generally ensure that a significant period of time did not expire between the time of each delivery, such that the agent and inventive therapy would still be able to exert an advantageously combined effect on the cell. In such instances, one may contact the cell with both modalities within about 12-24 h of each other (for example, within about 6-12 h of each other). In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

The treatment cycles would be repeated as necessary. For example, various standard therapies, as well as surgical intervention, may be applied in combination with the inventive cell therapy.

Cancer therapies also include a variety of combination therapies with both chemical and radiation-based treatments. Combination chemotherapies include, but are not limited to, for example, abraxane, altretamine, docetaxel, herceptin, methotrexate, novantrone, zoladex, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing and also combinations thereof. In specific embodiments, chemotherapy for the individual is employed in conjunction with the invention, for example before, during and/or after administration of the invention.

Other factors that cause DNA damage and have been used extensively include what are commonly known as .gamma.-rays, X-rays, and/or the directed delivery of radioisotopes to tumor cells. Other forms of DNA damaging factors are also useful such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

Immunotherapeutics rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy other than the inventive therapy described herein could thus be used as part of a combined therapy, in conjunction with the present cell therapy. The approach for combined therapy is discussed herein. For example, the tumor cell must bear some marker that is amenable to targeting, i.e., is not present on the majority of other cells. Many tumor markers exist and any of these may be suitable for targeting in the context of the present invention. Common tumor markers include PD-1, PD-L1, CTLA4, carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

In yet another embodiment, the secondary treatment is a gene therapy in which a therapeutic polynucleotide is administered before, after, or at the same time as the present invention clinical embodiments. A variety of expression products are encompassed within the invention, including inducers of cellular proliferation, inhibitors of cellular proliferation, or regulators of programmed cell death.

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). For example, the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

In some embodiments, other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor: interferon alpha, beta, and gamma: IL-2 and other cytokines; F42K and other cytokine analogs: or MIP-1, MIP-1beta, MCP-1, RANTES, and other chemokines. In some embodiments, upregulation of cell surface receptors or their ligands such as Fas/Fas ligand, DR4 or DR5/TRAIL would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion can also be used to improve the efficacy of the present invention. Examples of cell adhesion inhibitors are focal adhesion kinase (FAKs) inhibitors and Lovastatin. In some embodiments, other agents that increase the sensitivity of a hyperproliferative cell to apoptosis, such as the antibody c225, could be used in combination with the present invention to improve the treatment efficacy.

According to an embodiment of the invention, said treatment can be administrated into patients undergoing an immunosuppressive treatment. The present invention uses cells or population of cells, which have been made resistant to at least one immunosuppressive agent due to the inactivation of a gene encoding a receptor for such immunosuppressive agent. In this aspect, the immunosuppressive treatment should help the selection and expansion of the T-cells according to the invention within the patient.

In a further embodiment, the cell compositions as described herein can be administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAM PATH. In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery. Said modified cells obtained by any one of the methods described here can be used in a particular aspect of the invention for treating patients in need thereof against Host versus Graft (HvG) rejection and Graft versus Host Disease (GvHD); therefore in the scope of the present invention is a method of treating patients in need thereof against Host versus Graft (HvG) rejection and Graft versus Host Disease (GvHD) comprising treating said patient by administering to said patient an effective amount of modified cells comprising inactivated TCR alpha and/or TCR beta genes.

### Methods of Treating an Infectious Disease

Aspects of the invention are drawn towards methods of treating or preventing an infectious disease in a subject by administering to the subject a composition as described herein. The term “infectious disease” can refer to an organism (e.g. virus, fungi or bacteria) that is deleterious to its host. In some embodiments the agent is deleterious to a human host. An “anti-infectious disease” treatment refers to a treatment that prevents, ameliorates or eradicates the infectious disease and/or its disease-causing agent.

Examples of infectious diseases include without limitation, HIV, West Nile virus, hepatitis A, B, C, smallpox, tuberculosis, Vesicular Stomatitis Virus (VSV), Respiratory Syncytial Virus (RSV), human papilloma virus (HPV), SARS, influenza, coronavirus, Ebola, viral meningitis, herpes, anthrax, lyme disease, and E. coli, among others. See, for example, Pelegrin, Mireia, Mar Naranjo-Gomez, and Marc Piechaczyk. “Antiviral monoclonal antibodies: can they be more than simple neutralizing agents?.” Trends in microbiology 23.10 (2015): 653-665; Salazar, Georgina, et al. “Antibody therapies for the prevention and treatment of viral infections.” npj Vaccines 2.1 (2017): 1-12.

### Administration of Cells

The disclosure is particularly suited for allogenic immunotherapy, insofar as it allows the transformation of B cells, obtained from donors, into non-alloreactive cells. This can be done under standard protocols and reproduced as many times as needed. The resulted modified B cells can be pooled and administrated to one or several patients, being made available as an “off the shelf” therapeutic product.

Depending upon the nature of the cells, the cells can be introduced into a host organism, e.g. a mammal, in a wide variety of ways. The cells can be introduced at the site of the tumor, in specific embodiments, although in alternative embodiments the cells hone to the cancer or are modified to hone to the cancer. The number of cells that are employed will depend upon a number of circumstances, the purpose for the introduction, the lifetime of the cells, the protocol to be used, for example, the number of administrations, the ability of the cells to multiply, the stability of the recombinant construct, and the like. The cells may be applied as a dispersion, for example, being injected at or near the site of interest. The cells may be in a physiologically-acceptable medium.

In some embodiments, the cells are encapsulated to inhibit immune recognition and placed at the site of the tumor.

The cells can be administered as desired. Depending upon the response desired, the manner of administration, the life of the cells, the number of cells present, various protocols may be employed. The number of administrations will depend upon the factors described above at least in part.

The administration of the cells or population of cells according to the present invention can be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous or intralymphatic injection, or intraperitoneally. In one embodiment, the cell compositions of the present invention are administered by intravenous injection.

The administration of the cells or population of cells can consist of the administration of 104-109 cells per kg body weight, for example, 105 to 106 cells/kg body weight including all integer values of cell numbers within those ranges. The cells or population of cells can be administrated in one or more doses. In another embodiment, said effective amount of cells are administrated as a single dose. In another embodiment, said effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

It should be appreciated that the system is subject to many variables, such as the cellular response to the ligand, the efficiency of expression and, as appropriate, the level of secretion, the activity of the expression product, the particular need of the patient, which may vary with time and circumstances, the rate of loss of the cellular activity as a result of loss of cells or expression activity of individual cells, and the like. Therefore, for each individual patient, even if there were universal cells which could be administered to the population at large, each patient would be monitored for the proper dosage for the individual, and such practices of monitoring a patient are routine in the art.

### Nucleic Acid-Based Expression Systems

The chimeric B cell receptors of the disclosure can be expressed from an expression vector. Recombinant techniques to generate such expression vectors are well known in the art.

DNA constructs, which can also be referred to as “DNA vectors”, as described herein, can be cloned into a vector which will be used to transduce and produce CASS B cells that secrete polypeptides and/or fragments thereof. For example, DNA constructs can be cloned into a lentiviral vector for production of lentivirus, which will be used to transduce and produce chimeric-antigen receptor T-cells that secrete a mono, bi- or tri-specific immune-modulating antibody/minibody and/or antibody-fusion protein at the tumor site. For example, DNA constructs can be cloned into an adeno-associated viral vector for production of adeno-associated virus, which will be used to transduce and produce chimeric-antigen receptor T-cells that secrete a mono, bi- or tri-specific immune-modulating antibody/minibody and/or antibody-fusion protein at the tumor site.

In embodiments, the DNA construct can comprise a nucleic acid encoding one or more polypeptides, such as a chimeric B cell receptor and/or a secreted polypeptide.

### Vectors

The term “vector” can refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” refers to any type of genetic construct comprising a nucleic acid coding for an RNA that can be transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and translation of an operably linked coding sequence in a particular host cell. In addition to control sequences that govern transcription and translation, vectors and expression vectors may contain nucleic acid sequences that serve other functions as well and are described infra.

### Promoters and Enhancers

A “promoter” is a control sequence that is a region of a nucleic acid sequence at which initiation and rate of transcription are controlled. It can contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter comprises a sequence that functions to position the start site for RNA synthesis. The best-known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. These are located in the region 30 110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter can be one naturally associated with a nucleic acid sequence, as may be obtained by isolating the 5 prime′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination could also be used to drive expression. Use of a T3, T7 or SP6 cytoplasmic expression system is another embodiment. Eukaryotic cells can support cytoplasmic transcription from certain bacterial promoters if the appropriate bacterial polymerase is provided, either as part of the delivery complex or as an additional genetic expression construct.

The identity of tissue-specific promoters or elements, as well as assays to characterize their activity, is well known to those of skill in the art.

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily determine this and providing the necessary signals.

In certain embodiments of the disclosure, the use of internal ribosome entry sites (IRES) elements are used to create multigene, or polycistronic, messages, and these may be used in the invention.

Vectors can include a multiple cloning site (MCS), which is a nucleic acid region that contains multiple restriction enzyme sites, any of which can be used in conjunction with standard recombinant technology to digest the vector. “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to allow exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

Splicing sites, termination signals, origins of replication, and selectable markers may also be employed.

### Plasmid Vectors

In certain embodiments, a plasmid vector can be used to transform a host cell. Plasmid vectors containing replicon and control sequences which are derived from species compatible with the host cell are used in connection with these hosts. The vector ordinarily carries a replication site, as well as marking sequences which can provide phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters that can be used by the microbial organism for expression of its own proteins.

In addition, phage vectors containing replicon and control sequences that are compatible with the host microorganism can be used as transforming vectors in connection with these hosts. For example, the phage lambda GEM™ 11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S transferase (GST) soluble fusion proteins for later purification and separation or cleavage. Other suitable fusion proteins are those with galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, for example, between 2 and 24 h, the cells are collected by centrifugation and washed to remove residual media.

### Viral Vectors

The ability of certain viruses to infect cells or enter cells via receptor mediated endocytosis, and to integrate into host cell genome and express viral genes stably and efficiently have made them attractive candidates for the transfer of foreign nucleic acids into cells (e.g., mammalian cells). Components of the present invention may be a viral vector that encodes one or more CARs of the invention. Non-limiting examples of virus vectors that may be used to deliver a nucleic acid of the present invention are described below.

### Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell specific construct that has been cloned therein. Knowledge of the genetic organization or adenovirus, a 36 kb, linear, double stranded DNA virus, allows substitution of large pieces of adenoviral DNA with foreign sequences up to 7 kb (Grunhaus and Horwitz, 1992).

### AAV Vectors

The nucleic acid can be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno associated virus (AAV) is an attractive vector system for use in the cells of the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; Mclaughlin et al., 1988). Details concerning the generation and use of rAAV vectors are described in U.S. Pat. Nos. 5,139,941 and 4,797,368, each incorporated herein by reference.

### Retroviral Vectors

Retroviruses are useful as delivery vectors because of their ability to integrate their genes into the host genome, transferring a large amount of foreign genetic material, infecting a broad spectrum of species and cell types and of being packaged in special cell lines (Miller, 1992).

In order to construct a retroviral vector, a nucleic acid (e.g., one encoding the sequence of interest) is inserted into the viral genome in the place of certain viral sequences to produce a virus that is replication defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors can infect non-dividing cells and can be used for both in vivo and ex vivo gene transfer and expression of nucleic acid sequences. For example, recombinant lentivirus can infect a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

### Other Viral Vectors

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

### Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A new approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

### Vector Delivery and Cell Transformation

Suitable methods for nucleic acid delivery for transfection or transformation of cells are known to one of ordinary skill in the art. Such methods include, but are not limited to, direct delivery of DNA such as by ex vivo transfection, by injection, and so forth. Through the application of techniques known in the art, cells may be stably or transiently transformed.

### Ex Vivo Transformation

Methods for transfecting eukaryotic cells and tissues removed from an organism in an ex vivo setting are known to those of skill in the art. Thus, cells or tissues may be removed and transfected ex vivo using nucleic acids of the present invention. In some embodiments, the transplanted cells or tissues may be placed into an organism. In other embodiments, a nucleic acid is expressed in the transplanted cells.

### Pharmaceutical Compositions

The invention provides for a therapeutic composition, or a “pharmaceutical composition” or “formulation” comprising a CASS B cell as described herein and a pharmaceutically acceptable carrier. The invention also provides for a therapeutic composition comprising a nucleic acid as described herein and a pharmaceutically acceptable carrier.

As used herein, a “pharmaceutical composition” or a “pharmaceutical formulation” can refer to a composition or pharmaceutical composition suitable for administration to a subject, such as a mammal, especially a human, and that refers to the combination of an active agent(s) (e.g., genetically engineered cell), or ingredient with a pharmaceutically acceptable carrier or excipient, making the composition suitable for diagnostic, therapeutic, or preventive use in vitro, in vivo. or ex vivo. According to the invention, a pharmaceutical composition can be sterile and free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, intravenous, buccal, rectal, parenteral, intraperitoneal, intradermal, intracheal, intramuscular, subcutaneous, inhalational and the like.

In embodiments, the pharmaceutical composition can contain components that ensure the viability of the CASS B cells therein. In particular, the cells can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996, which is incorporated herein in its entirety. Choice of the cellular excipient and any accompanying elements of the composition will be adapted in accordance with the device used for administration. For example, the composition can comprise a suitable buffer system to suitable pH, e.g., near neutral pH (e.g., phosphate or carbonate buffer system), and can comprise sufficient salt to ensure iso-osmotic conditions for the cells, i.e., preventing osmotic stress. For example, suitable solution for these purposes can be phosphate-buffered saline (PBS) as known in the art. Further, the composition can comprise a carrier protein, e.g., albumin, which can increase the viability of the cells. To ensure exclusion of non-human animal material, the albumin can be of human origin (e.g., isolated from human material or produced recombinantly). Suitable concentrations of albumin are generally known.

Hence, pharmaceutical compositions according to the invention, and for use in accordance with the invention, can comprise a pharmaceutically acceptable excipient, carrier, buffer, preservative, stabilizer, anti-oxidant or other material well known to those skilled in the art. Such materials should be non-toxic and should not interfere with the activity of the cells or the nucleic acids. The precise nature of the carrier or other material will depend on the route of administration. The composition can include one or more of cytoprotective molecules. Such substances can render the cells independent of its environment.

A “pharmaceutically acceptable excipient,” “pharmaceutically acceptable diluent,” “pharmaceutically acceptable carrier,” or “pharmaceutically acceptable adjuvant” can refer to an excipient, diluent, carrier, and/or adjuvant that are useful in preparing a pharmaceutical composition that are generally safe, non-toxic and neither biologically nor otherwise undesirable, and include an excipient, diluent, carrier, and adjuvant that are acceptable for veterinary use and/or human pharmaceutical use. A pharmaceutically acceptable excipient, diluent, carrier and/or adjuvant as used in the specification and claims includes one and more such excipients, diluents, carriers, and adjuvants.

The invention also encompasses methods of producing said pharmaceutical compositions by mixing the cells and/or nucleic acids of the invention with one or more additional components as above. Liquid pharmaceutical compositions generally include a liquid carrier such as water, petroleum, animal or vegetable oils, mineral oil or synthetic oil. Physiological saline solution, tissue or cell culture media, dextrose or other saccharide solution or glycols such as ethylene glycol, propylene glycol or polyethylene glycol may be included.

The composition can be in the form of a parenterally acceptable aqueous solution, which is pyrogen-free and has suitable pH, isotonicity and stability. Those of relevant skill in the art are well able to prepare suitable solutions using, for example, isotonic vehicles such as Sodium Chloride, Ringer's Injection, or Lactated Ringer's Injection. A composition can be prepared using biological fluids, such as artificial cerebrospinal fluid. In a further aspect, the invention relates to an arrangement comprising a surgical instrument for administration of a composition at a site of tissue dysfunction or lesion, and further comprising the pharmaceutical composition as defined above, wherein the arrangement is adapted for administration of the pharmaceutical composition at the site of tissue dysfunction or lesion. For example, a suitable surgical instrument can be capable of injecting a liquid composition comprising the genetically engineered cells as described herein at the site of neural dysfunction or lesion. Cells can be implanted into a patient by any technique known in the art, including those described in Freed et al. 1997. Cell Transplant 6:201-202: Kordower et al. 1995. N Engl J Med 332:1 118-1 124: Freed et al. 1992. N Engl J Med 327:1549-1555: Tateishi-Yuyama, Eriko, et al. The Lancet 360.9331 (2002): 427-435: THOMA, CHRISTINE, et al. Nature medicine 3.3 (1997); Kondziolka, D., et al. Neurology 55.4 (2000): 565-569, the entire disclosure of each which are incorporated herein by reference.

### Method of Making a Population of Genetically Engineered B Cells

Embodiments of the invention are also drawn towards methods of making a population of genetically engineered B cells. For example, such methods comprise isolating a population of B cells from a subject, and transducing the population of B cells with a vector(s) as described herein, thereby producing a population of genetically engineered B cells.

The cells, such as B cells, into which a polynucleotide is to be introduced into can be obtained from sources such as the subjects themselves, donor subjects, or cell banks. For example, the cells can be harvested from a subject, as is the case with B cells that can be used for autologous transplantation.

In embodiments, the polynucleotide can be introduced into the cells by transduction, such as transfer by bacteriophages or viruses: transformation, such as uptake of naked DNA from outside of the cell: or microinjection.

As needed, positive and negative controls can be used. For example, positive controls for transduction efficiency can be empty plasmids lentiviral stocks carrying the mCherry, eGFP, or other fluorescent molecular tags, such as YFP, BFP, or RFP.

In embodiments, the method further comprises the step of activating the population of B cells prior to transduction.

In embodiments, the method can further comprise the step of culturing the population of genetically engineered B cells. Culturing cells can refer to the process of keeping cells in conditions appropriate for maintenance and/or growth, where conditions can refer to, for example, the temperature, nutrient availability, atmospheric CO₂ content and cell density in which the cells are kept. Cells can be cultured in vivo or in vitro. The appropriate culturing conditions for maintaining, proliferating, expanding and differentiating different types of cells are known to the skilled artisan. See, for example, Moffett, H. F. et al (2019). B cells engineered to express pathogen-specific antibodies protect against infection. Science Immunology, 4 (35).

### Kits of the Invention

Any of the compositions described herein may be comprised in a kit. In a non-limiting example, one or more cells for use in cell therapy and/or the reagents to generate one or more cells for use in cell therapy that harbors recombinant expression vectors may be comprised in a kit. The kit components are provided in suitable container means.

Some components of the kits may be packaged either in aqueous media or in lyophilized form. The container means of the kits can include at least one vial, test tube, flask, bottle, syringe or other container means, into which a component may be placed, and suitably aliquoted. Where there are more than one component in the kit, the kit also can contain a second, third or other additional container into which the additional components may be separately placed. However, various combinations of components may be comprised in a vial. The kits of the present invention also can include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. For example, the solvent may also be provided in another container means. The kits may also comprise a second container means for containing a sterile, pharmaceutically acceptable buffer and/or other diluent.

In particular embodiments of the invention, cells that are to be used for cell therapy are provided in a kit, and in some cases the cells are essentially the sole component of the kit. The kit may comprise reagents and materials to make the desired cell. In specific embodiments, the reagents and materials include primers for amplifying desired sequences, nucleotides, suitable buffers or buffer reagents, salt, and so forth, and in some cases the reagents include vectors and/or DNA that encodes a chimeric B cell receptor as described herein and/or regulatory elements therefor.

In particular embodiments, there are one or more apparatuses in the kit suitable for extracting one or more samples from an individual. The apparatus may be a syringe, scalpel, and so forth.

In some cases of the invention, the kit, in addition to cell therapy embodiments, also includes a second cancer therapy, such as chemotherapy, hormone therapy, and/or immunotherapy, for example. The kit(s) may be tailored to a particular cancer for an individual and comprise respective second cancer therapies for the individual.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Engineering Chimeric Antibody Signaling and Secreting (CASS) B Cells as a Targeted and Inducible Platform to Secrete Immunomodulatory Proteins at the Tumor Site

Membrane Ig constructs (synthetic BCRs) were designed by linking a scFv to a membrane tethered IgG or IgM hinge-CH2-CH3 with the native transmembrane and intracellular domains. A secondary cassette in was inserted after the syn-BCR plasmid with multiple NFAT and/or NF-kB response elements and a minimal IL2/IL8 promoter upstream of a secreted protein (Ab or other protein, for example a cytokine).

In native B cells, engagement of the BCR leads to rapid tyrosine phosphorylation of the intracellular domains and calcium ion polarization, resulting in downstream activation of NFAT and NF-kB. Using the NFAT/NF-kB response elements to drive expression of our secreted proteins, we have designed an inducible expression system that will be activated by antigens expressed on cancer cells. This creates a targeted and inducible delivery system that will only secrete its therapeutic payload when activated by tumor cells, resulting in a localized area of consistent and high immunomodulatory Ab/protein concentration centered around the tumor. This can reverse the suppressive nature of the tumor microenvironment, leading to improved outcomes and tumor elimination while decreasing on-target/off tumor side effects often seen by systemic delivery of therapeutic antibodies and cytokines.

Non-limiting examples include an anti-CAIX CASS B cell that would secrete anti-PD1/anti-CTLA4 bi-specific antibody, and an anti-mesothelin CASS B cell that would secrete an anti-PD1/anti-TIGIT bi-specific antibody. Non-limiting examples of other secreted antibodies include anti-CCR4, anti-PDL1, anti-VEGF, anti-CAIX, anti-PD-1, anti-PD-L1, anti-PD-L2, anti-CTLA4, anti-TIGIT, anti-VISTA, anti-CD70, anti-TIM-3, anti-LAG-3, anti-CD40L, anti-CCR4, anti-GITR, or anti-CXCR4.

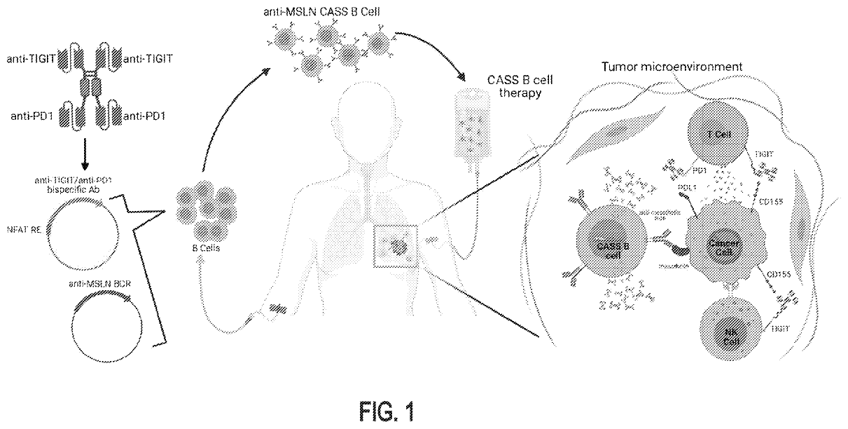

FIG. 1 refers to a CASS B cell schematic. This schematic uses separate plasmids for the syn-BCR and secreted Ab, however this can be combined into one plasmid also.

Current therapeutic methods include CAR T cells and systemic Ab delivery. CAR T cell therapy is associated with cytotoxicity. Unlike CAR-T cells, embodiments herein use a B cell, which will not lead to direct cytotoxicity of the tumor as seen with CAR T cells. Rather the genetically engineered B cells described herein focus on reversing the suppressive nature of the tumor microenvironment by secreting high levels of Ab/cytokines locally around the tumor. This will allow the rest of the immune system to destroy the tumor and also provide life-long protection against re-growth and metastasis. The addition of the inducible system is also a key component as this will decrease the serum concentration of our Abs, reducing off tumor/on target side effects.

Thus, embodiments described herein can be used to treat solid tumors, especially those that have very immunosuppressive tumor microenvironments. Embodiments described herein can also be used for the prevention and/or treatment of other indications, including infectious diseases.

### Example 2

### Engineered Chimeric Antibody Signaling and Secreting (CASS) B Cells to Achieve Cancer Cures

### Abstract

Immune checkpoint blockade inhibitors (CBI) and CAR T cells have revolutionized the way we treat cancer. While both of these therapies engage the patient's immune system, neither is able to proactively initiate an anti-tumor immune response and significant limitations exist in their scope of use and efficacy. To address this, described herein are Chimeric Antibody Secreting and Signaling (CASS) B cells, which express an engineered, tumor targeting B cell receptor and upon engagement, will secrete high levels of a dual-targeted bi-specific checkpoint blockade modulator antibody (e.g., dual-targeted bi-specific checkpoint inhibitor antibody) locally at the tumor site. As B cells also serve as professional antigen presenting cells, they can process and present antigens on MHC class II molecules, further enhancing immune cell recognition of the tumor and assisting in neoantigen spreading. A key component of immunologic memory, CASS B cells will simultaneously recruit a wide range of immune cells and reverse tumor infiltrating lymphocyte exhaustion, providing a robust and lifelong surveillance program protecting against tumor metastasis and recurrence. Non-small cell lung cancer (NSCLC) was chosen as a model to develop an anti-MSLN directed CASS B cell secreting an immunomodulatory anti-PD1/TIGIT bi-specific antibody (bsAb). Aim 1 will focus on the development of the CASS B cell platform and at the conclusion of this stage, we will have identified the three antibodies and optimized the signaling domains that comprise CASS B cells. In Aim 2, in vitro characterization and efficacy testing will be performed, resulting in a clear understanding of the linkage between CASS B cell activation and bsAb secretion, while providing critical analysis of CASS B cell efficacy in comparison to CAR T cells at both a functional and molecular level. Aim 3 will execute in vivo experiments using cell line derived and patient derived NSCLC models in humanized mice. Multiparameter flow cytometry, single cell RNA sequencing, and immunohistochemistry will provide a detailed assessment of the molecular and mechanistic efficacy of the immunomodulatory bsAb and the CASS B cell platform as a whole.

### Research Objectives

Without wishing to be bound by theory, we will develop a new type of combination cellular immunotherapy, Chimeric Antibody Secreting and Signaling (CASS) B cells. These B cells will express an engineered tumor associated antigen (TAA) targeting B cell receptor (BCR), and upon engagement, will secrete high levels of a checkpoint blockade modulator (e.g., dual-targeted bi-specific checkpoint inhibitor antibody) bi-specific antibody (bsAb) locally at the tumor site. This will allow for reversal of the immunosuppressive tumor microenvironment (TME) and restoration of the patient's natural innate and adaptive immunity to eliminate cancerous cells. In addition, unlike T cells, B cells act as professional antigen presenting (APC) cells and should enhance tumor cell recognition and assist in neoantigen spreading, thereby leading to both reversal of tumor infiltrating lymphocyte (TIL) exhaustion and induction of a broader and more robust anti-tumor immune response. For proof-of-principle studies we propose to engineer an anti-PD1/TIGIT bsAb secreting CASS B cell that targets mesothelin (MSLN) for the treatment of NSCLC¹⁻³.

The first objective focuses on the construction and optimization of the engineered anti-MSLN IgG-BCR, the anti-PD1/TIGIT bsAb to be delivered at the tumor site, and the inducible response element (RE) that drives bsAb expression. Panels of anti-MSLN, anti-PD1, and anti-TIGIT antibodies were identified by our lab and functional assays will identify lead candidates. Concurrent efforts will focus on the development of the inducible response element and optimization of B cell transduction conditions.

The second objective is the functional evaluation of the anti-MSLN CASS B cell in vitro. Activation assays will be used to quantify bsAb and cytokine secretion levels. Patient-derived organotypic tumor spheroids (PDOTS) will be used to evaluate CASS B cell efficacy and for molecular/mechanistic comparisons with CAR T cells via cytokine profiling, IHC, and single cell RNA sequencing (scRNAseq). Also, embodiments can use mesothelioma tumors for PDOTs.

The final objective will utilize HLA matched, humanized mice to generate cell line derived (CDX) and patient derived xenograft (PDX) models of NSCLC to test CASS B cell efficacy. Models will be paired with various analytical techniques (IHC, flow cytometry, scRNAseq) to further interrogate the effects of CASS B cell therapy on the surrounding TME.

### Background

Checkpoint blockade inhibitor (CBI) monoclonal antibodies (mAbs) and adoptive cellular therapies have had a transformative effect on cancer therapies, changing the focus from simply killing tumor cells to activating a patients natural anti-tumor immunity and reversing the immunosuppressive tumor microenvironment (TME). While these represent some of the most promising anti-cancer therapeutics to date, only a small subset of patients experience complete or durable responses and many experience immune related adverse events (irAE) of varying severity⁴⁻⁷. To counter this, combination CBI therapies such as anti-PD(L)1/anti-TIGIT have been tested and demonstrate significant promise in clinical trials⁸⁻¹¹. Another approach is the development of armored or immune restoring CAR T cells that are engineered to secrete an immunomodulatory payload directly at the tumor site, increasing the efficacy while decreasing the on-target/off-tumor side effects seen with systemic delivery^{12,13}.

In addition to T cells, various immune cells can be utilized for the creation of new CARs, including natural killer cells (NK-CAR) and macrophages (CAR-M) and what unites all of these cells is that they provide direct anti-tumor activity^{14,15}. A critical component of humoral immunity, B cells produce the antibodies on which the field of immunotherapy was originally developed. However, they possess no intrinsic cytotoxic capabilities and thus have been largely excluded from these advancements.

The Chimeric Antibody Secreting and Signaling (CASS) B cell platform will bring B cell research into the 21^{st} century by developing a unique B cell based cellular therapy that does not rely upon direct cytotoxicity but utilizes two intrinsic capabilities of B cells: the ability to secrete high levels of CBI antibodies to reverse the immunosuppressive TME and the ability to process and present antigens on MHC class II molecules, resulting in the recruitment of CD4+ T cells and allowing for enhanced tumor cell recognition and neoantigen spreading. While inducible, targeted delivery of the CBI will decrease irAEs, the ability to serve as a professional APC makes the CASS B cell platform unique in the cell therapy space, as CASS B cells have the power to initiate a robust anti-tumor response. The impact of this is further exemplified in work that demonstrated that MHC class II neoantigens played a key function in innate anti-tumor responses^{16,17}.

While this example focuses on MSLN+ targeted CASS B cells secreting an anti-PD1/TIGIT bsAb for NSCLC, the modular design of CASS B cells allow for easy targeting of other TAAs and the secreted payload can be adjusted to target the relevant immunologic axis, allowing for the adaptation of CASS B cells to a wide variety of cancers. As B cells are long lived and an important part of immunologic memory, CASS B cells continuously deliver the therapeutic payload at the tumor and after the primary tumor has been eradicated, provides a lifelong immunosurveillance system against metastasis and reoccurrence.

B cell engineering is a more recent accomplishment and has mainly focused on creating B cells that secrete neutralizing, anti-pathogenic antibodies against RSV and HIV²¹⁻²³. Unlike the cancer therapeutic proposed here, the B cells described in these works focus on systemic production of antibodies to neutralize viral infection and utilize CRISPR/Cas9 to insert the recombinant mAb into the Ig locus of the B cell. This has the added benefit of allowing the antibody to continue to undergo affinity maturation, a requirement for combating infectious diseases but not for targeting immune markers. The sum of these works indicate that, it is possible to manipulate a B cell to express an engineered antibody of choice in an inducible manor and that upon activation, these engineered B cells not only differentiate into Ab secreting cells, but also memory B cells providing long-term protection²⁴.

### Non-Limiting Examples of Research Plan

Aim 1: Engineering and optimization of CASS B cell constructs—The appropriate scFvs (anti-MSLN, PD1, TIGIT) will be selected for CASS B cell development followed by optimization of the signaling domains and inducible response element²⁵⁻²⁸. Protocols for high efficiency transduction and exponential expansion of primary B cells will also be optimized utilizing various lentivirus envelope proteins and culture conditions²⁹⁻³².

### Non-Limiting Examples of Experimental Design and Procedures

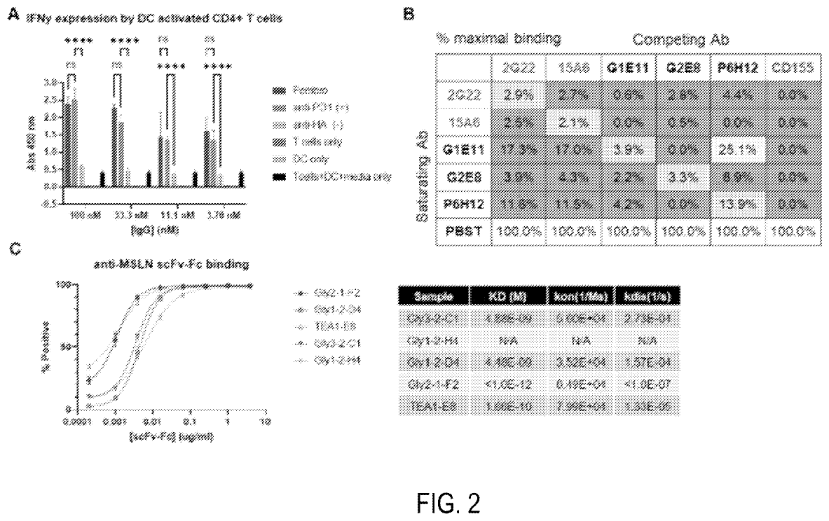

Antibody discovery, engineering, and optimization: We previously built a 27-billion member human phage library that has been used to isolate a number of therapeutic antibodies³³⁻³⁷. Sets of anti-PD1 (FIG. 2 panel A), TIGIT (FIG. 2 panel B), and MSLN (FIG. 2 panel C) have been identified for further engineering and optimization.

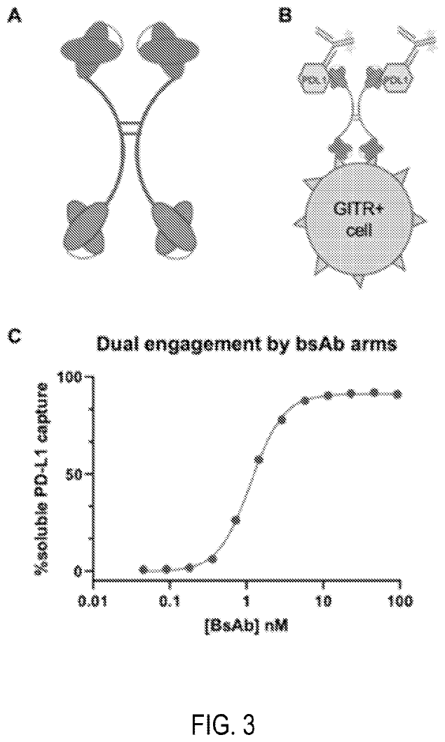

Bi-specific antibody design and engineering: As PD1/TIGIT are expressed on immune cells, immunodepletion is not desired^{38,39}. The current design utilizes a tandem scFv construct previously developed and characterized in the Marasco lab, however other designs will be considered (FIG. 3 panels A-C).

B cell isolation, expansion, and transduction: B cells will be isolated and various expansion media formulations tested^{23,40,41}. For transduction, B cells will be activated, transduced, and sorted 72 hours post transduction.

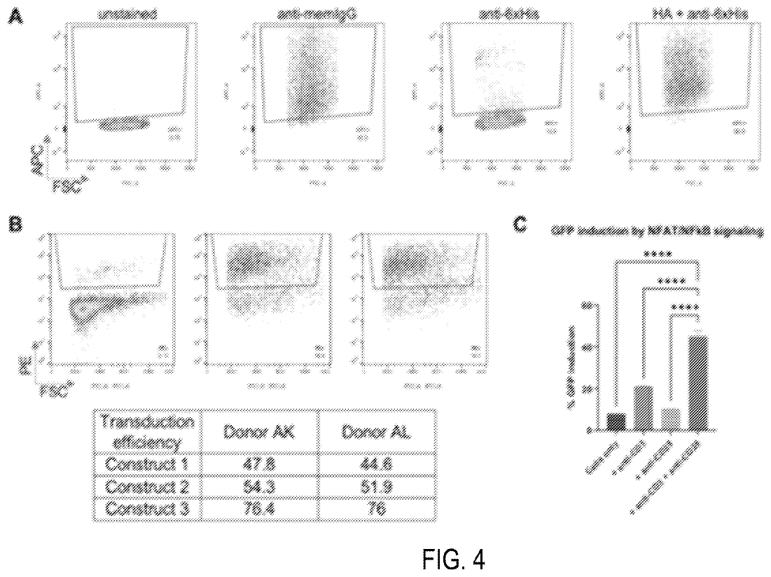

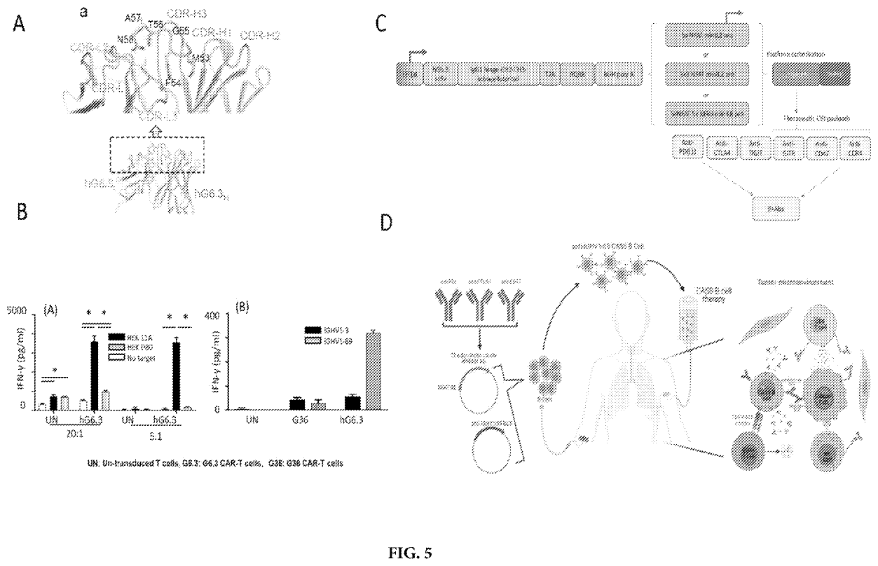

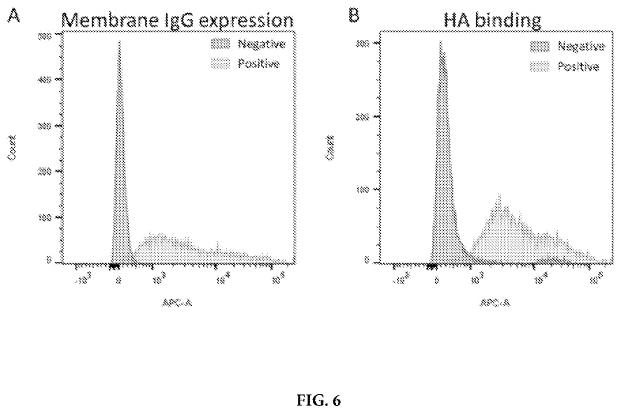

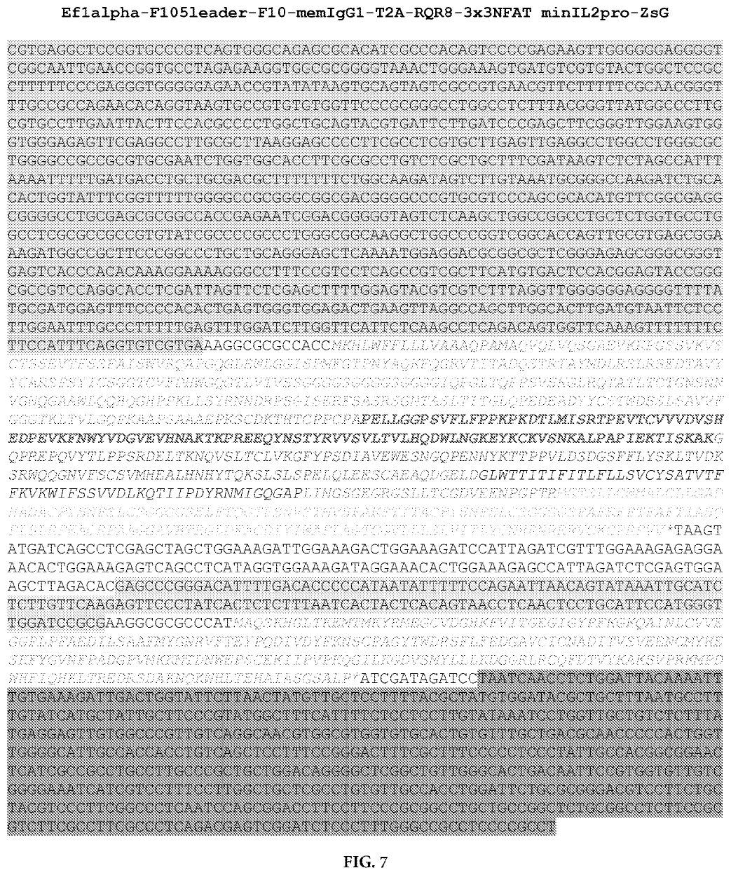

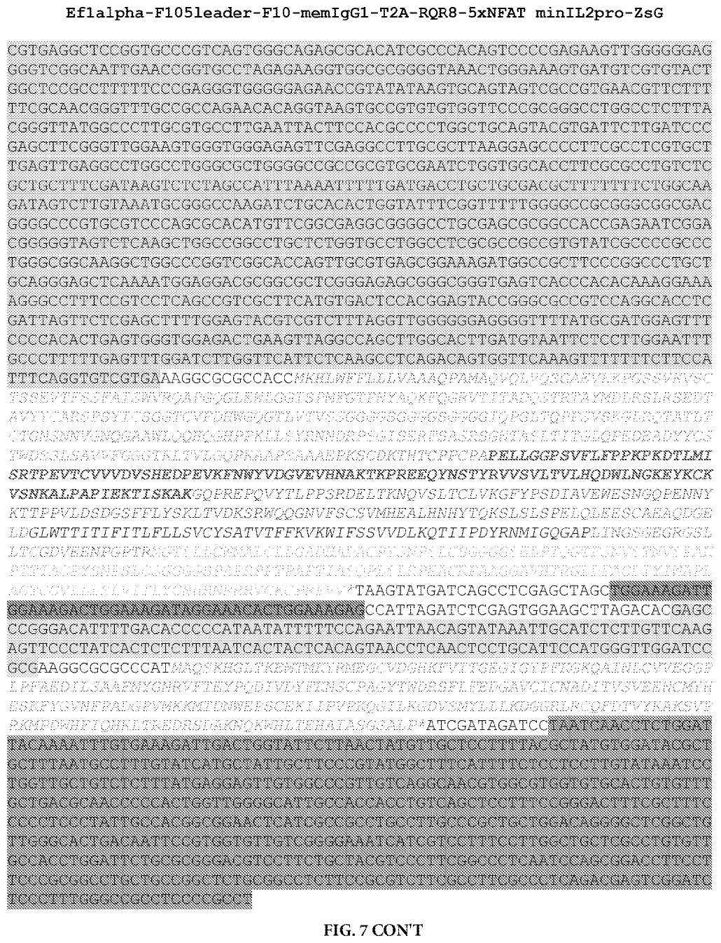

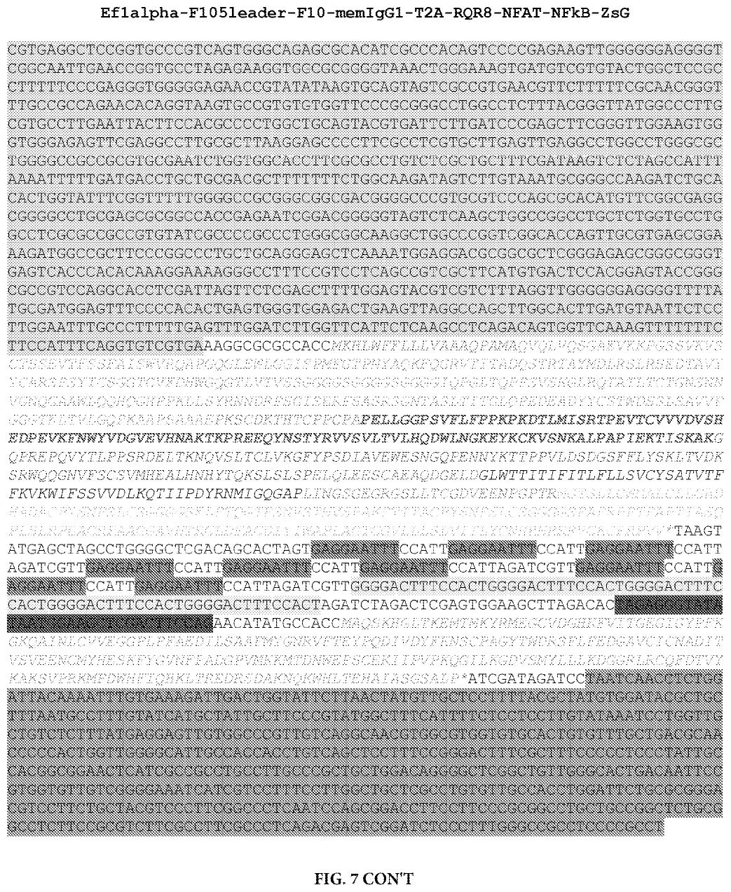

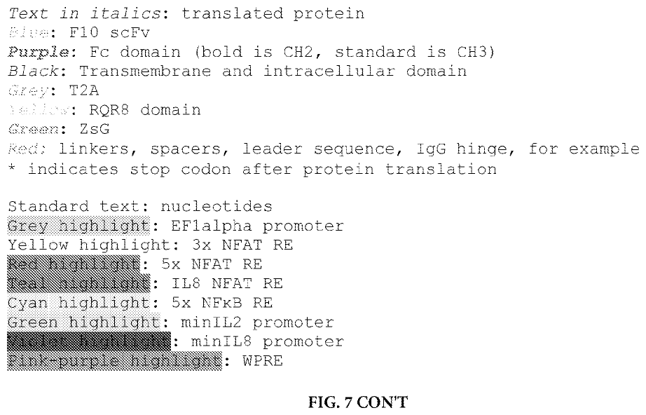

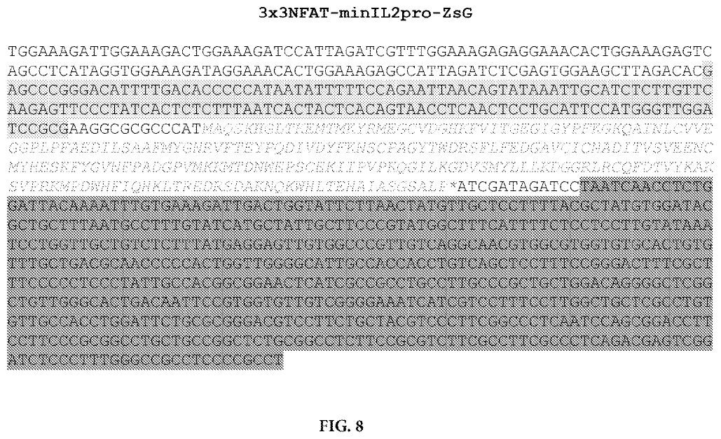

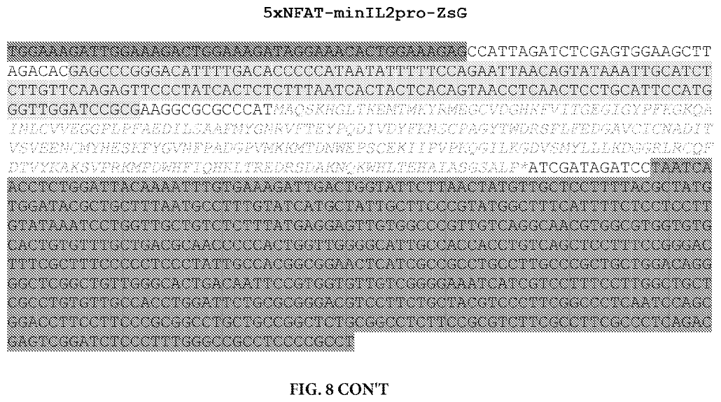

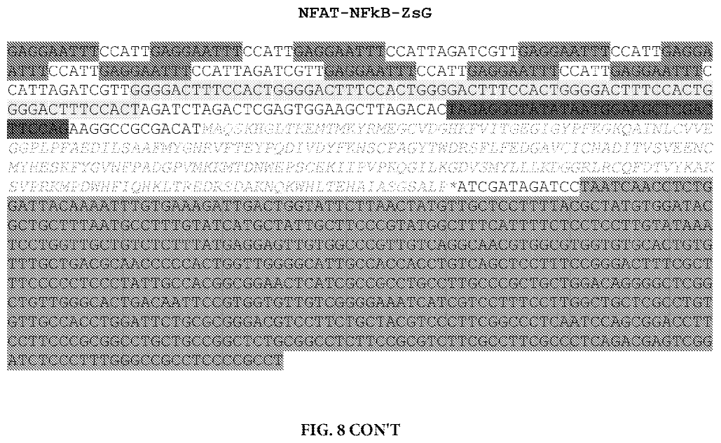

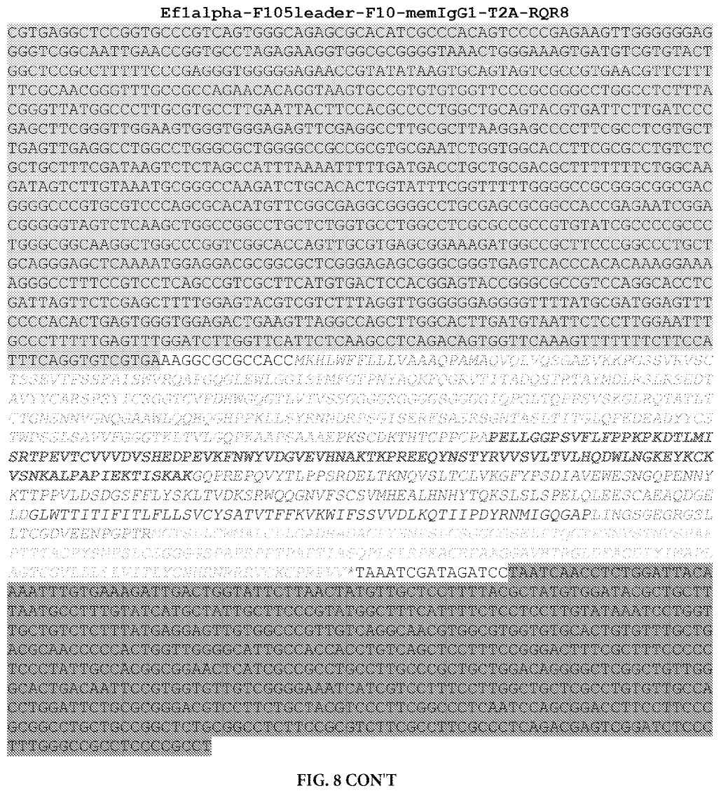

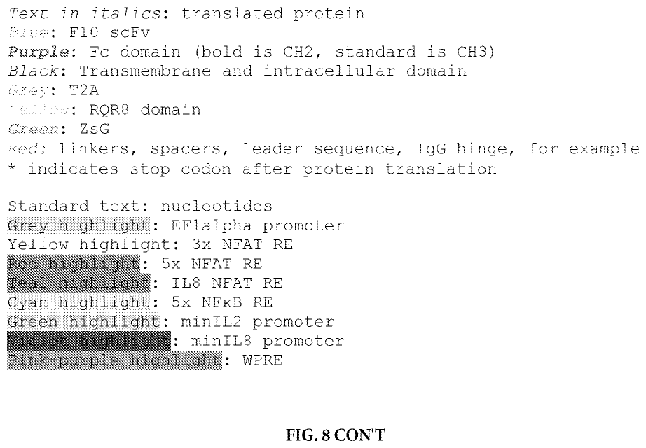

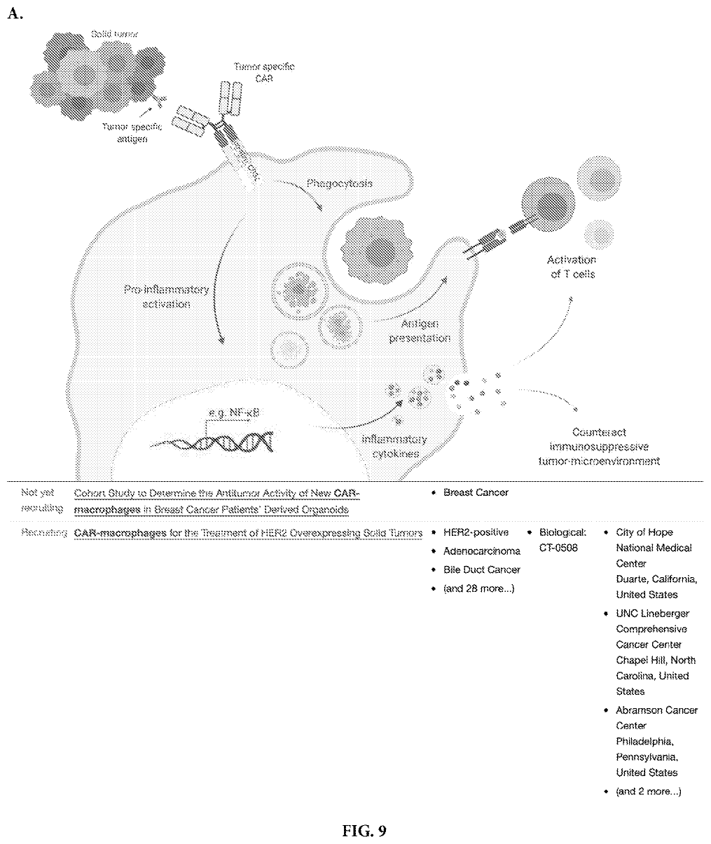

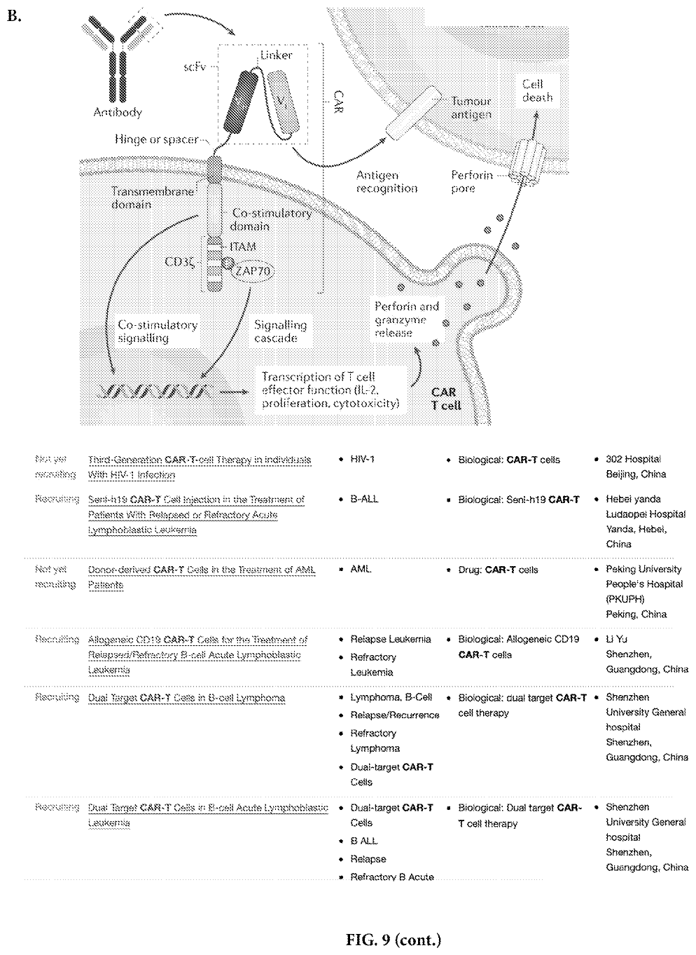

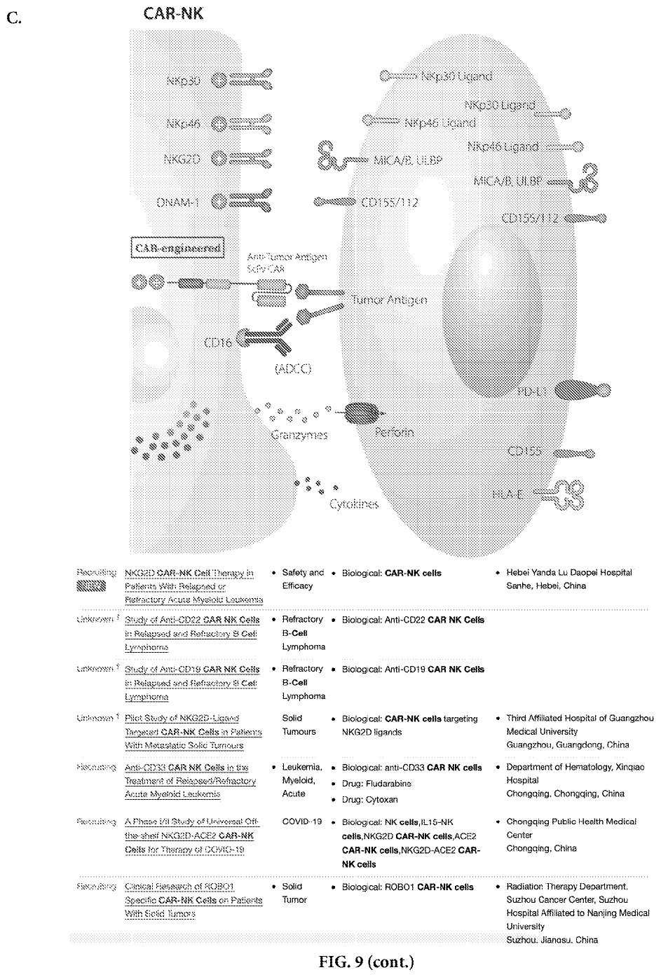

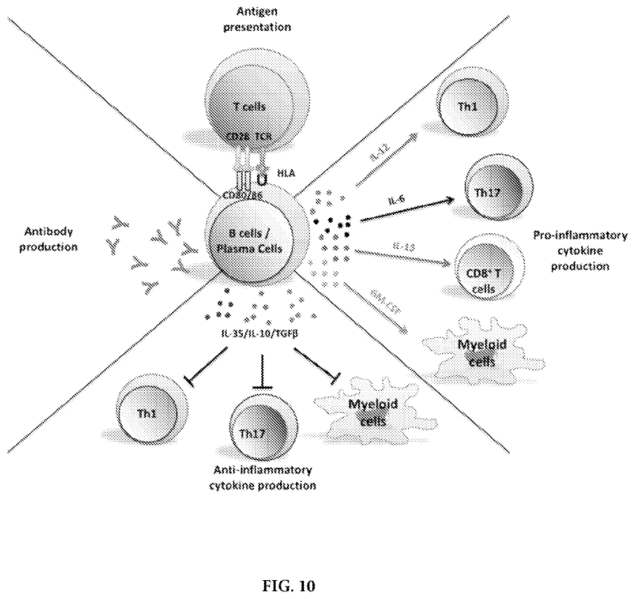

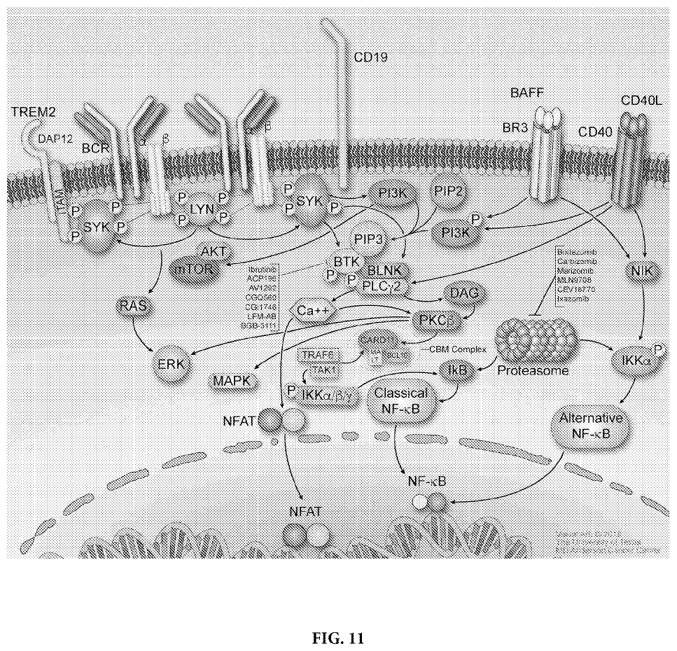

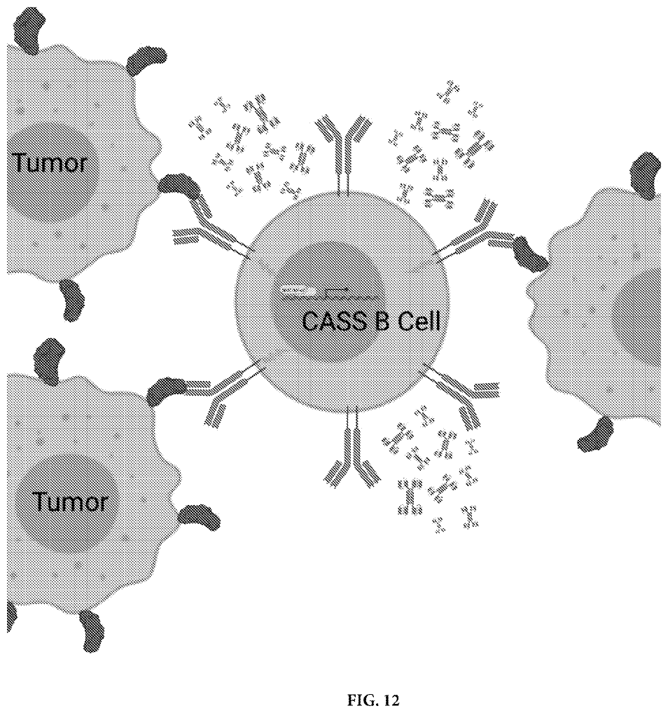

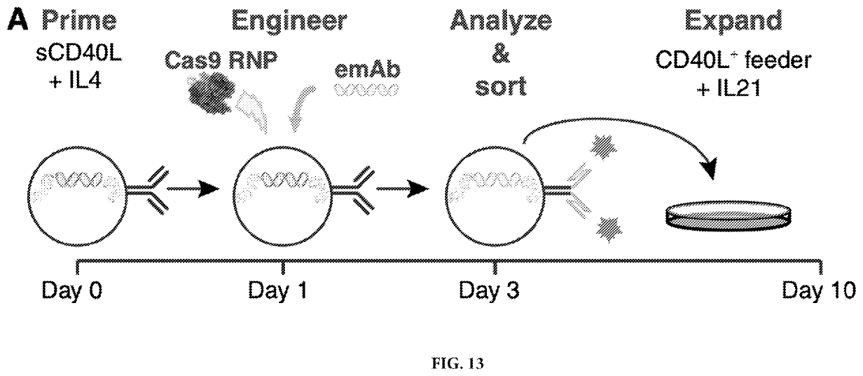

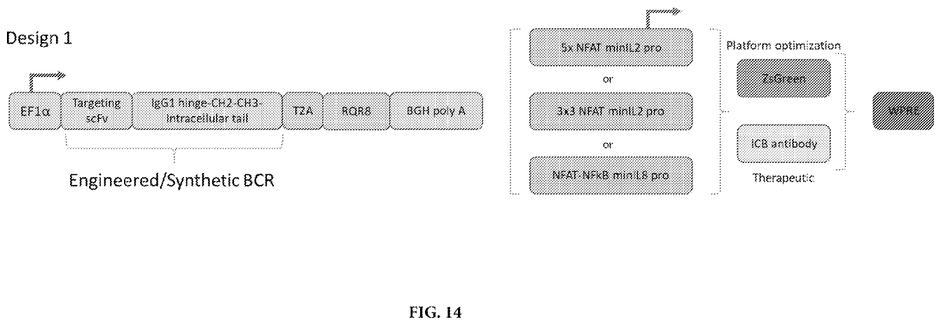

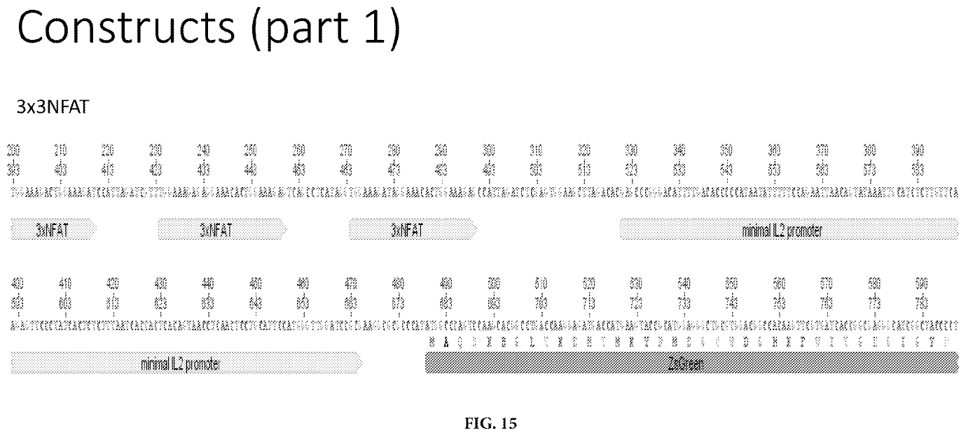

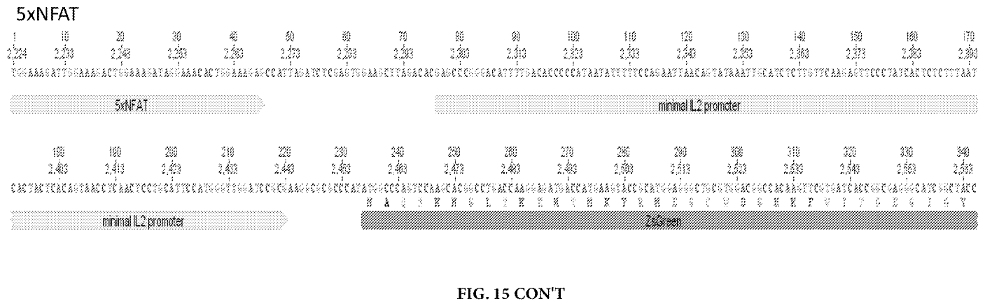

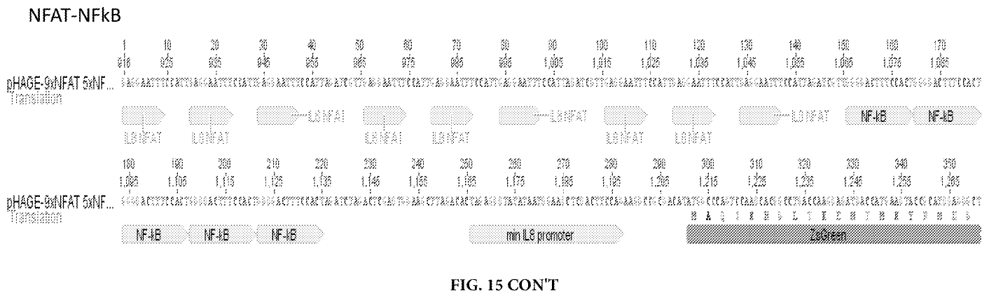

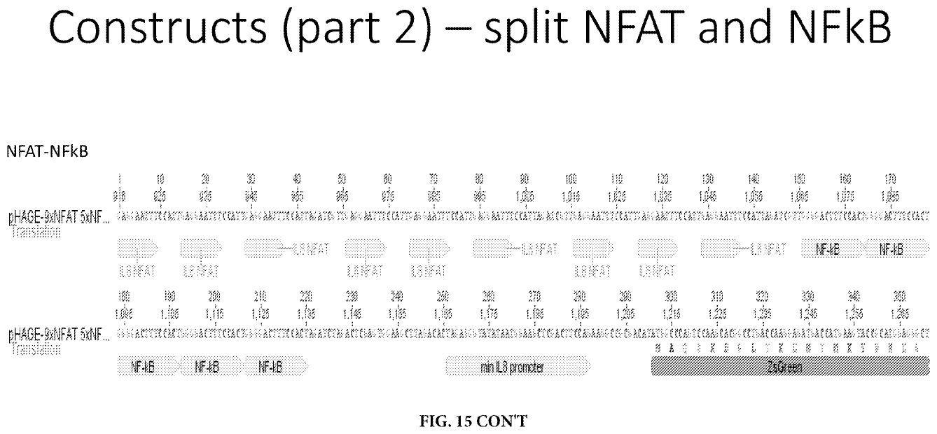

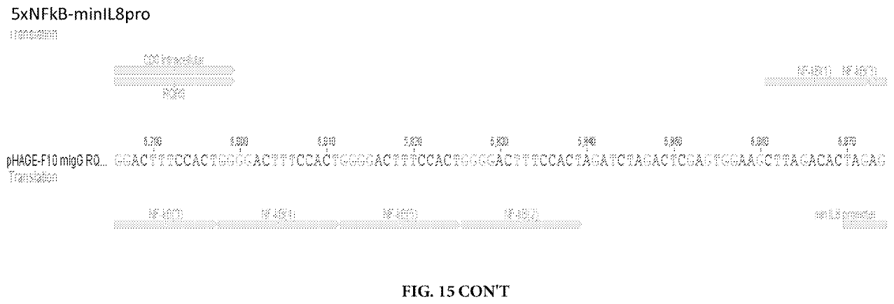

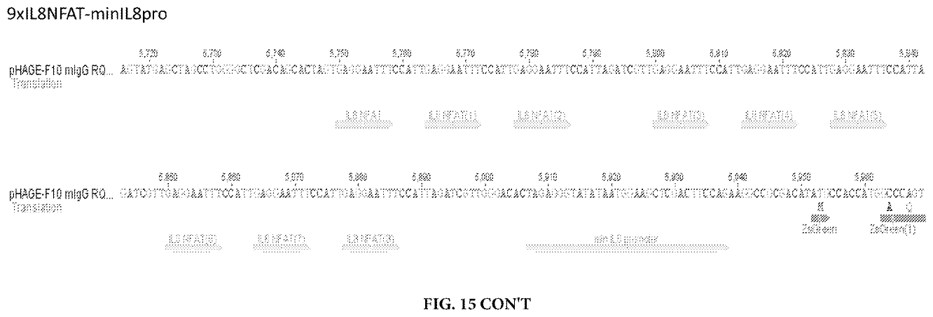

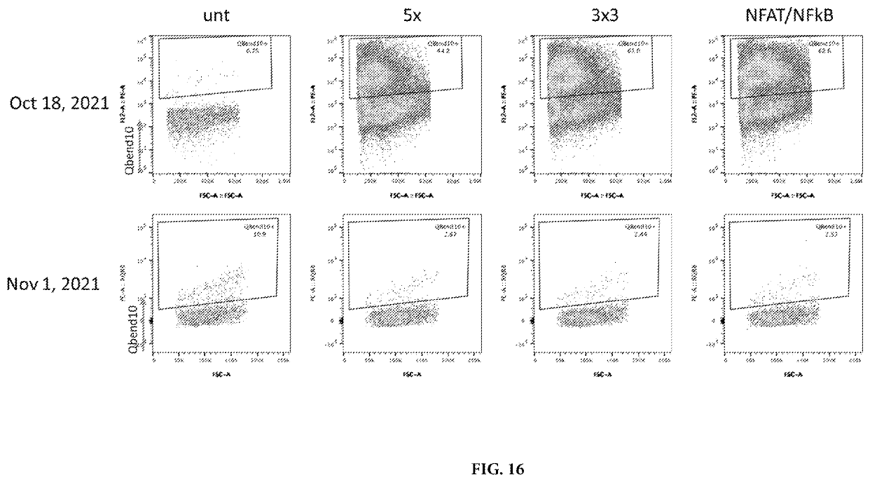

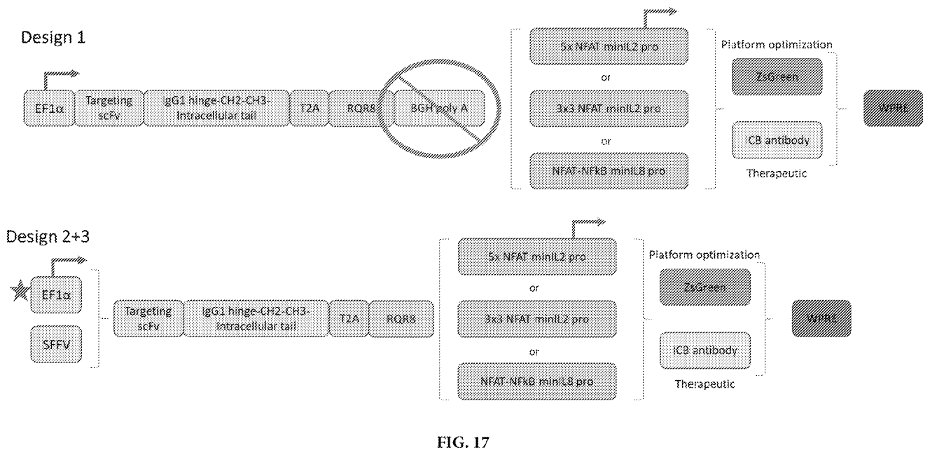

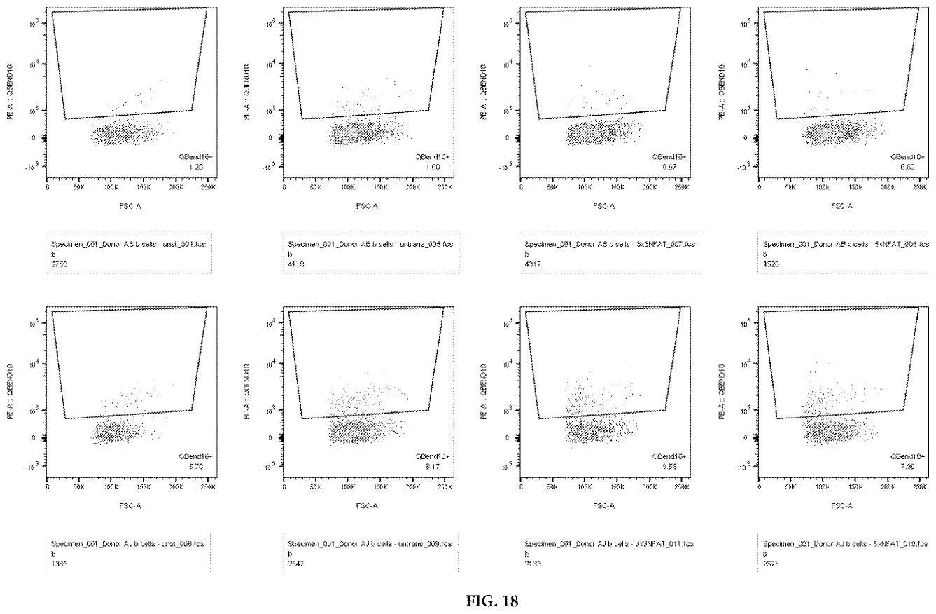

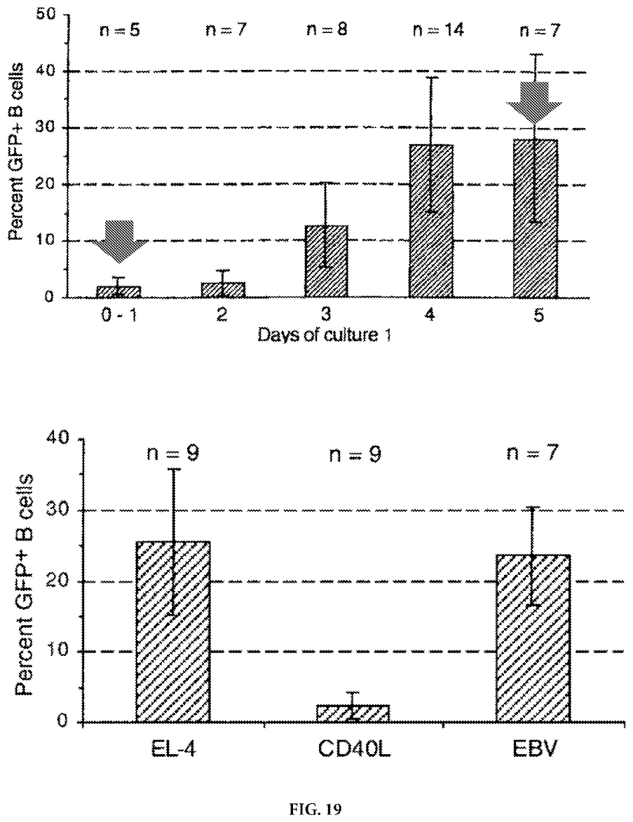

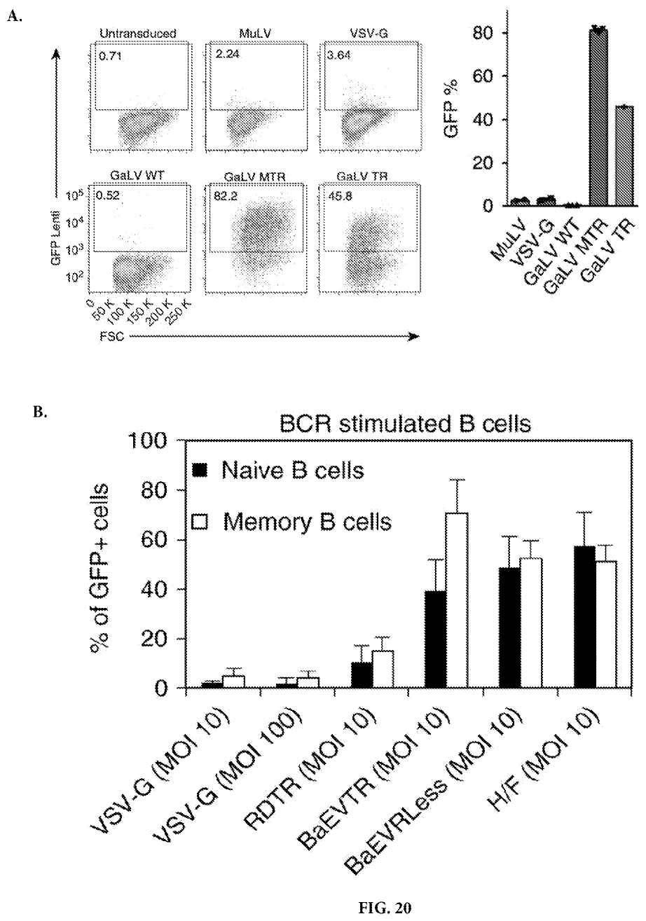

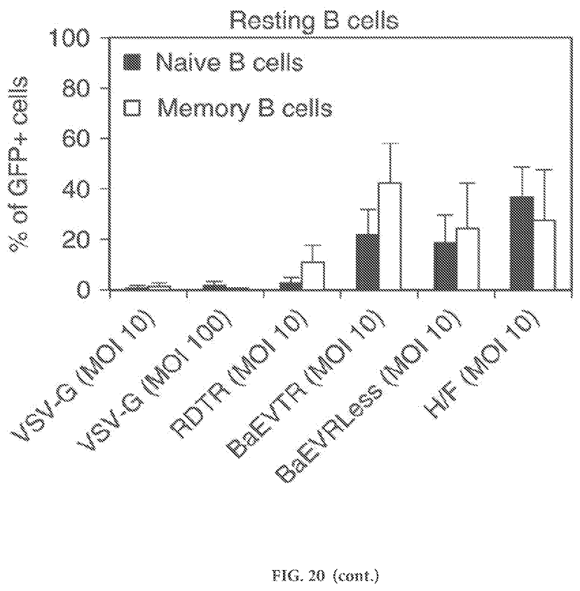

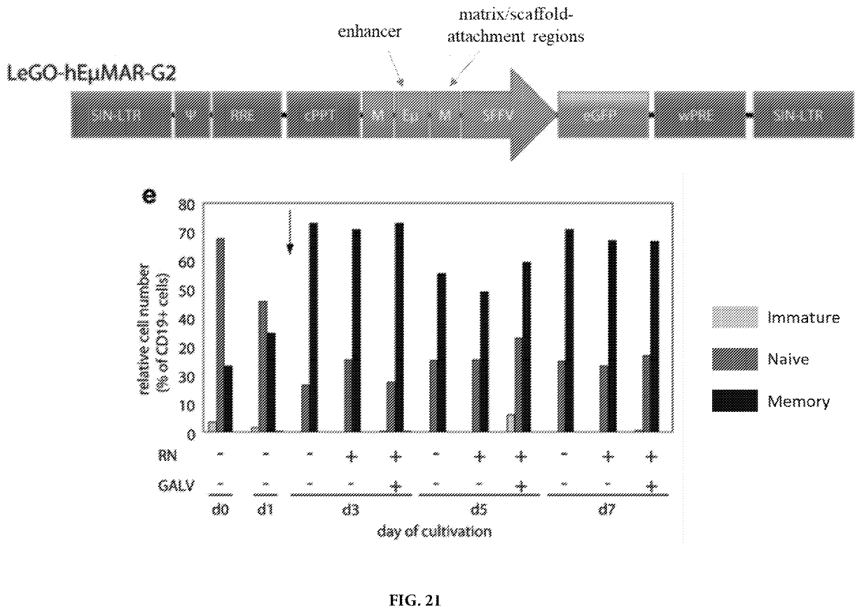

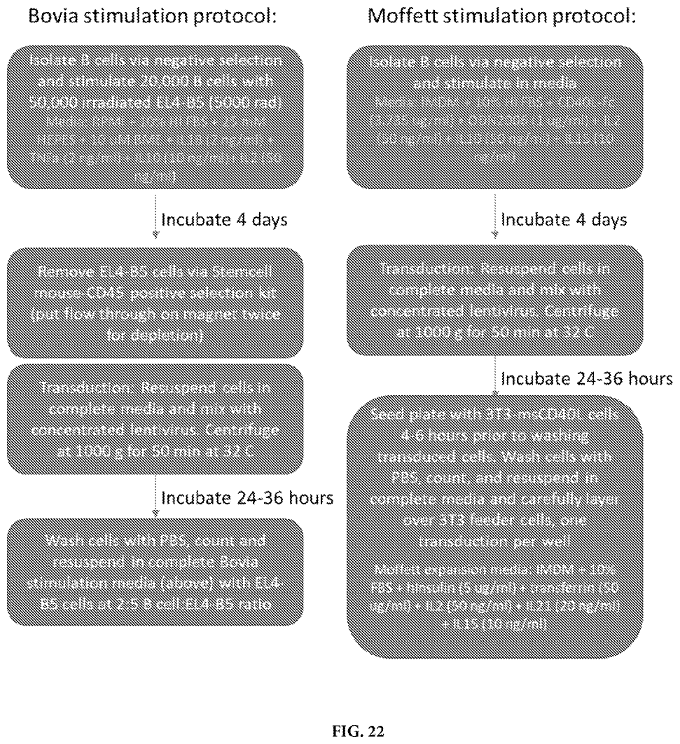

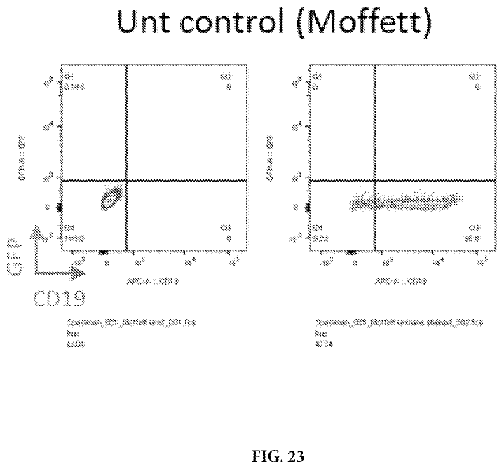

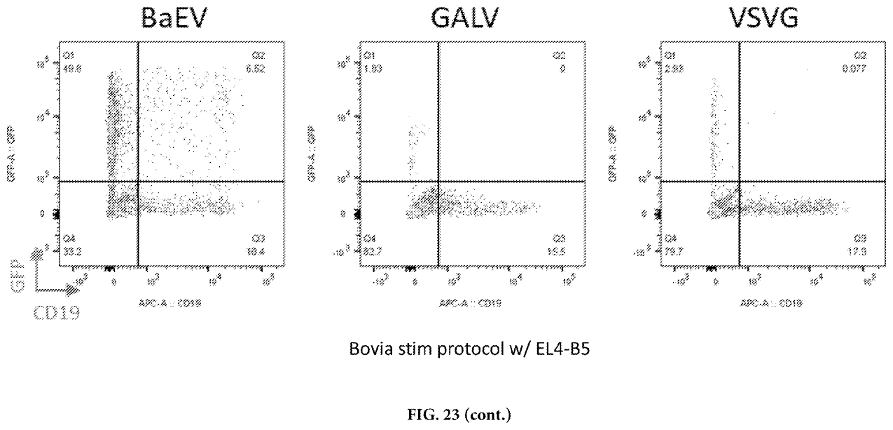

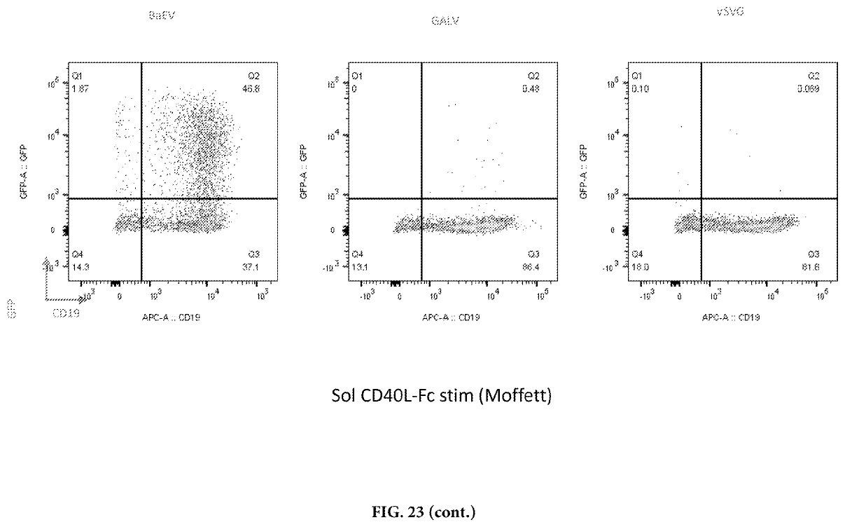

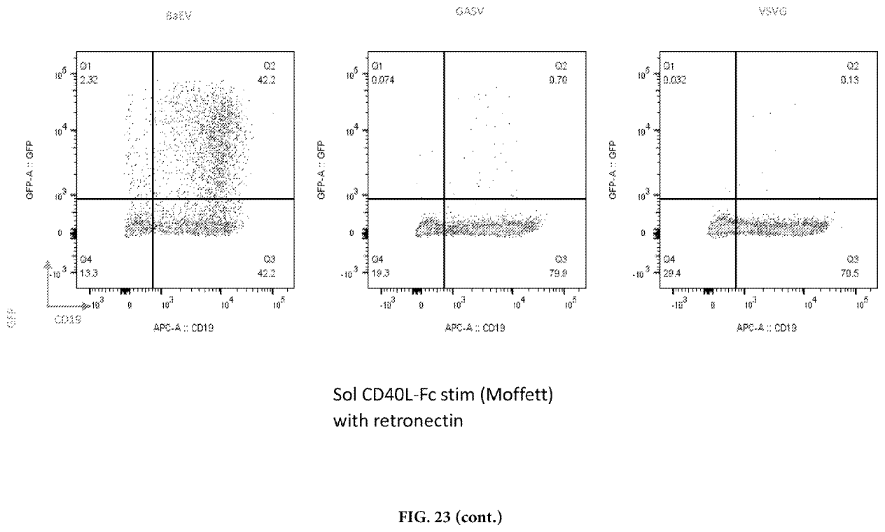

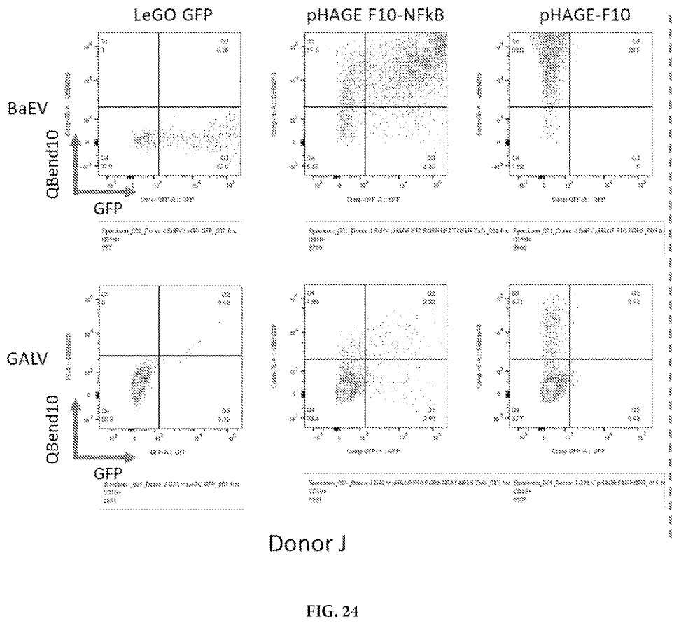

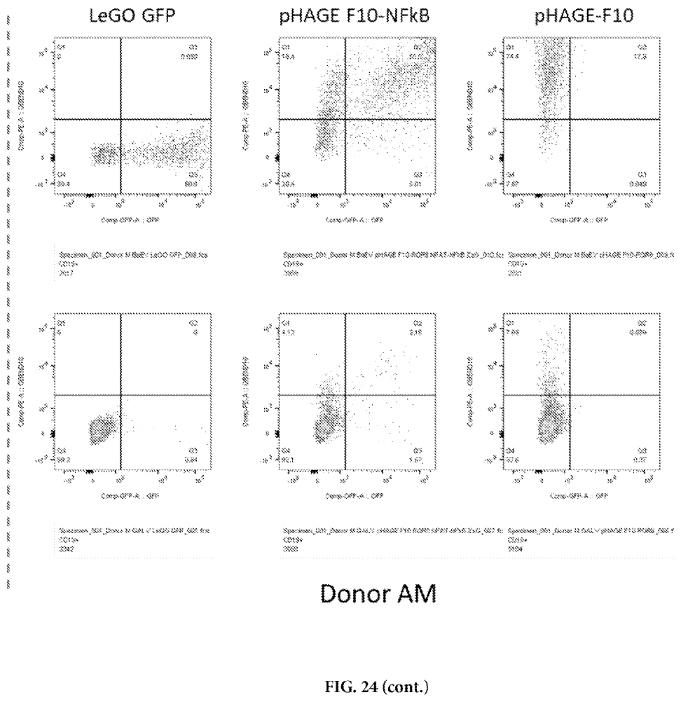

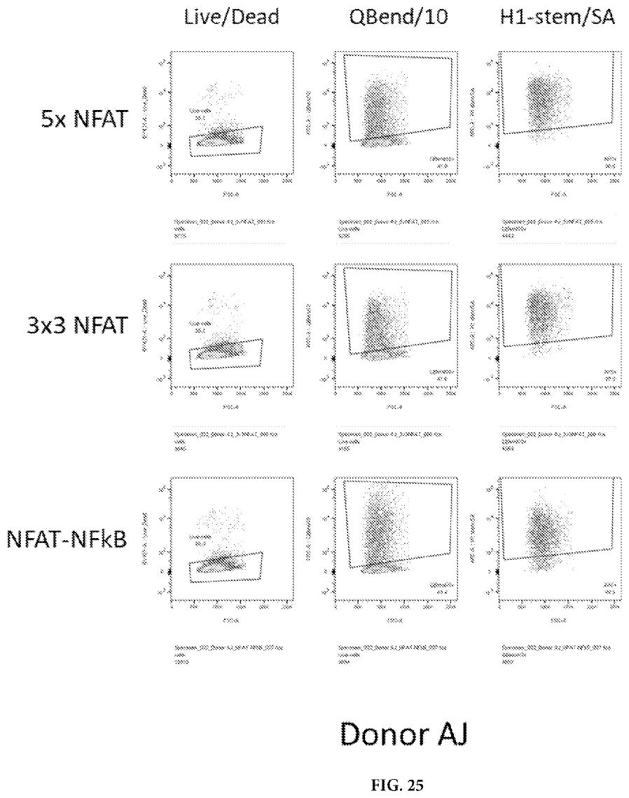

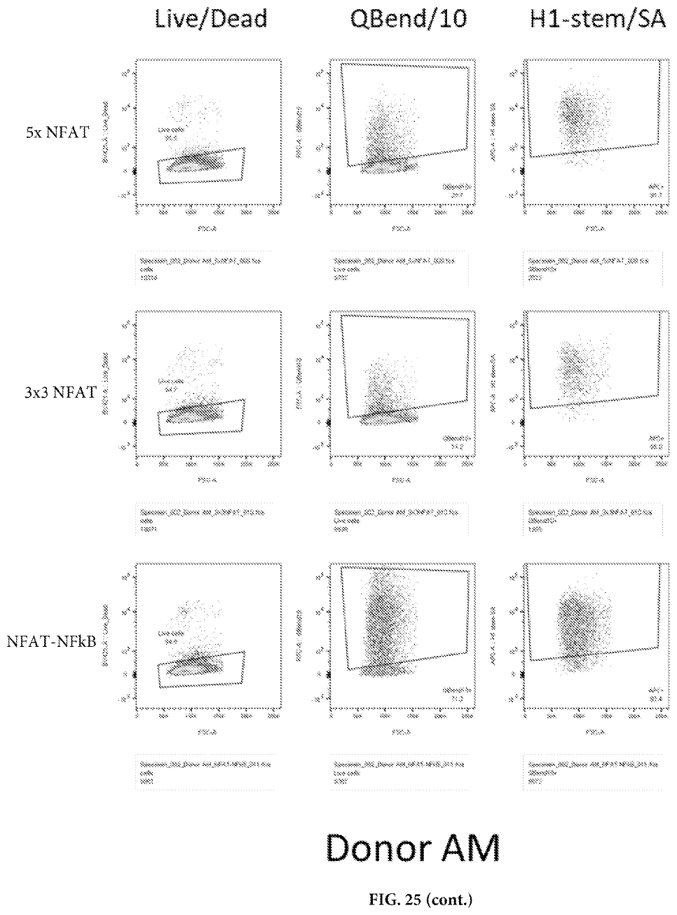

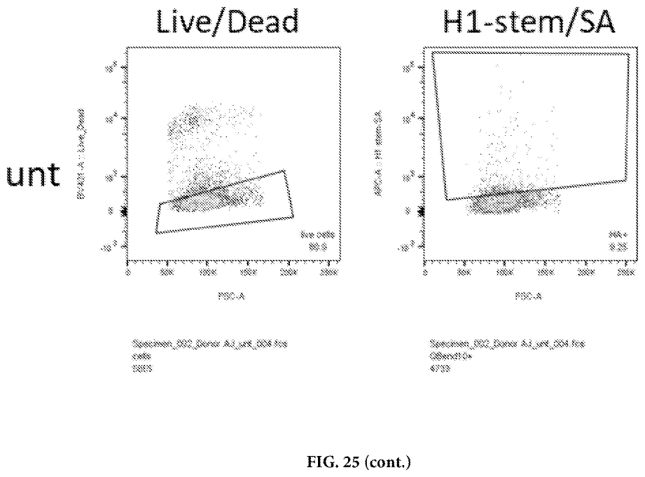

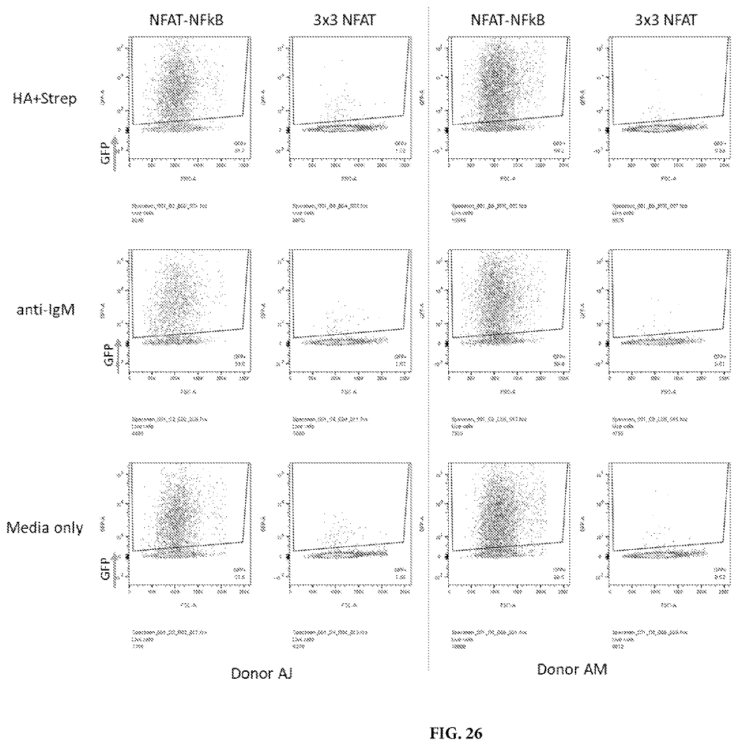

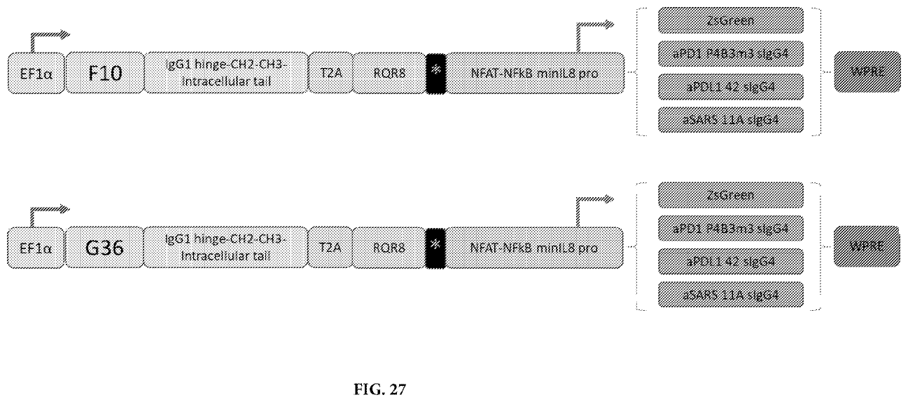

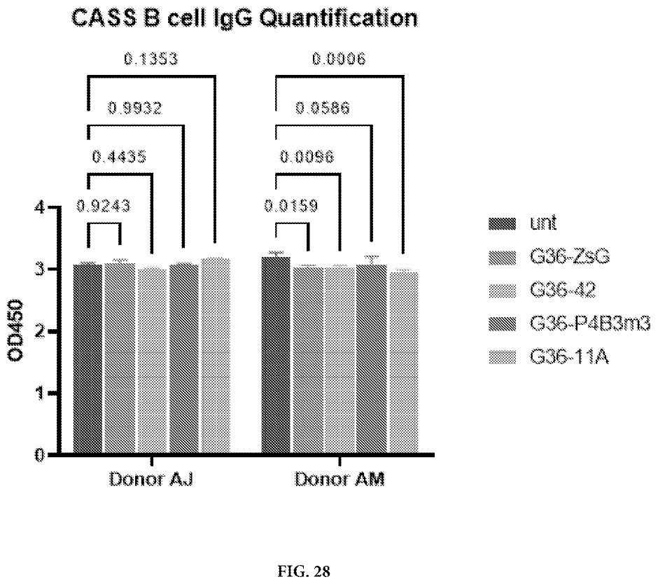

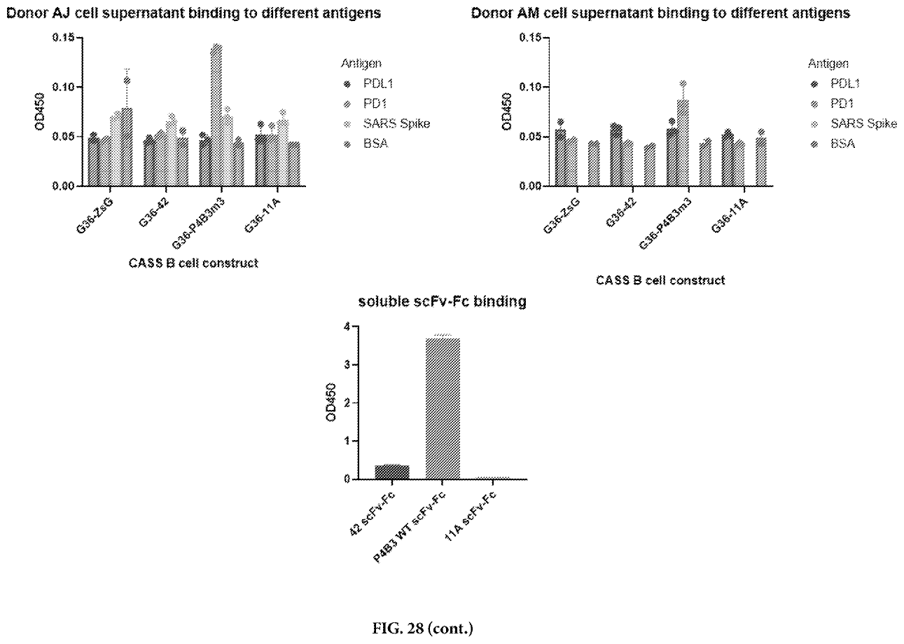

Generation of IgG-BCR construct: Our engineered IgG-BCR is built using an scFv fused to a membrane bound IgG1 hinge-Fc that expresses at high levels and binds the target antigen (FIG. 4 panel A) 42. Transduction experiments using primary B cells demonstrate high titer transduction for multiple donors and DNA constructs (FIG. 4 panel B). We have previously utilized a NFAT/NFkB RE to develop an inducible T cell activation assay (FIG. 4 panel C). For certain experiments, a fluorescent protein will be used in place of the secreted bsAb.

mAb engineering techniques regularly employed by our lab will allow us to develop the panel of mAbs previously discovered to find a lead candidate for each target. Without wishing to be bound by theory, we can achieve high transduction efficiency and exponential expansion of transduced cells for in vivo experiments.

Milestones: The first milestone will be the identification of a lead antibody for each target. The second milestone will be the construction of the vector and successful transduction/expansion of CASS B cells.

Aim 2: In vitro testing and efficacy of CASS B cells-In-depth in vitro characterization will be performed to identify activation thresholds and quantify bsAb secretion levels. Final in vitro assays will be performed using patient-derived organotypic tumor spheroids (PDOTS), allowing us to observe CASS B cell homing and perform detailed analysis of the bsAb payload.

### Experimental Design and Procedures

CASS B cell activation assays: Supernatant from activated CASS B cells will be screened via ELISA to measure bsAb and cytokine concentration.

NSCLC PDOT generation and evaluation: Generation of NSCLC PDOTs will be performed in the lab of Dr. David Barbie following the protocol outlined in Jenkins et al and Aref et al^{43,44}. In addition to testing bsAb and CASS B cell efficacy, a comparative experiment will be performed to identify therapeutic differences between CASS B cell and CAR T cell treatments. Immune profiling via IHC, scRNAseq and cytokine profiling will be performed. Due to the limited availability of NSCLC tumor tissue, a backup plan has been devised to use mesothelioma tumors for PDOT generation. Mesothelioma has a highly suppressive TME and immune infiltrating cells display high levels of exhaustion markers, making this a ideal alternative for in vitro assays⁴⁵⁻⁴⁸.

Without wishing to be bound by theory we can provide an in-depth understanding of CASS B cell activation potential and identify an optimal anti-MSLN scFv for selective targeting of MSLN+tumors. PDOTs will provide a comprehensive data set of CASS B cell efficacy via cytokine secretion and transcriptional profiles, and without wishing to be bound by theory, anti-MSLN CASS B cells will traffic to the tumor and the secreted bsAb will reverse the suppression of immune cells. Additionally, and without wishing to be bound by theory, we can see greater epitope spreading and bystander immune cell activation with the CASS B cell compared to CAR T cell therapy.

Milestones: The first milestone in Aim 2 can be the generation of activation curves for CASS B cells and quantification of bsAb secretion. The next milestone will be establishment of the PDOTS, and efficacy testing of the bsAb systemically and as a CASS B cell payload. The final milestone will be comparing CASS B cell and CAR T cell therapy and the generation of immune profiles for each therapy via IHC and scRNAseq.

Aim 3: In vivo efficacy using HLA matched, humanized NSCLC mouse models-In vivo experiments will be performed on a cell line derived xenograft (CDX) model in humanized mice. The use of a validated patient derived xenograft (PDX) model from a publicly available repository for final testing will improve tumor integrity and phenotypic characteristics while accurately mimicking the TME of in vivo tumors.

### Experimental Design and Procedures

Establishment of an NSCLC CDX and PDX model in humanized mice: NSCLC cell lines and PDX models will be screened for PDL1 and CD155 expression levels prior to luciferization⁴⁹. Human immune system reconstitution will be performed as reported by ourselves and others⁵⁰⁻⁵². To generate growth curves, varying concentrations of NSCLC cell lines/PDX tumors will be implanted into the rear flank of a humanized mouse⁵³.

Assessment of CASS B cell efficacy in humanized NSCLC mouse model: We will use the CDX and PDX models to test the efficacy of the bsAb and CASS B cell in vivo. Samples will be analyzed by multiparameter FACS, IHC/ISH, and scRNASeq following established protocols regularly used in our lab⁵⁴⁻⁶³.

We have significant experience with generating humanized mice and CDX/PDX models, and will successfully develop a NSCLC model. These models will demonstrate that CASS B cells cluster around the tumor and secrete high levels of bsAb, decreasing the immunosuppressive nature of the TME and recruiting additional anti-tumor immune cells. IHC/ISH and scRNAseq will provide molecular evidence of bsAb efficacy, CASS B cell homing, and activation of APC pathways in both CASS B cells and CD4 T cells, while 5′ scRNAseq of the TCR/BCRs will be used to directly monitor epitope spreading among TILs.

Statistical considerations: With 5 animals per group for individual pair-wise comparisons between conditions of interest and different outcome measures we have power of 0.93 to detect a difference in the means equal to 2.5SD using two-sample two-tailed t-test at 0.05 level. Additional statistical analysis will be provided by consultation with the Dana-Farber Biostatistics Core.

Milestones: The first milestone in Aim 3 will be the generation of CDX and PDX models for NSCLC. Subsequent milestones would be initiation and completion of the planned animal experiments testing the bsAb in a CDX model, and the CASS B cell in CDX and PDX models. Due to the wealth of data generated by these experiments, tertiary milestones would be completion of data analysis for each animal experiment.

### REFERENCES CITED IN THIS EXAMPLE

1. Ho, M. et al. Mesothelin expression in human lung cancer. Clin. Cancer Res. 13, 1571-1575 (2007).
2. Thomas, A. et al. High mesothelin expression in advanced lung adenocarcinoma is associated with KRAS mutations and a poor prognosis. Oncotarget 6, 11694-11703 (2015).
3. Weidemann, S. et al. Mesothelin Expression in Human Tumors: A Tissue Microarray Study on 12,679 Tumors. Biomedicines 9, 4-9 (2021).
4. Berghmans, T., Durieux, V., Hendriks, L. E. L. & Dingemans, A.-M. Immunotherapy: From Advanced NSCLC to Early Stages, an Evolving Concept. Front. Med. 7, 1-16 (2020).
5. Qu, J. et al. The progress and challenge of anti-PD-1/PD-L1 immunotherapy in treating non-small cell lung cancer. Ther. Adv. Med. Oncol. 13, 1-28 (2021).
6. Ott, P. A. & Wu, C. J. Cancer vaccines: Steering t cells down the right path to eradicate tumors. Cancer Discov. 9, 476-481 (2019).
7 Rogado, J. et al. Immune-related adverse events predict the therapeutic efficacy of anti-PD-1 antibodies in cancer patients. Eur. J. Cancer 109, 21-27 (2019).
8. Lee, D. H. Update of early phase clinical trials in cancer immunotherapy. BMB Rep. 54, 70-88 (2021).
9. Ahn, M.-J. et al. 1400P Vibostolimab, an anti-TIGIT antibody, as monotherapy and in combination with pembrolizumab in anti-PD-1/PD-L1-refractory NSCLC. Ann. Oncol. 31, S887 (2020).
10. Niu, J. et al. 1410P Safety and efficacy of vibostolimab, an anti-TIGIT antibody, plus pembrolizumab in patients with anti-PD-1/PD-L1-naive NSCLC. Ann. Oncol. 31, S891-S892 (2020).
11. Rodriguez-Abreu, D. et al. Primary analysis of a randomized, double-blind, phase II study of the anti-TIGIT antibody tiragolumab (tira) plus atezolizumab (atezo) versus placebo plus atezo as first-line (1L) treatment in patients with PD-L1-selected NSCLC (CITYSCAPE). J. Clin. Oncol. 38, 9503 (2020).
12. Suarez, E. R. et al. Chimeric antigen receptor T cells secreting anti-PD-L1 antibodies more effectively regress renal cell carcinoma in a humanized mouse model. Oncotarget 7, (2016).
13. Hawkins, E. R., D'souza, R. R. & Klampatsa, A. Armored CAR T-cells: The next chapter in T-cell cancer immunotherapy. Biol. Targets Ther. 15, 95-105 (2021).
14. Mukhopadhyay, M. Macrophages enter CAR immunotherapy. Nat. Methods 17, 561 (2020).
15. Oelsner, S. et al. Continuously expanding CAR NK-92 cells display selective cytotoxicity against B-cell leukemia and lymphoma. Cytotherapy 19, 235-249 (2017).
16. Kreiter, S. et al. Mutant MHC class II epitopes drive therapeutic immune responses to cancer. Nature 520, 692-696 (2015).
17. Alspach, E. et al. MHC-II neoantigens shape tumour immunity and response to immunotherapy. Nature 574, 696-701 (2019).
18. Chmielewski, M., Kopecky, C., Hombach, A. A. & Abken, H. IL-12 release by engineered T cells expressing chimeric antigen receptors can effectively muster an antigen-independent macrophage response on tumor cells that have shut down tumor antigen expression. Cancer Res. 71, 5697-5706 (2011).
19. Rafiq, S. et al. Targeted delivery of a PD-1-blocking scFV by CAR-T cells enhances anti-tumor efficacy in vivo. Nat. Biotechnol. 36, 847-858 (2018).
20. Iwahori, K. et al. Engager T cells: A new class of antigen-specific t cells that redirect bystander T cells. Mol. Ther. 23, 171-178 (2015).
21. Voss, J. E. et al. Reprogramming the antigen specificity of b cells using genome-editing technologies. Elife 8, 1-22 (2019).
22. Nahmad, A. D. et al. Engineered B cells expressing an anti-HIV antibody enable memory retention, isotype switching and clonal expansion. Nat. Commun. 11, 1-10 (2020).
23. Moffett, H. F. et al. B cells engineered to express pathogen-specific antibodies protect against infection. Sci. Immunol. 4, (2019).
24. Nahmad, A. D. et al. Engineered B cells expressing an anti-HIV antibody enable memory retention, isotype switching and clonal expansion. Nat. Commun. 11, 1-10 (2020).
25. Uchibori, R. et al. Functional Analysis of an Inducible Promoter Driven by Activation Signals from a Chimeric Antigen Receptor. Mol. Ther.—Oncolytics 12, 16-25 (2019).
26. Zhang, L. et al. Improving adoptive T cell therapy by targeting and controlling IL-12 expression to the tumor environment. Mol. Ther. 19, 751-759 (2011).
27. Hooijberg, E., Bakker, A. Q., Ruizendaal, J. J. & Spits, H. NFAT-controlled expression of GFP permits visualization and isolation of antigen-stimulated primary human T cells. Blood 96, 459-466 (2000).
28. Lin, T. et al. Establishment of NF-κB sensing and interleukin-4 secreting mesenchymal stromal cells as an “on-demand” drug delivery system to modulate inflammation. Cytotherapy 19, 1025-1034 (2017).
29. Girard-Gagnepain, A. et al. Baboon envelope pseudotyped LVs outperform VSV-G-LVs for gene transfer into early-cytokine-stimulated and resting HSCs. Blood 124, 1221-1231 (2014).
30. Tomás, H. A. et al. Improved GaLV-TR Glycoproteins to Pseudotype Lentiviral Vectors: Impact of Viral Protease Activity in the Production of LV Pseudotypes. Mol. Ther.—Methods Clin. Dev. 15, 1-8 (2019).
31. Bernadin, O. et al. Baboon envelope LVs efficiently transduced human adult, fetal, and progenitor T cells and corrected SCID-X1 T-cell deficiency. Blood Adv. 3, 461-475 (2019).
32. Winiarska, M. et al. Selection of an optimal promoter for gene transfer in normal B cells. Mol. Med. Rep. 16, 3041-3048 (2017).
33. Xu, C. et al. Unique biological properties of catalytic domain directed human anti-CAIX antibodies discovered through phage-display technology. PLOS One 5, (2010).
34. Sui, J. et al. Structural and functional bases for broad-spectrum neutralization of avian and human influenza A viruses. Nat. Struct. Mol. Biol. 16, 265-273 (2009).
35. Fu, Y. et al. A broadly neutralizing anti-influenza antibody reveals ongoing capacity of haemagglutinin-specific memory B cells to evolve. Nat. Commun. 7, (2016).
36. Chang, D.-K. et al. Humanization of an anti-CCR4 antibody that kills cutaneous T-cell lymphoma cells and abrogates suppression by T-regulatory cells. Mol. Cancer Ther. 11, 2451-2461 (2012).
37. Chang, D. K. et al. Humanized mouse G6 anti-idiotypic monoclonal antibody has therapeutic potential against IGHV1-69 germline gene-based B-CLL. MAbs 8, 787-798 (2016).
38. Marin-Acevedo, J. A. et al. Next generation of immune checkpoint therapy in cancer: New developments and challenges. J. Hematol. Oncol. 11, 1-20 (2018).
39. Saunders, K. O. Conceptual approaches to modulating antibody effector functions and circulation half-life. Front. Immunol. 10, 1-20 (2019).
40. Su, K.-Y., Watanabe, A., Yeh, C.-H., Kelsoe, G. & Kuraoka, M. Efficient Culture of Human Naive and Memory B Cells for Use as APCs. J. Immunol. 197, 4163-4176 (2016).
41. Kuraoka, M. et al. Complex Antigens Drive Permissive Clonal Selection in Germinal Centers. Immunity 44, 542-552 (2016).
42. Wan, Z. et al. The activation of IgM- or isotype-switched IgG- and IgE-BCR exhibits distinct mechanical force sensitivity and threshold. Elife 4, 1-24 (2015).
43. Aref, A. R. et al. 3D microfluidic: Ex vivo culture of organotypic tumor spheroids to model immune checkpoint blockade. Lab Chip 18, 3129-3143 (2018).
44. Jenkins, R. W. et al. Ex Vivo Profiling of PD-1 Blockade Using Organotypic Tumor Spheroids. Cancer Discov. 176, 139-148 (2018).
45. Lv, J. & Li, P. Mesothelin as a biomarker for targeted therapy. Biomark. Res. 7, (2019).
46. Marcq, E. et al. Abundant expression of TIM-3, LAG-3, PD-1 and PD-L1 as immunotherapy checkpoint targets in effusions of mesothelioma patients. Oncotarget 8, 89722-89735 (2017).
47. Klampatsa, A. et al. Phenotypic and functional analysis of malignant mesothelioma tumor-infiltrating lymphocytes. Oncoimmunology 8, 1-12 (2019).
48. Alay, A. et al. Integrative transcriptome analysis of malignant pleural mesothelioma reveals a clinically relevant immune-based classification. J. Immunother. Cancer 9, (2021).
49. Liu, J. F. et al. Establishment of patient-derived tumor xenograft models of epithelial ovarian cancer for preclinical evaluation of novel therapeutics. Clin. Cancer Res. 23, 1263-1273 (2017).
50. Biswas, S. et al. Humoral immune responses in humanized BLT mice immunized with West Nile virus and HIV-1 envelope proteins are largely mediated via human CD5+ B cells. Immunology 134, 419-433 (2011).
51. Chang, H. et al. Human B-cell ontogeny in humanized NOD/SCID γc(null) mice generates a diverse yet auto/poly- and HIV-1-reactive antibody repertoire. Genes Immun. 13, 399-410 (2012).
52. Billerbeck, E. et al. Development of human CD4+FoxP3+ regulatory T cells in human stem cell factor-, granulocyte-macrophage colony-stimulating factor-, and interleukin-3-expressing NOD-SCID IL2Rγnull humanized mice. Blood 117, 3076-3086 (2011).
53. Lin, S. et al. Establishment of peripheral blood mononuclear cell-derived humanized lung cancer mouse models for studying efficacy of PD-L1/PD-1 targeted immunotherapy. MAbs 10, 1301-1311 (2018).
54. Stoeckius, M. et al. Cell Hashing with barcoded antibodies enables multiplexing and doublet detection for single cell genomics. Genome Biol. 19, 1-12 (2018).
55. Tian, L. et al. scPipe: A flexible R/Bioconductor preprocessing pipeline for single-cell RNA-sequencing data. PLOS Comput. Biol. 14, 1-15 (2018).
56. Amezquita, R. A. et al. Orchestrating single-cell analysis with Bioconductor. Nat. Methods 17, 137-145 (2020).
57. Hao, Y. et al. Integrated analysis of multimodal single-cell data. Cell 184, 3573-3587.e29 (2021).
58. Lytal, N., Ran, D. & An, L. Normalization Methods on Single-Cell RNA-seq Data: An Empirical Survey. Front. Genet. 11, 1-14 (2020).
59. McCarthy, D. J., Campbell, K. R., Lun, A. T. L. & Wills, Q. F. Scater: Pre-processing, quality control, normalization and visualization of single-cell RNA-seq data in R. Bioinformatics 33, 1179-1186 (2017).
60. Haghverdi, L., Lun, A. T. L., Morgan, M. D. & Marioni, J. C. Batch effects in single-cell RNA-sequencing data are corrected by matching mutual nearest neighbors. Nat. Biotechnol. 36, 421-427 (2018).
61. Chen, Y. et al. edgeR: differential analysis of sequence read count data User's Guide. R Packag. 1-121 (2020).
62. Subramanian, A. et al. Gene set enrichment analysis: A knowledge-based approach for interpreting genome-wide expression profiles. Proc. Natl. Acad. Sci. U.S.A. 102, 15545-15550 (2005).
63. DeTomaso, D. et al. Functional interpretation of single cell similarity maps. Nat. Commun. 10, (2019).

### Example 3

### Use of CASS B Cells and Patient Derived Tumor Cells In Vitro and In Vivo to Explore the B-CLL TME

Chimeric Antibody Signaling and Secretion (CASS) B cells will be used to study B CLL tumor microenvironment (TME) ex vivo and in tumor bearing mice. CASS B cells inducibly secrete checkpoint blockade modulator Abs (e.g., checkpoint inhibitor Abs) at the tumor site. Unlike CAR T and NK cells, CASS B cells are antigen presenting cells (APCs) and upon reversal of T cell exhaustion will promote neoantigen recognition and spreading. Studies on tumor spheroids and in tumor bearing mice will be pursued.

Over the past decade, immune checkpoint blockade inhibitor (CBI) antibodies and CAR T cells have revolutionized the way we treat cancer. While both of these therapies engage the patient's immune system, neither is able to proactively initiate an anti-tumor immune response and significant limitations exist in their scope of use and efficacy. To address this, we will develop Chimeric Antibody Secreting and Signaling (CASS) B cells, which express an engineered, tumor targeting B cell receptor (BCR) and upon engagement, will secrete high levels of CBIs locally at the tumor site. As B cells also serve as professional antigen presenting cells, they can process and present antigens on MHC class II molecules, further enhancing immune cell recognition of the tumor and assisting in neoantigen spreading. A key component of immunologic memory, CASS B cells will simultaneously recruit a wide range of immune cells and reverse tumor infiltrating lymphocyte exhaustion, providing a robust and lifelong surveillance program protecting against tumor metastasis and recurrence.

B CLL was chosen as a model to develop an anti-IGHV1-69 directed CASS B cell secreting an immunomodulatory CBIs. The finding that IGHV1-69 encoded BCRs expressed on B-CLL cells are always unmutated VH segments provides an opportunity for anti-idiotype CASS B cell therapy. We have humanized G6 (hG6.3) mAb, completed co-crystallographic studies with (G6-id+) hG6.3 with its BCR target and demonstrated that hG6.3 binds to germline encoded resides in VH complementary-determining region (CDR-H2). We have also isolated a number of potent CBIs and bi-specific Abs that will be studied. In Aim 1 we will continue to develop the B-CLL humanized mouse model and patient-derived organotypic spheroids (PDOTs) containing tumor and immune cells from these mice. We will use scRNASeq, immune receptor profiling, CITESeq and multiplex IHC to characterize the immune profile of these tumors to study the tumor microenvironment (TME) for both IGHV1-69+ and IGHV1-69− B CLL. In Aim 2, we will treat PDOTs with CASS B cells that secrete different CBIs and then conduct molecular profiling to determine the effects of different CBIs on the TME immune signatures. These ex vivo studies will result in a clear understanding of the linkage between CASS B cell activation and CBI secretion, while providing critical analysis of CASS B cell efficacy in comparison to CAR T cells at both a functional and molecular level. In Aim 3, we will treat humanized mice bearing B-CLL tumors with CASS B cells that show greatest recovery of anti-tumor immunity from Aim 2. scRNASeq and other interrogations described above will provide a detailed assessment of the molecular and mechanistic efficacy of the CBIs and CASS B cell platform as a whole. At the conclusion of this stage, we will have molecular insight into the B CLL TME and CASS B cell interventions that can optimally restore anti-B CLL immunity.

Checkpoint blockade inhibitor (CBI) monoclonal antibodies (mAbs) and adoptive cellular therapies have had a transformative effect on cancer therapies, changing the focus from simply killing tumor cells to activating a patients natural anti-tumor immunity and reversing the immunosuppressive tumor microenvironment (TME). While these represent some of the most promising anti-cancer therapeutics to date, only a small subset of patients experience complete or durable responses and many experience immune related adverse events (irAE) of varying severity. Another approach is the development of armored or immune restoring CAR T cells that are engineered to secrete an immunomodulatory payloads directly at the tumor site, increasing the efficacy while decreasing the on-target/off-tumor side effects seen with systemic delivery. In addition to T cells, various immune cells have been utilized for the creation of novel CARs, including natural killer cells (NK-CAR) and macrophages (CAR-M) and what unites all of these immune cell therapies is that they provide direct anti-tumor killing activity. A critical component of humoral immunity, B cells produce the antibodies on which the field of immunotherapy was originally developed. However, they possess no intrinsic cytotoxic capabilities and thus have been largely excluded from these advancements.

The Chimeric Antibody Signaling and Secretion (CASS) B cell platform is a novel and high-risk project as it seeks to advance B cell research to promote their participation into 21st century therapies. We will develop CASS B cell therapy to study and treat an aggressive type of treatment-resistant IGHV1-69-derived B CLL. CASS B cells are a unique B cell based cellular therapy that does not rely upon direct cytotoxicity but utilizes two intrinsic capabilities of B cells: the ability to secrete high levels of CBI antibodies to reverse the immunosuppressive TME and the ability to process and present antigens on MHC class II molecules, resulting in the recruitment of CD4+ T cells and allowing for enhanced tumor cell recognition and neoantigen spreading. While inducible, targeted delivery of the CBI will decrease irAEs, the ability to serve as a professional antigen presenting cells (APC) makes the CASS B cell platform unique in the cell therapy space, as CASS B cells have the power to initiate a robust anti-tumor response. MHC class II neoantigens played a function in innate anti-tumor responses. We will conduct both ex vivo and in vivo studies on humanized mice bearing B-CLL patient leukemic cells that are treated with CASS B cells secreting different CBI payloads that we discovered.

### Example 4

A role of B cells in our immune system is to recognize foreign invaders from microbes to cancer cells, and eliminate them by production of antibodies (Ab) that bind and clear the threat. B cells accomplish this by expressing a membrane bound Ab (B Cell Receptor BCRs) that binds the tumor antigen, causing the B cell to switch from a BCR expressing cell to an Ab secreting cell. After BCR engagement there is rapid tyrosine phosphorylation mediated by two primary tyrosine kinases, Lyn and Syn, and calcium ion polarization. These biochemical events lead to activation of downstream signaling pathways, resulting in further downstream activation of NF-κB and NFAT signaling and ultimately B cell expansion and robust mAb secretion. Without wishing to be bound by theory, we will engineer Chimeric Antibody Signaling and Secreting (CASS) B cells that will take advantage of these signaling pathways to induce clonal anti-tumor CASS B cell expansion and secretion of an anti-PDL1 Ab at the tumor site which will aid in restoring anti-tumor immunity.

Without wishing to be bound by theory, human B cells can be engineered to express artificial B cell receptors (aBCRs) against tumor associated antigens (TAAs) that will allow them to migrate to solid tumors where they will then be activated following aBCR binding to the TAA. In addition, once at the tumor site CASS B cells will be further engineered so that upon activation they will secrete an anti-PDL1 Ab that acts as a checkpoint blockade inhibitor (CBI) to reverse the immunosuppressive tumor microenvironment (TME).

In Aim 1 we will engineer B cells to express a membrane bound BCR of IgG type that is directed against the TAA carbonic anhydrase IX (CAIX) expressed on the surface of clear cell renal cell carcinoma (ccRCC). For proof-of-principle studies, a second Ab signaling plasmid containing a NFAT or NF-kB responsive element will be used to drive secretion of an anti-PDL1 Ab. Upon localization to the tumor site and BCR binding to CAIX, anti-PDL1 Ab secretion will be induced through NFAT or NF-kB activation. This activation should result in high anti-PDL1 Ab concentration at the tumor site. In Aim 2, we will use our orthotopic ccRCC mouse model to determine if anti-CAIX CASS B cells can migrate to the tumor and be activated by ccRCC to secrete anti-PDL1 Ab. We will also use this model to test safety of this therapy since CAIX is also expressed albeit at lower levels and with a different cytoplasmic distribution as compared to ccRCC. We have engineered anti-CAIX targeting moiety to preferentially recognize high-density CAIX on tumor cells but not healthy cholangiocytes.

Non-limiting Examples of Study Design: For Aim 1, we will construct and transfer the engineered anti-CAIX BCR into B cells using lentiviral transduction with expression driven from an internal spleen focus-forming virus (SFFV) promotor. Lentivirus particles will be pseudotyped with Gibbon-ape leukemia virus (GALV) or engineered baboon envelope glycoproteins to facilitate transduction. Separately we will design the reporter plasmid to allow for NFAT or NF-kB induction after BCR engagement to drive expression of GFP and the lead promoter will be used to drive anti-PDL mAb secretion. B cells will be transduced with both anti-CAIX BCR and NFAT/NF-kB inducible GFP lentiviral vectors and soluble CAIX-Fc will be used cross-link BCRs. GFP expression will then be measured to determine the optimal response element and the GFP will then be replaced with an anti-PDL1 mAb for secretion.

For Aim 2, CASS B cells will be constructed by transduction with the tumor specific anti-CAIX BCR and inducible anti-PDL1 mAb secretion lentiviral vectors and tested by mixing CASS B cells with CAIX+PDL1+ or PDL1− SKRC-59 ccRCC cells. In addition to measuring soluble anti-PDL1 secretion and binding to ccRCC cells, B cell activation will be measured by FACS for activation markers. In vivo experiments in humanized PBL NSG-SGM3 mice bearing SKRC-59 tumors will be used to evaluate CASS B cell efficacy and persistence. Efficacy will be tested by measuring secreted Ab and increased B cell population around the tumor site, and change in tumor size. To measure persistence, CASS B cells will be detected in both blood and the TME. Finally, scRNAseq will be used to analyze the effect of anti-PDL1 mAb on modulating the TME.

Without wishing to be bound by theory, compared to systemic delivery there will be a localized area of high anti-PDL1 antibody concentration centered around the tumor, leading to improved anti-tumor outcome and tumor elimination.

CASS B cells are a new concept in cellular therapy that will lead to restoration of local anti-tumor immunity in the TME by blocking the immunosuppressive PD-1/PD(L)-1 axis. It is also combination immunotherapy for the ease of a single administration and the cost of a single cellular infusion.

### Example 5

### Engineering Chimeric Antibody Signaling and Secreting (CASS) B Cells to Achieve Cancer Cures

Innovation: Cancer cells are a life form that has learned to commandeer our immune system for its own growth advantage. They do this through impairment of our cellular DNA repair mechanisms that lead to upregulation of growth factors and their receptors that results in uncontrolled tumor growth. This molecular high jacking also leads to surface expression and secretion of molecules involved in immune checkpoint blockade (ICB). Immunotherapy though anti-PD1/PDL1 blockade represents an important advance in the cancer field, and is a front-line or standard therapeutic option for various cancers, including non-small-cell lung cancer, melanoma, colorectal cancer, and renal cell carcinoma (1-3). However, while treatment successes are well documented, they most often do not result in cancer cures.

Cellular therapies are a way to harness the immune system to kill cancer cells. This is largely achieved by T cell receptor (TCR) and chimeric antigen receptor (CAR) T cells which are able to home to and target the cancer cells. While their success in treating solid tumors is improving, there are still limitations owing to the fact that cancer cells can also commandeer these T cells and render them ineffective.

Described herein is a new cellular therapy called Chimeric Antibody Signaling and Secreting (CASS) B cells that will utilize B cells of the humoral immune system to do what they do best which is secrete high levels of antibodies. These CASS B cells will be engineered to recognize tumor associated antigens (TAA) through an engineered B cell receptor (BCR), which upon TAA engagement will activate the CASS B cell and induce the production of high levels of an immunomodulatory bi-specific antibody (BsAb) at the tumor site. This will allow for reversal of the immunosuppressive tumor microenvironment as it does when immune checkpoint blockade monoclonal antibodies (mAbs) are delivered systemically. However, an interesting feature here is that BsAb secretion will be conditionally expressed only upon CASS B cell engagement with TAAs, and largely localized to the tumor site although some low level of leakage into the periphery will likely still occur. Without wishing to be bound by theory this is a step toward a cellular therapy where locally secreted monoclonal antibodies are devoted to changing the tumor microenvironment and restoring local anti-tumor immunity.

Rationale: Monoclonal antibody (mAb) drugs that directly kill cancer cells, act as immune checkpoint blockade modulators (e.g., inhibitors) or disrupt tumor vasculature are among the most promising anti-cancer therapeutics under development. However, the idea of engineering human B cells to seek out cancer cells and secrete these mAbs at the tumor site in vivo is novel and untested but could provide a powerful new way to treat both primary and metastatic tumors. It would also provide a lifelong anti-tumor immune surveillance system to prevent cancer reoccurrence and achieve “CURES”. The main role of B cells in our immune system is to recognize foreign invaders and eliminate them by production of antibodies that bind and clear the threat whether a microbe or cancer cell. They accomplish this by expressing a membrane bound antibody that acts as a B cell Receptor (BCRs) that bind the tumor antigen, causing the B cell to switch from a BCR expressing cell to an antibody secreting cell. After BCR engagement there is rapid tyrosine phosphorylation mediated by two primary tyrosine kinases, Lyn and Syn, and calcium ion polarization. These biochemical events lead to activation of downstream signaling pathways, resulting in further downstream activation of NF-kB and NFAT signaling and ultimately in B cell expansion and robust mAb secretion (5). We will engineer Chimeric Antibody Signaling and Secreting (CASS) B cells that will take advantage of these signaling pathways to induce clonal CASS B cell expansion and secretion of an immunomodulatory BsAb at the tumor site.

Without wishing to be bound by theory, the development of the CASS B cell platform can provide therapeutic benefit to a multitude of clinical indications that are sensitive to checkpoint blockade modulators (e.g., inhibitors) or have an immunosuppressive microenvironment. Anti-PD1/PDL1 therapy has had a transformative effect on the treatment of NSCLC in particular, becoming a frontline therapy for many patients. However, there are limitations to the efficacy, durability, and scope of use for this therapy. To counter these challenges, current trials are focusing on anti-PD1/anti-TIGIT combination therapies and Merck and Roche recently released clinical trial data indicating this combination shows considerable promise (6-7). We will use NSCLC as a model utilizing anti-PD1/anti-TIGIT bi-specific antibodies.

Objectives: To target the CASS B cell to the NSCLC tumor site, an engineered, membrane bound, single chain IgG BCR against mesothelin (MSLN) will be used. The plasmid will also contain a second cassette driven by a NFAT or NF-κB responsive element that drives secretion of an anti-TIGIT/anti-PD1 BsAb. Upon localization to the tumor site and BCR activation, BsAb expression will be induced by the native signaling pathway upon BCR engagement of MSLN on NSCLC. Since this will be an inducible expression system, there will be a localized area of high antibody concentration at the tumor site, significantly decreasing on-target, off-tumor effects.

Methods: The CASS B cell will be designed in two parts. Without wishing to be bound by theory we can transfer the engineered BCR into the B cell. To efficiently transduce B cells, lentivirus particles will be pseudotyped with Gibbon-ape leukemia virus (GALV) or engineered baboon envelope glycoproteins and in the transfer vector BCR expression will be driven from a spleen focus-forming virus (SFFV) or human elongation factor-1 alpha (EF1α) promotor (8). The second step will be to design a NFAT and/or NF-kB inducible expression cassette for BsAb secretion (9). To decide which response element to use, plasmids will be designed to express GFP. Raji cells and primary B cells will be transduced with the engineered anti-MSLN BCR and NFAT/NF-kB inducible GFP lentiviral vector, and soluble, biotinylated MSLN will be added to the culture media with streptavidin to cross-link BCRs. GFP expression will then be measured to determine the optimal response element. Preliminary work constructing an engineered membrane bound IgG (memIgG) was performed using an anti-influenza antibody. FIG. 1 shows that our memIgG construct expresses at high levels and is functionally active as it is able to bind soluble HA.

Next, CASS B cells will be constructed by lentivirus transduction with the vector encoding the tumor specific anti-MSLN BCR and an inducible BsAb replacing GFP. CASS B cells secretion of anti-TIGIT/PD1 BsAb will be quantified, first using soluble biotinylated MSLN+streptavidin to induce expression, followed by co-incubation with MSLN+ A549 NSCLC cells. Secondary experiments will test for in vitro inhibition of exhaustion by co-culturing CASS B cells with CD3+ T cells and A549 cells expressing various combinations of PDL1 and CD155 (ligand of TIGIT). In addition to measuring soluble antibody concentration, cellular activation/exhaustion will be measured by FACS staining. These tumor cells will also be stained for binding of CASS B cell shed anti-MSLN. In vivo experiments will next be carried out in humanized PBL NSG-SGM3 mice bearing A549 tumors to evaluate CASS B cell efficacy and persistence. Efficacy will be measured by local BsAb secretion by immunohistochemical staining, increased B cell population around the tumor site, and change in tumor size. We will also measure BsAb leakage into periphery by examining serum secreted BsAb concentration, to measure persistence, CASS B cells will be detected in both peripheral blood and the tumor microenvironment (TME). Finally, scRNAseq will be used to analyze the effect of the BsAb in modulating the TME. Ideally, compared to systemic delivery there will be a localized area of high BsAb concentration centered around the tumor, leading to improved outcome and tumor elimination.

Impact: The goal of developing new cancer therapies should be aimed at achieving “CURES”. Restoring host anti-tumor immunity at the tumor site is achievable by understanding the immune elements that are common or “public” to all individuals. Once this is done, we must develop an internal immunosurveillance system that will forever be present to prevent cancer from reoccurring. We will develop a new immune surveillance system using B cells of the immune system that have never been developed for this purpose. We will determine the feasibility of engineering Chimeric Antibody Signaling and Secreting (CASS) B cells to target mesothelin (MSLN) expressing NSCLC cells. These anti-MSLN CASS B cells will home to the tumor site, where they will secrete high levels of a bi-specific anti-TIGIT/anti-PDL1 antibody that will act as a dual checkpoint blockade inhibitor. This will result in a dynamic shift in the tumor microenvironment that will help reverse T cell exhaustion and restore anti-tumor immunity. An important and practical point is that this combination cellular immunotherapy will be a one-time administered and cost to the payer for the lifetime of the patient.

### REFERENCES CITED IN THIS EXAMPLE

1. Rolfo C, Caglevic C, Santarpia M, Araujo A, Giovannetti E, Gallardo C D, Pauwels P, Mahave M. Immunotherapy in NSCLC: A Promising and Revolutionary Weapon. Adv Exp Med Biol. 2017:995:97-125. doi: 10.1007/978-3-319-53156 4_5.PMID: 28321814 Review.
2. M.-O. Grimm, K. Leucht, V. Grunwald, S. Foller, New First Line Treatment Options of Clear Cell Renal Cell Cancer Patients with PD-1 or PD-L1 Immune-Checkpoint Inhibitor-Based Combination Therapies. J. Clin. Med. 9, 565 (2020).
3. X. Wu, et al., Application of PD-1 Blockade in Cancer Immunotherapy. Comput. Struct. Biotechnol. J. 17, 661-674 (2019).
4. Ryeong Lee B, Sehyun C, Moon J, Joon Kim M, Lee H, Wan Ko H, Chul Cho B, Sup Shim H, Hwang D, Ryun Kim H, and Ha S-J. Combination of PD-L1 and PVR determines sensitivity to PD-1 blockade. JCIinsite 2020: 5 (14): e128633. https://doi.org/10.1172/jci.insight.128633.
5. Tolar P, Hanna J, Krueger P D, Pierce S K. The constant region of the membrane immunoglobulin mediates B cell-receptor clustering and signaling in response to membrane antigens. Immunity. 2009: 30 (1): 44-55. Epub 2009 Jan. 13. doi: 10.1016/j.immuni.2008.11.007. PubMed PMID: 19135393; PMCID: PMC2656684.45.
6. Bendell J C, Bedard P, Bang Y J, et al: Phase Ia/Ib dose-escalation study of the anti-TIGIT antibody tiragolumab as a single agent and in combination with atezolizumab in patients with advanced solid tumors. 2020 AACR Virtual Annual Meeting II. Abstract CT302.
7. M.-J. Ahn, et al., 1400P Vibostolimab, an anti-TIGIT antibody, as monotherapy and in combination with pembrolizumab in anti-PD-1/PD-L1-refractory NSCLC. Ann. Oncol. 31, S887 (2020).
8. Levy C, Fusil F, Amirache F, Costa C, Girard-Gagnepain A, Negre D, Bernadin O, Garaulet G, Rodriguez A, Nair N, Vandendriessche T, Chuah M, Cosset F L, Verhoeven E. Baboon envelope pseudotyped lentiviral vectors efficiently transduce human B cells and allow active factor IX B cell secretion in vivo in NOD/SCIDgammac (−/−) mice. J Thromb Haemost. 2016; 14 (12): 2478-92. Epub 2016 Sep. 30. doi: 10.1111/jth. 13520. PubMed PMID: 27685947.
9. Uchibori R, Teruya T, Ido H, Ohmine K, Sehara Y, Urabe M, Mizukami H, Mineno J, Ozawa K. Functional Analysis of an Inducible Promoter Driven by Activation Signals from a Chimeric Antigen Receptor. Mol Ther Oncolytics. 2019; 12:16-25. Epub 2019 Jan. 22. doi: 10.1016/j.omto.2018.11.003. PubMed PMID: 30662937; PMCID: PMC6325072.

### Example 6

### CASS B Cell Culture and Transduction Protocol

### Day 0

1. Isolate B cells from fresh or frozen PBMCs using either (from Stemcell):
   19054 EasySep™ Human B Cell Enrichment Kit
   19554 EasySep™ Human Pan-B Cell Enrichment Kit (includes plasma cells)
2. Activate B cells, such as between ˜18-24 hours and 3-5 days, prior to transduction
   a. Typically culture in 24 well plate, 1 ml media with 0.5-1E6 cells/ml
   Activation media 1 (used protocol for Milteyni 130-106-196 but added 1 μg/ml ODN2006): Milteyni StemMACS HSC media+5% FBS+IL4+ multimerized CD40L+ODN2006
Activation media 2 (Based off of Moffett et al DOI 10.1126/sciimmunol.aax0644): IMDM+10% FBS+CD40L-Fc (3.735 ug/ml)+ODN2006 (1 ug/ml)+IL2 (50 ng/ml)+IL10 (50 ng/ml)+IL15 (10 ng/ml)
Originally used activation media 1, but have since changed to activation media 2

### Day 1

1. Thaw virus
2. Add DEAE at final concentration of 10 μg/ml to each well
3. Add virus and pipette to mix
4. Centrifuge virus at 1200×g for 55 min at 37C
5. Transfer plate to incubator and allow to rest overnight, do not need to resuspend the cells

### Day 2

1. Collect cells in 15 ml tube and was 2× with PBS prior to resuspending in fresh activation media
   a. Ideally will be able to culture in ˜1 ml at 1E6 cells/ml but resuspend accordingly based on number of cells and viability
   b. Maintaining cells at 0.75-1E6 cells/ml is important for rapid expansion during this stage
2. Incubate cells for 48 hours to allow for expression of transduced genes and expansion of cells prior to sorting

### Day 4

1. In morning, using fresh or frozen irradiated 3T3-msCD40L feeder cells (HIV Reagents #12535)
   a. cells γ-irradiated at 78 Gy
2. Plate 8.75E5 cells/cm² in DMEM+10% FBS, allow cells to attach before sorting (>4 hours)
3. Collect cells, wash with PBS, and stain with Zombie Violet (live/dead exclusion dye from Biolegend)
   a. Follow manufacturer's protocol, 1:1000 dilution of dye, 100 ul diluted dye/1E6 cells
4. Block Fc receptors with Human TruStain FcX (Biolegend 422302)
5. Stain cells with appropriate markers for sorting
   a. QBend10-PE, CD19-APC, Zombie Violet uses BFP channel
6. Aspirate media from 6 well plate with adherent 3T3-msCD40L feeder cells and replace with expansion media (IMDM+10% FBS+5 μg/ml hInsulin+50 μg/ml transferrin+50 ng/ml IL2+10 ng/ml IL15+20 ng/ml IL21)
7. Sort cells on Sony MA900 directly into 6 well plate with feeder cells
8. Culture cells for 14+ days, add media as appropriate. If culturing for over 7 days, split into fresh plate with irradiated feeder cells on Day 6 or 7

### Example 7

Exemplary constructs comprise:
(1) EF1alpha-F105leader-F10-memIgG1-T2A-RQR8-3×3NFAT-minIL2pro-ZsG,
(2) EF1alpha-F105leader-F10-memIgG1-T2A-RQR8-5×NFAT-minIL2pro-ZsG,
(3) EF1alpha-F105leader-F10-memIgG1-T2A-RQR8-NFAT-NFκB-ZsG, or
(4) any combination thereof.

### EF1alpha Promoter:

(SEQ ID NO: 7)
CGTGAGGCTCCGGTGCCCGTCAGTGGGCAGAGCGCACATCGCCCACAGTC
CCCGAGAAGTTGGGGGGAGGGGTCGGCAATTGAACCGGTGCCTAGAGAAG
GTGGCGCGGGGTAAACTGGGAAAGTGATGTCGTGTACTGGCTCCGCCTTT
TTCCCGAGGGTGGGGGAGAACCGTATATAAGTGCAGTAGTCGCCGTGAAC
GTTCTTTTTCGCAACGGGTTTGCCGCCAGAACACAGGTAAGTGCCGTGTG
TGGTTCCCGCGGGCCTGGCCTCTTTACGGGTTATGGCCCTTGCGTGCCTT
GAATTACTTCCACGCCCCTGGCTGCAGTACGTGATTCTTGATCCCGAGCT
TCGGGTTGGAAGTGGGTGGGAGAGTTCGAGGCCTTGCGCTTAAGGAGCCC
CTTCGCCTCGTGCTTGAGTTGAGGCCTGGCCTGGGCGCTGGGGCCGCCGC
GTGCGAATCTGGTGGCACCTTCGCGCCTGTCTCGCTGCTTTCGATAAGTC
TCTAGCCATTTAAAATTTTTGATGACCTGCTGCGACGCTTTTTTTCTGGC
AAGATAGTCTTGTAAATGCGGGCCAAGATCTGCACACTGGTATTTCGGTT
TTTGGGGCCGCGGGCGGCGACGGGGCCCGTGCGTCCCAGCGCACATGTTC
GGCGAGGCGGGGCCTGCGAGCGCGGCCACCGAGAATCGGACGGGGGTAGT
CTCAAGCTGGCCGGCCTGCTCTGGTGCCTGGCCTCGCGCCGCCGTGTATC
GCCCCGCCCTGGGCGGCAAGGCTGGCCCGGTCGGCACCAGTTGCGTGAGC
GGAAAGATGGCCGCTTCCCGGCCCTGCTGCAGGGAGCTCAAAATGGAGGA
CGCGGCGCTCGGGAGAGCGGGCGGGTGAGTCACCCACACAAAGGAAAAGG
GCCTTTCCGTCCTCAGCCGTCGCTTCATGTGACTCCACGGAGTACCGGGC
GCCGTCCAGGCACCTCGATTAGTTCTCGAGCTTTTGGAGTACGTCGTCTT
TAGGTTGGGGGGAGGGGTTTTATGCGATGGAGTTTCCCCACACTGAGTGG
GTGGAGACTGAAGTTAGGCCAGCTTGGCACTTGATGTAATTCTCCTTGGA
ATTTGCCCTTTTTGAGTTTGGATCTTGGTTCATTCTCAAGCCTCAGACAG
TGGTTCAAAGTTTTTTTCTTCCATTTCAGGTGTCGTGA

### F105 Leader Sequence:

| (SEQ ID NO: 8) | |
|---|---|
| | ATGAAACATCTGTGGTTCTTCCTTCTCCTGGTGGCAGCGGCCCAG |

F10 scFv:

(SEQ ID NO: 9)
QVQLVQSGAEVKKPGSSVKVSCTSSEVTFSSFAISWVRQAPGQGLEWLGG
ISPMFGTPNYAQKFQGRVTITADQSTRTAYMDLRSLRSEDTAVYYCARSP
SYICSGGTCVEDHWGQGTLVTVSSGGGGSGGGGSGGGGIQPGLTQPPSVS
KGLRQTATLTCTGNSNNVGNQGAAWLQQHQGHPPKLLSYRNNDRPSGISE
RFSASRSGNTASLTITGLQPEDEADYYCSTWDSSLSAVVFGGGTKLTVLG
QPKAAPSAAAE

### Fc Domain (Bold is CH2, Underlined is CH3)

(SEQ ID NO: 10)
PELLGGPSVFLFPPKPKDTLMISRTPEVTCVVVDVSHEDPEVKENWYVDG
VEVHNAKTKPREEQYNSTYRVVSVLTVLHQDWLNGKEYKCKVSNKALPAP
IEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEA
LHNHYTQKSLSLSP

### Transmembrane and Intracellular Domain:

(SEQ ID NO: 11)
GLWTTITIFITLFLLSVCYSATVTFFKVKWIFSSVVDLKQTIIPDYRNMI
GQGAP

### T2A:

| (SEQ ID NO: 12) | |
|---|---|
| | GSGEGRGSLLTCGDVEENPGP |

### RQR8:

(SEQ ID NO: 13)
MGTSLLCWMALCLLGADHADACPYSNPSLCSGGGGSELPTQGTFSNVSTN
VSPAKPTTTACPYSNPSLCSGGGGSPAPRPPTPAPTIASQPLSLRPEACR
PAAGGAVHTRGLDFACDIYIWAPLAGTCGVLLLSLVITLYCNHRNRRRVC
KCPRPVV

### Z&G:

(SEQ ID NO: 14)
MAQSKHGLTKEMTMKYRMEGCVDGHKFVITGEGIGYPFKGKQAINLCVVE
GGPLPFAEDILSAAFMYGNRVFTEYPQDIVDYFKNSCPAGYTWDRSFLFE
DGAVCICNADITVSVEENCMYHESKEYGVNFPADGPVMKKMTDNWEPSCE
KIIPVPKQGILKGDVSMYLLLKDGGRLRCQFDTVYKAKSVPRKMPDWHFI
QHKLTREDRSDAKNQKWHLTEHAIASGSALP

NFAT Binds to a 9 bp Element with the Consensus Sequence

(A/T)GGAAA(A/N)(A/T/C)N, wherein N represents any
base

### 3×NFAT RE:

| (SEQ ID NO: 15) | |
|---|---|
| | TGGAAAGATTGGAAAGACTGGAAAGAT |
| (SEQ ID NO: 16) | |
| | TGGAAAGAGAGGAAACACTGGAAAGAG |
| (SEQ ID NO: 17) | |
| | TGGAAAGATAGGAAACACTGGAAAGAG |

### 5×NFAT RE:

| (SEQ ID NO: 18) | |
|---|---|
| | TGGAAAGATTGGAAAGACTGGAAAGATAGGAAACACTGGAAAGAG |

### IL8 NFAT RE:

GAGGAATTT

minIL2 Prometer:

(SEQ ID NO: 19)
GAGCCCGGGACATTTTGACACCCCCATAATATTTTTCCAGAATTAACAGT
ATAAATTGCATCTCTTGTTCAAGAGTTCCCTATCACTCTCTTTAATCACT
ACTCACAGTAACCTCAACTCCTGCATTCCATGGGTTGGATCCGCG

minIL8 Promoter:

| (SEQ ID NO: 20) | |
|---|---|
| | TAGAGGGTATATAATGGAAGCTCGACTTCCA |

### WPRE:

(SEQ ID NO: 21)
TAATCAACCTCTGGATTACAAAATTTGTGAAAGATTGACTGGTATTCTTA
ACTATGTTGCTCCTTTTACGCTATGTGGATACGCTGCTTTAATGCCTTTG
TATCATGCTATTGCTTCCCGTATGGCTTTCATTTTCTCCTCCTTGTATAA
ATCCTGGTTGCTGTCTCTTTATGAGGAGTTGTGGCCCGTTGTCAGGCAAC
GTGGCGTGGTGTGCACTGTGTTTGCTGACGCAACCCCCACTGGTTGGGGC
ATTGCCACCACCTGTCAGCTCCTTTCCGGGACTTTCGCTTTCCCCCTCCC
TATTGCCACGGCGGAACTCATCGCCGCCTGCCTTGCCCGCTGCTGGACAG
GGGCTCGGCTGTTGGGCACTGACAATTCCGTGGTGTTGTCGGGGAAATCA
TCGTCCTTTCCTTGGCTGCTCGCCTGTGTTGCCACCTGGATTCTGCGCGG
GACGTCCTTCTGCTACGTCCCTTCGGCCCTCAATCCAGCGGACCTTCCTT
CCCGCGGCCTGCTGCCGGCTCTGCGGCCTCTTCCGCGTCTTCGCCTTCGC
CCTCAGACGAGTCGGATCTCCCTTTGGGCCGCCTCCCCGCCT

### Example 8

### Engineering Chimeric Antibody Signaling and Secreting (CASS) B Cells to Achieve Cancer Cures

### Abstract

B cells are a component of host immunity and while other immune cells including T cells, NK cells, and myeloid cells have been engineered with Chimeric Antigen Receptors (CAR), B cells have been ignored. The role of B cells in our immune system is to recognize foreign invaders via a membrane bound antibody (B cell receptor, BCR) and eliminate them by producing antibodies that bind and clear the threat. We can develop the Chimeric Antibody Signaling and Secreting (CASS) B cell platform by engineering both the target binding domain and secreted antibody to develop a potent anti-cancer therapeutic drug. The target binding domain will be designed to recognize a tumor associated antigen and upon binding will lead to the secretion of a bi-specific checkpoint blockade inhibiting (CBI) antibody to reverse exhaustion of immune cells that have arrived at the tumor site. A second major function of B cells is to present foreign protein fragments to T cells, leading to their activation and the further recruitment of various anti-tumor immune cells. Lastly, B cells play a role in immunologic memory and CASS B cells will provide a lifelong anti-tumor surveillance network protecting patients from metastasis and reoccurrence after the initial tumor is eliminated.

While the CASS B cell platform is applicable to a number of cancers, we will use non-small cell lung cancer (NSCLC) as an experimental model. NSCLC was chosen because it can express high levels the tumor associated antigen, mesothelin (MSLN), and because of the effectiveness of anti-PD1 and anti-TIGIT antibody combination therapies seen in clinical trials. Aim 1 will focus on engineering the CASS B cell, first by building the synthetic B cell receptor (BCR) to recognize MSLN. The cells will be further engineered to express a bi-specific antibody targeting PD1 and TIGIT under control of a switch that is only activated upon MSLN binding at the tumor site. The second aim will use tissue culture experiments to support that our engineering was successful and to indicate the capability of the bi-specific antibody to restore anti-cancer immunity. Experiments in Aim 3 will be performed using mice possessing a human immune system to demonstrate the effectiveness of the CASS B cell platform in vivo and allow for detailed molecular characterization of the anti-tumor immune response. Without wishing to be bound by theory, the development of the CASS B cell platform supports translational research for the development of new therapeutic platforms that can impact a wide range of clinical indications and have had a decisive role in improving health care and saving lives.

### Scientific Abstract

Immune checkpoint blockade inhibitors (CBI) and CAR T cells have revolutionized the way we treat cancer. While both of these therapies engage the patient's immune system, with CBI therapies inhibiting immunosuppressive signals generated by the tumor microenvironment and activated CAR T cells engaging bystander cells via the release of stimulatory and proinflammatory cytokines, neither is able to proactively engage and initiate an anti-tumor immune response. To address this, we can develop Chimeric Antibody Secreting and Signaling (CASS) B cells, which express an engineered, tumor associated antigen targeting BCR and upon engagement, will secrete high levels of a bi-specific antibody (bsAb) locally at the tumor site that acts as a CBI. As B cells are also professional antigen presenting cells (APCs), they will process and present additional tumor antigens on MHC class II molecules, further enhancing tumor cell recognition and assisting in neoantigen spreading. As a component of immunologic memory, CASS B cells will provide a lifelong surveillance program protecting against tumor metastasis and recurrence. Driven by the recruitment of a wide range of immune cells to the tumor site and reversal the immunosuppressive tumor microenvironment allowing these cells to function, CASS B cell therapy will provide a robust and durable anti-cancer therapeutic.

Mesothelin (MSLN) is a tumor associated antigen due to its limited expression on healthy tissue and numerous biologic therapies targeting this marker have been devised, including CAR T and recombinant antibodies. Non-small cell lung cancer (NSCLC) have been indicated to upregulate MSLN expression and data from clinical trials have demonstrated the effectiveness of anti-PD1/anti-TIGIT antibody combination therapies. Based off of this, NSCLC was chosen for a model to develop and test the efficacy of an anti-MSLN directed CASS B cell secreting an immunomodulatory anti-PD1/TIGIT bsAb. Aim 1 will focus on the development of the CASS B cell platform and engineering of the BCR to recognize mesothelin (MSLN). Additional engineering will be performed to use the native BCR signaling pathways to develop an inducible system such that MSLN engagement at the tumor site leads to high levels of bsAb secretion. In Aim 2 the in vitro efficacy of the CASS B cell will be tested, and a characterization of the activation thresholds and resultant secretion levels will be performed to allow for fine tuning of CASS B cell signaling and secretion. Aim 3 will execute in vivo experiments using both a NSCLC cell line model and a patient derived xenograft model in mice reconstituted with human immune systems. Multiparameter flow cytometry (FACS), single cell RNA sequencing, and immunohistochemistry will provide a detailed assessment of the molecular and mechanistic efficacy of the immunomodulatory bsAb and the CASS B cell platform as a whole.

Without wishing to be bound by theory, the development of the CASS B cell platform supports innovative translational research for the development of new therapeutic platforms that can impact a wide range of clinical indications. While this example focuses on NSCLC, this research program will enable our team to further refine this new translational approach and expand it beyond cancer therapies. The development of the CASS B cell platform has potential therapeutic benefit not only in cancer therapies, but a multitude of other clinical indications, including autoimmune/rheumatic and cardiovascular diseases and neurologic disorders where mAb therapies have had a decisive role in improving health care and saving lives.

### Non-Limiting Unique and Innovative Aspects

The FDA approved the first biologic (Humulin) in 1982 followed by the first monoclonal antibody therapy (muromonab-CD3) in 1986, and in the years since, we have seen an explosion of new biologics. After the initial success of anti-PD1 and CTLA4 therapies, the race has been on how to develop antibodies against the next generation of checkpoint molecules. As researchers began to identify new biomarkers, it became apparent that it would be difficult to find a receptor that rivals PD1 and CTLA4 and these alone would not be sufficient for most patients. As such, the field moved towards combination therapies to combine anti-PD(L)1 therapies with a wide range of other compounds, both standard of care and experimental. These antibody-based therapies rely on restoring anti-tumor activity of the patient's immune system, however different cancers commandeer a diversity of checkpoint blockade pathways to evade our immune system and not all cancers respond to the same immunotherapies. The next breakthrough came with the development of CAR T cell therapies, where patient T cells are removed and engineered to recognize a tumor associated antigen, allowing for robust targeting and cytotoxic activity. While CAR T activity indirectly activates other immune cells via cytokine and chemokine release, a significant cytotoxic effect is derived from the CAR T cell. Additionally, as CAR T cells are “living drugs” they will expand and become part of the patients' immune system, providing long term protection not achieved with monoclonal antibody therapy.

Chimeric Antibody Secreting and Signaling (CASS) B cells are a new form of cellular therapy that uses engineered B cells to secrete high levels of checkpoint blockade modulator antibodies (e.g., checkpoint inhibitor antibodies) locally at the tumor site. One of the challenges with systemic delivery of CBI antibody therapies is widespread target distribution and the role these receptors play in immune homeostasis, potentially leading to immune related adverse events (irAE) of varying severity including colitis, dermatitis, myocarditis, encephalitis, or peripheral neuropathy. To overcome this challenge, targeted delivery of CBI therapies are being explored by a number of groups, where CAR T cells are engineered to secrete therapeutic payloads at the tumor site, increasing the local concentrations relative the serum concentrations. A unique aspect here is that by utilizing the native BCR signaling pathways, the engineered IgG-BCR is designed to selectively induce high levels of CBI bsAb expression only upon activation by the target antigen, creating a pocket of high bsAb concentration and reversal of immune suppression surrounding the tumor. The use of a bsAb is also an innovative solution to improve on the efficacy seen by mono and combination antibody therapies as by tethering the binding domains together, potential synergistic activity can be gained. As with CAR T cells, CASS B cells will serve as a living drug and become a permanent part of the patients' immune system, providing lifelong immunosurveillance and protection from metastasis and reoccurrence.

While CASS B cells do not offer the direct cytotoxic effect seen by CAR T cells, upon stimulation, B cells process and present antigens on MHC class II molecules, recruiting CD4+ T cells and allowing for enhanced tumor cell recognition and neoantigen spreading. The antigen presenting capabilities of the CASS B cell platform make it unique in the cell therapy space. As with antibody-based therapies CASS B cells require activation of a patient's immune system, however as professional antigen presenting cells, CASS B cells have the power to initiate a robust anti-tumor response by engaging a broad set of immune cells. Another critical role of B cells in the immune system is that memory B cells are a key complement to immunologic memory, and upon restimulation are able to rapidly produce large amounts of antibody. This provides a means for life-long surveillance for tumor metastasis and reoccurrence.

Building off the Marasco Lab's expertise in antibody engineering and CAR T cell development, the CASS B cell platform can be a new investigational pathway. Our work has provided extensive experience in the discovery and engineering of therapeutic quality antibodies against a diverse list of targets including viral and tumor associated antigens, as well as numerous immune checkpoint markers⁷⁻¹¹. Additionally, we have used these antibodies to develop a series of bi-specific and multispecific antibody constructs using knob-in-hole, IgG fusions, and tandem scFvs. CAR T cells and the development of our CAR T cell factories provided the lab's first foray into cellular therapies and targeted delivery of immunomodulatory antibodies to the tumor site². These CAR T programs have provided the lab with valuable experience and pipeline development for the evaluation and analysis of biologic therapies in vitro and in vivo. Skills and techniques gained from these previous lines of research will provide the groundwork allowing our lab to pursue the new investigational path of CASS B cell development.

Since the first edition CAR T cells developed in the early 1990s, significant improvements in efficacy, persistence, and safety have led to the creation of the 2^{nd} and 3^{rd} generation CAR T cells in use today. Other immune cells have been utilized for the creation of new CARs, including natural killer cells (NK-CAR) and macrophages (CAR-M) and what unites all of these cells is that they provide direct anti-tumor activity^{12,13}. Though B cells are a critical component of humoral immunity and produce the antibodies on which the field of immunotherapy was originally developed, they possess no intrinsic cytotoxic capabilities of their own and thus have been largely excluded from these advancements. The CASS B cell platform seeks to bring B cell research into the 21^{st} century by developing a unique B cell based cellular therapy that does not rely upon direct cytotoxicity but rather utilizes the antibody secreting function of B cells to reverse immunosuppressive environments combined with the antigen presenting function to activate and recruit additional immune effector cells to eliminate the tumor.

While this example focuses on the use of CASS B cells for the treatment of NSCLC, the CASS B cell described in this proposal is therapeutically applicable for a multitude of MSLN+ cancers with an immunosuppressive nature, including ovarian and pancreatic cancers. With its modular design, CASS B cells are readily directed to other tumor associated antigens and the secreted payload can be adjusted to target the relevant immunologic axis, allowing for the adaptation of CASS B cells to a wide variety of cancers. As B cells are long lived and an important part of immunologic memory, CASS B cells continuously deliver the therapeutic payload at the tumor site and after the primary tumor has been eradicated, providing a lifelong immunosurveillance system against metastasis and reoccurrence. The long duration and inducible nature of the CASS B cell platform can be readily adapted to other diseases treated with biologic therapies, such as cardiovascular and autoimmune diseases and neurological disorders, which typically require long term disease maintenance and rapid administration of therapeutics to treat acute symptoms. Therefore, the development of the CASS B cell platform can benefit not only cancer research, but a diverse range of clinical indications.

### Example 9

### Chimeric Antibody Signaling and Secreting (CASS) B Cells

1. Constitutive expression of an a-tumor antigen synthetic BCR
2. Inducible expression of an immunomodulatory payload
   Checkpoint blockade therapy (α-PD(L)1, α-PD1/TIGIT)
   T Cell engager (α-CD3×MSLN, α-CD3×CD28×MSLN)
   Cytokine fusions (α-PD1-scIL12)

### B Cell Basics:

B cell isolation: Stemcell EasySep™ Human Pan-B Cell Enrichment Kit

Stimulation Media: IMDM+10% HI FBS+CD40L-Fc (3.735 ug/ml)+ODN2006 (1 ug/ml)+IL2 (50 ng/ml)+IL10 (50 ng/ml)+IL15 (10 ng/ml)

Long term expansion media: IMDM+10% FBS+hInsulin (5 ug/ml)+transferrin (50 ug/ml)+IL2 (50 ng/ml)+IL21 (20 ng/ml)+IL15 (10 ng/ml)

Transduction Check after Removing BGH
Isolate and activate B cells: Nov. 11, 2021
Transduce: Nov. 12, 2021
Stain: Nov. 19, 2021

Virus was pseudotyped with VSVG and the BGH was removed. B cells were transduced 18-24 hours post activation. However, they are negative 7 days post transduction

Transduction Check with Our Vectors
Vectors:
LeGO-hEuMAR-GFP
pHAGE-F10-RQR8
pHAGE-F10-RQR8 NFAT/NFκB
B cell stimulation (4 days):
Media: IMDM+10% HI FBS+CD40L-Fc (3.735 ug/ml)+ODN2006 (1 ug/ml)+IL2 (50 ng/ml)+IL10 (50 ng/ml)+IL15 (10 ng/ml)
B cell transduction: Use GALV and BaEV envelopes without retronection

### EQUIVALENTS

Those skilled in the art will recognize, or be able to ascertain, using no more than routine experimentation, numerous equivalents to the specific substances and procedures described herein. Such equivalents are considered to be within the scope of this invention. and are covered by the following claims.

1. A genetically engineered B cell, wherein the genetically engineered B cell expresses and bears on its surface a chimeric B cell receptor, and wherein the genetically engineered B cell further expresses and secretes an antibody or cytokine.

2. The genetically engineered B cell of claim 1, wherein the chimeric B cell receptor comprises an extracellular domain, a transmembrane domain, and an intracellular signaling domain.

3. The genetically engineered B cell of claim 1, wherein the extracellular domain is an antibody or antibody fragment.

4. The genetically engineered B cell of claim 3, wherein the antibody is a nanobody, scFv, bi-specific antibody or Fab.

5. The genetically engineered B cell of claim 3, wherein the antibody is specific for a tumor associated antigen.

6. The genetically engineered B cell of claim 5, wherein the tumor associated antigen is selected from the group consisting of CAIX, BCMA, CD138, PD-L1, PD-L2, VEGF, CD70, CD99, CEA, Her-2, GD2, CD171, αFR, PMSA, IL13α, MSLN, TAG-72, and TROP2.

7. The genetically engineered B cell of claim 3, wherein the antibody is an anti-IGHV 1-69 antibody.

8. The genetically engineered B cell of claim 3, wherein the antibody is specific for an infectious disease associated antigen.

9. The genetically engineered B cell of claim 8, wherein the infectious disease is a viral disease.

10. The genetically engineered B cell of claim 8, wherein the infectious disease comprises influenza, coronavirus, HIV, or tuberculosis.

11. The genetically engineered B cell of claim 8, wherein the infectious disease associated antigen comprises HA1, HA2, NA, or spike protein.

12. The genetically engineered B cell of claim 1, wherein expression of the antibody or cytokine is controlled by an inducible response element.

13. The genetically engineered B cell of claim 12, wherein the inducible response element is an NFAT or NFκB response element.

14. The genetically engineered B cell of claim 1, wherein the antibody is a checkpoint blockade modulator.

15. The genetically engineered B cell of claim 14, wherein the antibody is a checkpoint blockade inhibitor.

16. The genetically engineered B cell of claim 1, wherein the antibody is specific for CA-9, PD-1, PD-L1, PD-L2, CTLA4, TIGIT, VISTA, CD70, TIM-3, LAG-3, CD40L, CCR4, GITR, or CXCR4.

17. The genetically engineered B cell of claim 1, wherein the cytokine is selected from the group consisting of IL-2, IL-7, IL-12, IL-15, IL-18, CD40-L, or BAFF.

18. The genetically engineered B cell of claim 1, wherein the antibody is specific for HA1, HA2, NA, or spike protein.

19. The genetically engineered B cell of claim 1, wherein the antibody comprises a monoclonal antibody.

20. The genetically engineered B cell of claim 1, wherein the antibody comprises a nanobody, scFv, Fab, antibody-cytokine fusion protein, or a bi-specific antibody.

21. The genetically engineered cell of claim 1, wherein the antibody comprises a humanized antibody.

22. The genetically engineered B cell of claim 20, wherein the antibody-cytokine fusion protein comprises anti-PD1-scIL 12.

23. The genetically B engineered cell of claim 20, wherein the bi-specific antibody is specific for PD-1 and CTLA4, PD-1 and TIGIT, TIGIT and CCR4, GITR and TIGIT, or PD-1 and CCR4.

24. A nucleic acid encoding the chimeric B cell receptor and the antibody or cytokine according to claim 1.

25.-40. (canceled)

41. A vector comprising the nucleic acid of claim 24.

42. The vector of claim 41, wherein the vector is a lentiviral vector or an adeno-associated virus vector.

43. A cell comprising the vector of claim 41.

44. A composition comprising the genetically engineered B cell of claim 1.

45.-66. (canceled)

67. A method of making a population of genetically engineered B cells, the method comprising:
isolating a population of B cells from a subject, and
transducing the population of B cells with the nucleic acid of claim 24,
thereby producing a population of genetically engineered B cells.

68. The method of claim 67, further comprising the step of activating the population of B cells prior to transduction.

69. The method of claim 67, further comprising the step of culturing the population of genetically engineered B cells.

70. The method of claim 67, further comprising the step of administering the population of genetically engineered B cells to a subject in need thereof.

71. A method of treating a subject afflicted with cancer, the method comprising administering to a subject the genetically engineered B cell of claim 1.

72. A method of preventing cancer in a subject, the method comprising administering to a subject the genetically engineered B cell of claim 1.

73. The method of claim 67, wherein the cancer is BCLL, NSCLC, ccRCC, mesothelioma.

74. A method of treating a subject afflicted with an infectious disease, the method comprising administering to a subject the genetically engineered B cell of claim 1.

75. A method of preventing an infectious disease, the method comprising administering to a subject the genetically engineered B cell of claim 1.

76. The method of claim 74, wherein the infectious disease is a viral disease.

77. The method of claim 74, wherein the infectious disease comprises influenza, coronavirus, HIV, or tuberculosis.


SEQUENCE LISTING (40 sequences)

SEQ ID NO: 1 - protein, 24 aa, synthetic construct
  VARIANT 5..24: note = GGGS repeats may be deleted
GGGSGGGSGGGSGGGSGGGSGGGS

SEQ ID NO: 2 - protein, 30 aa, synthetic construct
  VARIANT 6..30: note = GGGGS repeats may be deleted
GGGGSGGGGSGGGGSGGGGSGGGGSGGGGS

SEQ ID NO: 3 - protein, 12 aa, synthetic construct
GSAGSAAGSGEF

SEQ ID NO: 4 - protein, 4 aa, synthetic construct
GGGS

SEQ ID NO: 5 - protein, 7 aa, synthetic construct
GGGGSSS

SEQ ID NO: 6 - protein, 9 aa, synthetic construct
GGGGSSSSS

SEQ ID NO: 7 - DNA, 1188 bp, synthetic construct
  source 1..1188: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 8 - DNA, 45 bp, synthetic construct
  source 1..45: mol_type = other DNA
atgaaacatctgtggttcttccttctcctggtggcagcggcccag

SEQ ID NO: 9 - protein, 261 aa, synthetic construct
QVQLVQSGAEVKKPGSSVKVSCTSSEVTFSSFAISWVRQAPGQGLEWLGGISPMFGTPNYAQKFQGRVTITADQSTRTAYMDLRSLRSEDTAVYYCARSPSYICSGGTCVFDHWGQGTLVTVSSGGGGSGGGGSGGGGIQPGLTQPPSVSKGLRQTATLTCTGNSNNVGNQGAAWLQQHQGHPPKLLSYRNNDRPSGISERFSASRSGNTASLTITGLQPEDEADYYCSTWDSSLSAVVFGGGTKLTVLGQPKAAPSAAAE

SEQ ID NO: 10 - protein, 214 aa, synthetic construct
PELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 11 - protein, 55 aa, synthetic construct
GLWTTITIFITLFLLSVCYSATVTFFKVKWIFSSVVDLKQTIIPDYRNMIGQGAP

SEQ ID NO: 12 - protein, 21 aa, synthetic construct
GSGEGRGSLLTCGDVEENPGP

SEQ ID NO: 13 - protein, 157 aa, synthetic construct
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 14 - protein, 231 aa, synthetic construct
MAQSKHGLTKEMTMKYRMEGCVDGHKFVITGEGIGYPFKGKQAINLCVVEGGPLPFAEDILSAAFMYGNRVFTEYPQDIVDYFKNSCPAGYTWDRSFLFEDGAVCICNADITVSVEENCMYHESKFYGVNFPADGPVMKKMTDNWEPSCEKIIPVPKQGILKGDVSMYLLLKDGGRLRCQFDTVYKAKSVPRKMPDWHFIQHKLTREDRSDAKNQKWHLTEHAIASGSALP

SEQ ID NO: 15 - DNA, 27 bp, synthetic construct
  source 1..27: mol_type = other DNA
tggaaagattggaaagactggaaagat

SEQ ID NO: 16 - DNA, 27 bp, synthetic construct
  source 1..27: mol_type = other DNA
tggaaagagaggaaacactggaaagag

SEQ ID NO: 17 - DNA, 27 bp, synthetic construct
  source 1..27: mol_type = other DNA
tggaaagataggaaacactggaaagag

SEQ ID NO: 18 - DNA, 45 bp, synthetic construct
  source 1..45: mol_type = other DNA
tggaaagattggaaagactggaaagataggaaacactggaaagag

SEQ ID NO: 19 - DNA, 145 bp, synthetic construct
  source 1..145: mol_type = other DNA
gagcccgggacattttgacacccccataatatttttccagaattaacagtataaattgcatctcttgttcaagagttccctatcactctctttaatcactactcacagtaacctcaactcctgcattccatgggttggatccgcg

SEQ ID NO: 20 - DNA, 31 bp, synthetic construct
  source 1..31: mol_type = other DNA
tagagggtatataatggaagctcgacttcca

SEQ ID NO: 21 - DNA, 592 bp, synthetic construct
  source 1..592: mol_type = other DNA
taatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcct

SEQ ID NO: 22 - protein, 6 aa, synthetic construct
HHHHHH

SEQ ID NO: 23 - DNA, 1201 bp, synthetic construct
  source 1..1201: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaggcgcgccacc

SEQ ID NO: 24 - protein, 764 aa, synthetic construct
MKHLWFFLLLVAAAQPAMAQVQLVQSGAEVKKPGSSVKVSCTSSEVTFSSFAISWVRQAPGQGLEWLGGISPMFGTPNYAQKFQGRVTITADQSTRTAYMDLRSLRSEDTAVYYCARSPSYICSGGTCVFDHWGQGTLVTVSSGGGGSGGGGSGGGGIQPGLTQPPSVSKGLRQTATLTCTGNSNNVGNQGAAWLQQHQGHPPKLLSYRNNDRPSGISERFSASRSGNTASLTITGLQPEDEADYYCSTWDSSLSAVVFGGGTKLTVLGQPKAAPSAAAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPELQLEESCAEAQDGELDGLWTTITIFITLFLLSVCYSATVTFFKVKWIFSSVVDLKQTIIPDYRNMIGQGAPLINGSGEGRGSLLTCGDVEENPGPTRMGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 25 - DNA, 320 bp, synthetic construct
  source 1..320: mol_type = other DNA
taagtatgatcagcctcgagctagctggaaagattggaaagactggaaagatccattagatcgtttggaaagagaggaaacactggaaagagtcagcctcataggtggaaagataggaaacactggaaagagccattagatctcgagtggaagcttagacacgagcccgggacattttgacacccccataatatttttccagaattaacagtataaattgcatctcttgttcaagagttccctatcactctctttaatcactactcacagtaacctcaactcctgcattccatgggttggatccgcgaaggcgcgcccat

SEQ ID NO: 26 - DNA, 604 bp, synthetic construct
  source 1..604: mol_type = other DNA
atcgatagatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcct

SEQ ID NO: 27 - DNA, 258 bp, synthetic construct
  source 1..258: mol_type = other DNA
taagtatgatcagcctcgagctagctggaaagattggaaagactggaaagataggaaacactggaaagagccattagatctcgagtggaagcttagacacgagcccgggacattttgacacccccataatatttttccagaattaacagtataaattgcatctcttgttcaagagttccctatcactctctttaatcactactcacagtaacctcaactcctgcattccatgggttggatccgcgaaggcgcgcccat

SEQ ID NO: 28 - DNA, 331 bp, synthetic construct
  source 1..331: mol_type = other DNA
taagtatgagctagcctggggctcgacagcactagtgaggaatttccattgaggaatttccattgaggaatttccattagatcgttgaggaatttccattgaggaatttccattgaggaatttccattagatcgttgaggaatttccattgaggaatttccattgaggaatttccattagatcgttggggactttccactggggactttccactggggactttccactggggactttccactggggactttccactagatctagactcgagtggaagcttagacactagagggtatataatggaagctcgacttccagaacatatgccacc

SEQ ID NO: 29 - DNA, 295 bp, synthetic construct
  source 1..295: mol_type = other DNA
tggaaagattggaaagactggaaagatccattagatcgtttggaaagagaggaaacactggaaagagtcagcctcataggtggaaagataggaaacactggaaagagccattagatctcgagtggaagcttagacacgagcccgggacattttgacacccccataatatttttccagaattaacagtataaattgcatctcttgttcaagagttccctatcactctctttaatcactactcacagtaacctcaactcctgcattccatgggttggatccgcgaaggcgcgcccat

SEQ ID NO: 30 - DNA, 233 bp, synthetic construct
  source 1..233: mol_type = other DNA
tggaaagattggaaagactggaaagataggaaacactggaaagagccattagatctcgagtggaagcttagacacgagcccgggacattttgacacccccataatatttttccagaattaacagtataaattgcatctcttgttcaagagttccctatcactctctttaatcactactcacagtaacctcaactcctgcattccatgggttggatccgcgaaggcgcgcccat

SEQ ID NO: 31 - DNA, 295 bp, synthetic construct
  source 1..295: mol_type = other DNA
gaggaatttccattgaggaatttccattgaggaatttccattagatcgttgaggaatttccattgaggaatttccattgaggaatttccattagatcgttgaggaatttccattgaggaatttccattgaggaatttccattagatcgttggggactttccactggggactttccactggggactttccactggggactttccactggggactttccactagatctagactcgagtggaagcttagacactagagggtatataatggaagctcgacttccagaaggccgcgacat

SEQ ID NO: 32 - DNA, 607 bp, synthetic construct
  source 1..607: mol_type = other DNA
taaatcgatagatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcct

SEQ ID NO: 33 - DNA, 398 bp, synthetic construct
  source 1..398: mol_type = other DNA
tggaaagactggaaagatccattagatcgtttggaaagagaggaaacactggaaagagtcagcctcataggtggaaagataggaaacactggaaagagccattagatctcgagtggaagcttagacacgagcccgggacattttgacacccccataatatttttccagaattaacagtataaattgcatctcttgttcaagagttccctatcactctctttaatcactactcacagtaacctcaactcctgcattccatgggttggatccgcgaaggcgcgcccatatggcccagtccaagcacggcctgaccaaggagatgaccatgaagtaccgcatggagggctgcgtggacggccacaagttcgtgatcaccggcgagggcatcggctacccct

SEQ ID NO: 34 - protein, 37 aa, synthetic construct
MAQSKHGLTKEMTMKYRMEGCVDGHKFVITGEGIGYP

SEQ ID NO: 35 - DNA, 342 bp, synthetic construct
  source 1..342: mol_type = other DNA
tggaaagattggaaagactggaaagataggaaacactggaaagagccattagatctcgagtggaagcttagacacgagcccgggacattttgacacccccataatatttttccagaattaacagtataaattgcatctcttgttcaagagttccctatcactctctttaatcactactcacagtaacctcaactcctgcattccatgggttggatccgcgaaggcgcgcccatatggcccagtccaagcacggcctgaccaaggagatgaccatgaagtaccgcatggagggctgcgtggacggccacaagttcgtgatcaccggcgagggcatcggctacc

SEQ ID NO: 36 - protein, 36 aa, synthetic construct
MAQSKHGLTKEMTMKYRMEGCVDGHKFVITGEGIGY

SEQ ID NO: 37 - DNA, 356 bp, synthetic construct
  source 1..356: mol_type = other DNA
gaggaatttccattgaggaatttccattgaggaatttccattagatcgttgaggaatttccattgaggaatttccattgaggaatttccattagatcgttgaggaatttccattgaggaatttccattgaggaatttccattagatcgttggggactttccactggggactttccactggggactttccactggggactttccactggggactttccactagatctagactcgagtggaagcttagacactagagggtatataatggaagctcgacttccagaaggccgcgacatatggcccagtccaagcacggcctgaccaaggagatgaccatgaagtaccgcatggagggct

SEQ ID NO: 38 - protein, 20 aa, synthetic construct
MAQSKHGLTKEMTMKYRMEG

SEQ ID NO: 39 - DNA, 89 bp, synthetic construct
  source 1..89: mol_type = other DNA
ggactttccactggggactttccactggggactttccactggggactttccactagatctagactcgagtggaagcttagacactagag

SEQ ID NO: 40 - DNA, 254 bp, synthetic construct
  source 1..254: mol_type = other DNA
agtatgagctagcctggggctcgacagcactagtgaggaatttccattgaggaatttccattgaggaatttccattagatcgttgaggaatttccattgaggaatttccattgaggaatttccattagatcgttgaggaatttccattgaggaatttccattgaggaatttccattagatcgttgggacactagagggtatataatggaagctcgacttccagaaggccgcgacatatgccaccatggcccagt